(19) United States
(12) Patent Application Publication
(10) Pub. No.: US 2026/0265752 A1
(43) Pub. Date: Sep. 10, 2026

(54) COMPOSITIONS AND METHODS USEFUL FOR THE TREATMENT AND MANAGEMENT OF OBESITY ASSOCIATED METABOLIC DYSFUNCTION

(71) Applicant: THE CHILDREN'S HOSPITAL OF PHILADELPHIA, Philadelphia, PA (US)

(72) Inventor: David Hill, Philadelphia, PA (US)

(73) Assignee: THE CHILDREN'S HOSPITAL OF PHILADELPHIA, Philadelphia, PA (US)

(21) Appl. No.: 19/165,866

(22) PCT Filed: Mar. 18, 2024
(86) PCT No.: PCT/US2024/020417

§ 371 Date: Sep. 16, 2025

Related U.S. Application Data
(60) Provisional application No. 63/490,880, filed on Mar. 17, 2023.

(51) Int. Cl. C12N 15/113; A61P 3/10; C12Q 1/6883

(52) U.S. Cl. CPC C12N 15/1135; A61P 3/10; C12Q 1/6883; C12N 2310/141; C12N 2320/32; C12Q 2600/118; C12Q 2600/158; C12Q 2600/178

(57) ABSTRACT

Compositions and methods for the management and treatment of obesity related metabolic dysfunction disorders are disclosed.

### CROSS-REFERENCE TO RELATED APPLICATIONS

This application claims priority of U.S. Provisional application No. 63/490,880 filed on Mar. 17, 2023, the entire contents being incorporated herein by reference as though set forth in full.

### Government Support Statement

This invention was made with government support under grant numbers K08DK116668, R03 DK129418, and R01 DK49780 awarded by the National Institutes of Health. The government has certain rights in the invention.

### Incorporation-by-Reference of Material Submitted in Electronic Form

The Contents of the electronic sequence listing (CHOP-145-PCT.xml; Size: 79,507 bytes; and Date of Creation: Mar. 18, 2024) is herein incorporated by reference in its entirety.

### FIELD OF THE INVENTION

The present invention relates the fields of miRNA regulation and obesity associated metabolic dysfunction. More specifically, the invention provides therapeutics comprising variant miR-6236 molecules and methods of use thereof for the treatment of cardiovascular disease, diabetes, and other obesity related disorders.

### BACKGROUND OF THE INVENTION

Several publications and patent documents are cited throughout the specification in order to describe the state of the art to which this invention pertains. Each of these citations is incorporated by reference herein as though set forth in full.

Adipose tissue is evolved to store energy during periods of excess caloric intake and release energy during periods of caloric deprivation. This process is tightly regulated by insulin, a hormone that promotes lipogenesis and inhibits lipolysis with the ultimate function of maintaining global energy homeostasis (Santoro et al., 2021). However, periods of excessive and extended caloric intake can lead to obesity, tissue insulin resistance, and Type 2 Diabetes (T2D) (Blüher, 2013). Though the mechanisms of this pathologic cascade are not completely understood, adipose tissue insulin resistance may be a contributing feature to T2D (Abel et al., 2001). As a result, molecules with potential to restore adipose tissue insulin sensitivity are of high interest as potential T2D therapeutics (Kusminski et al., 2016).

Adipose tissue undergoes dramatic remodeling during obesity including adipocyte hypertrophy, vascularization, and immune cell accumulation (Sung et al., 2013). The past decade has witnessed a revolution in our understanding of how immune cells influence adipose tissue remodeling and function in the context of obesity (Mathis, 2013). For example, adipose tissue macrophages (ATMs) are now understood to be a heterogeneous immune cell population which can have harmful or beneficial effects on adipose tissue functions depending on context (Russo and Lumeng, 2018). We recently identified CD9+lipid-associated macrophages (LAMs), a metabolically activated ATM population that accumulates in obese adipose tissue of mice and humans (Hill et al., 2018; Jaitin et al., 2019). This ATM subset has predominantly beneficial functions in the context of obesity by promoting adipose tissue functions and global metabolic homeostasis (Jaitin et al., 2019). However, the mechanisms by which LAMs exert these important functions in the context of obesity are not well understood.

As noted above, LAMs express CD9 (Hill et al., 2018), a tetraspanin involved in extracellular vesicle (EV) biogenesis, packaging, release, and uptake on the cell surface (Akers et al., 2013). We have also found that LAMs secrete large quantities of EVs (Hill et al., 2018)—a cellular feature central to ATM functions in other contexts (Ying et al., 2017, 2021). One mechanism by which EV release may mediate ATM functions is through the secretion and delivery of microRNAs (miRNAs) to other cell types. miRNAs are small, noncoding RNA molecules that are transcribed as primary miRNAs (pri-miRNAs), and subsequently cleaved to generate precursor miRNAs (pre-miRNAs) and mature miRNAs (O'Brien et al., 2018). miRNAs regulate a target gene's translation by binding to the 3′ untranslated region (UTR) of its mRNA product (Eulalio et al., 2008) which leads to its suppression or degradation.

Diabetes-related cardiovascular disease (CVD) is a global health issue that causes thousands of people's death around the world annually. Diabetes-related CVD is still prevailing despite the progression being made in its diagnosis and treatment. Therefore, it is urgent to find therapeutic strategies to prevent or treat it.

### SUMMARY

In accordance with the present invention, a composition comprising a synthetic hsa-miR6236 or functional variant thereof in a pharmaceutically acceptable carrier for is provided for treatment of one or more obesity related metabolic disorders in a human subject in need of treatment thereof. Disorders to be treated include without limitation: obesity, metabolic syndrome, type 2 diabetes, non-alcoholic fatty liver disease, hyperinsulinemia, hypoinsulinemia, hypertension, hyperhepatosteatosis, hyperuricemia, fatty liver, polycystic ovarian syndrome, hyperphagia, acanthosis nigricans, endocrine abnormalities, triglyceride storage disease, Bardet-Biedl syndrome, Lawrence-Moon syndrome, Parder-Labhart-Willi syndrome, a primary mitochondrial genetic disorder, a neurological disease, and an age-associated pathology.

The composition can be used to advantage for modulation of PTEN activity in a tissue selected from brown fat, white fat, subcutaneous adipose tissue, liver and muscle. In certain approaches, the synthetic hsa-miR6236 comprises chemical modifications for enhancing stability and, or, bioavailability in the body, and alleviates symptoms of said one or more obesity related metabolic disorders. The synthetic miR6236 or functional variant thereof can be operably linked to a nanoparticle or encapsulated within an exosome. The synthetic miRNA can operably linked to targeting molecule for specific delivery to a specific cell type or tissue. In certain embodiments, the synthetic miRNA6236 or functional variant thereof binds PTEN mRNA thereby inhibiting PTEN protein expression and causing weight loss.

Also disclosed is an exosome pellet or physiological solution isolated from a biological sample comprising exosomes harboring miRNA6236 or a functional variant thereof wherein the pellet or solution is essentially free from undesirable entities having a diameter less than 20 nm and greater than 140 nm. In a preferred embodiment, the exosome containing pellet or solution is obtained from CD9+LAM cells. Alternatively, the pellet or solution or free miRNA can be obtained from a biological sample, preferably, a blood sample. In certain aspects, the pellet or solution can further comprise an exogenous therapeutic metabolic product

In yet another aspect, a method of treating obesity related metabolic dysfunction in a subject in need thereof is provided comprising administering an effective amount of the composition or exosome pellet or solution described above to a subject in need thereof. The composition can be administered via a route selected from systemic, intramuscular, topical, oral, parenteral, transdermal patch, aerosolized, pulmonary, ophthalmic, buccal, and lingual administration.

Additionally, diagnostic and, or prognostic methods are disclosed for identifying subjects at altered risk for one or more obesity related metabolic disorders. In one approach, the method comprises detecting levels of free hsa-miR6236 or endosomal encapsulated hsa-miR6236 in a biological sample obtained from a subject; and comparing the levels detected with those from control subjects without disease and from subjects with at least one obesity related metabolic disorder, thereby identifying a subject as being at greater or lesser risk for an obesity related metabolic disorder. In certain aspects, when reduced levels of miR6236 levels are detected in the subject relative to a control subject without disease the subject is identified as having an increased risk of the metabolic disorder. Conversely, when elevated levels of miR6236 levels are detected in the subject relative to a control subject without disease, the subject is identified as having a decreased risk of having or developing a metabolic disorder.

### BRIEF DESCRIPTION OF THE DRAWINGS

FIGS. 1A-10. miR-6236 is a LAM-secreted miRNA that is enriched in adipose tissue and regulated by obesity. (FIG. 1A) Flow-cytometric identification of CD9+LAMs in epididymal white adipose tissue (eWAT) of obese mice. (FIG. 1B) The 20 most abundant miRNAs in the EV fraction of sort purified CD9+LAMs as detected by small RNA sequencing (RNA-seq). (FIG. 1C) Genomic alignment of reads from small RNA-Seq and Argonaute HITS-CLIP-Seq. MiR-6236 locus with historically annotated pre-miR-6236 and mature miR-6236 regions indicated via the blue and grey bars, respectively. (FIG. 1D). Pre-miR-6236 secondary stem-loop structure. (FIG. 1E) Expression level of miR-6236 in mice tissues measured by small RNA-seq. Abbreviations: RPM: Read per Million; mWAT: mesenteric white adipose tissue; BAT: brown adipose tissue; aWAT: intra-abdominal white adipose tissue; iWAT: inguinal white adipose tissue, eWAT: epididymal white adipose tissue, BM: bone marrow. (FIG. 1F) Expression level of miR-6236 in EV fractions isolated from adipose tissue macrophages (ATMs) of lean (CFD) or obese (HFD) mice (n=3-4). Data representative of >2 experiments for panels in FIGS. 1A, 1B, and 1E. Data in panels in FIGS. 1E and F presented as ‘mean±SEM’. (FIG. 1G) Nanoparticle Tracking Analysis (NTA) of macrophage-derived extra-cellular vesicles (EVs) isolated via purification kit or ultracentrifugation. (FIG. 1H) miR-6236 expression in sort-purified myeloid immune cells from eWAT of DIO male mice (n=3-4). (FIG. 1I) miR-6236 expression in eWAT macro-phage and monocyte (mono)-derived EVs of DIO male mice. (FIG. 1J) NTA of eWAT macrophage-derived EVs (n=4). (FIG. 1K) Non-reducing CD9 Western blot in EV and cellular fractions of LAMs from DIO male mice (n=3). (FIG. 1L) miR-6236 expression in DIO, Trem2 sufficient (WT) or deficient (KO) Kupffer cells (n=4). (FIG. 1M) miR-6236 expression in serum of young (6 month) and aged (24 month) mice (n=4-5). (FIG. 1N) miR-6236 expression in BMDMs under different activation conditions (n=3). (FIG. 10) Legacy PPARγ ChIP-seq browser-tracks in macrophages and ATAC-seq tracks in CD9+LAMs at the miR-6236 gene locus. Data present-ed as mean±SEM where appropriate; ns=not significant, *p<0.05, **** p <0.0001, Student's t test.

FIGS. 2A-2Y. Development and metabolic phenotype of miR-6236 KO mice and whole body deletion of miR-6236 exacerbates obesity-associated metabolic outcomes. (FIG. 2A)

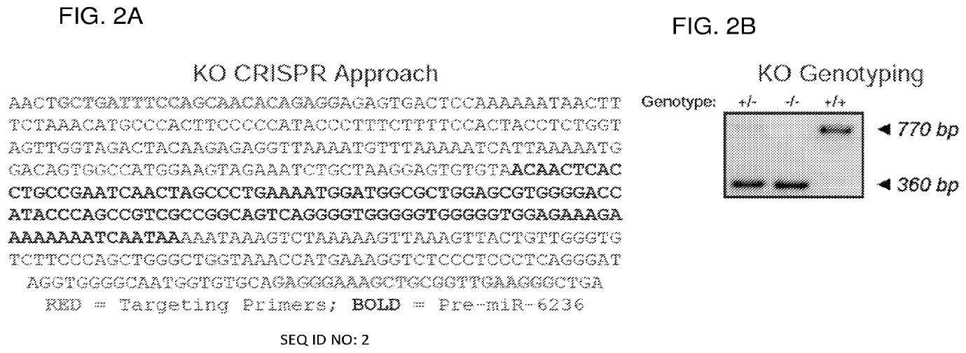

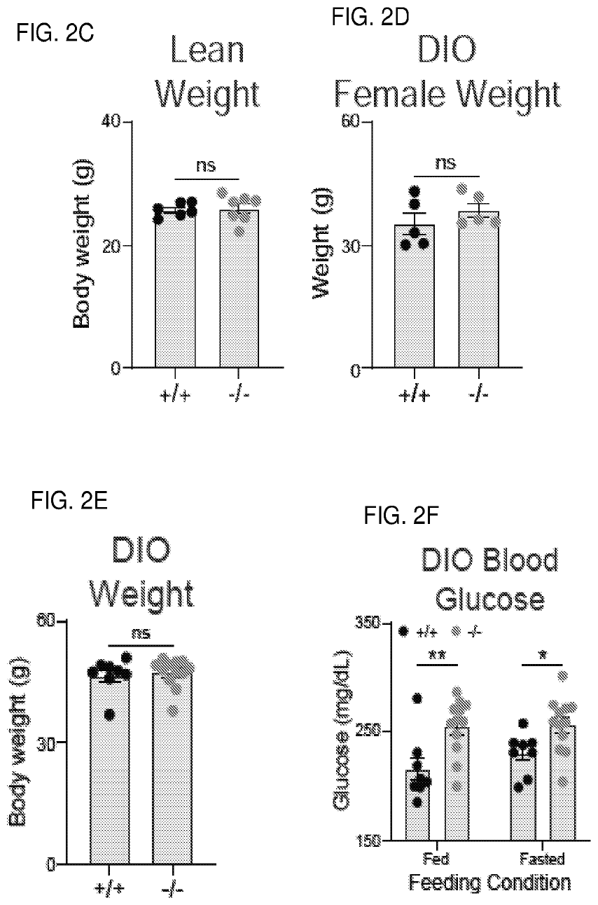

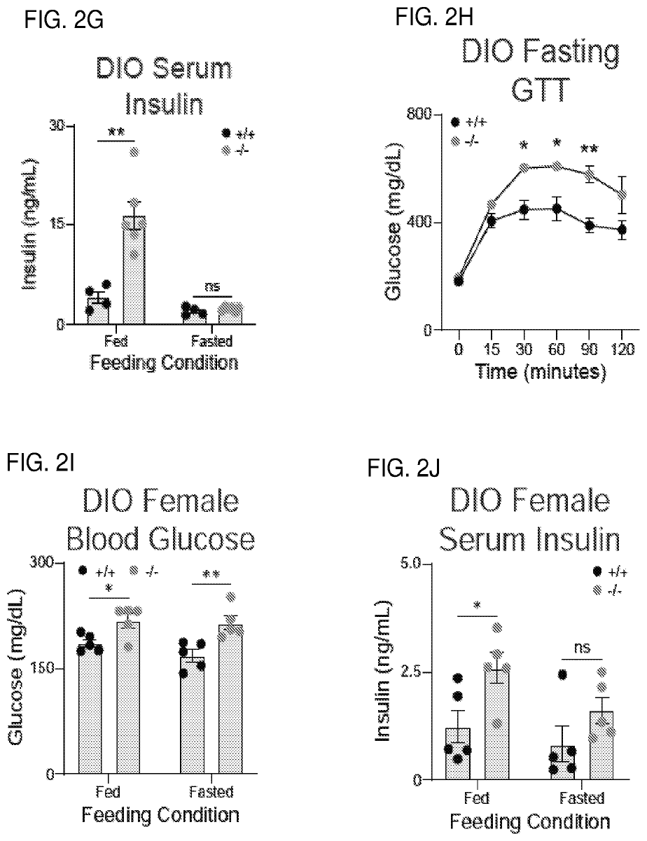

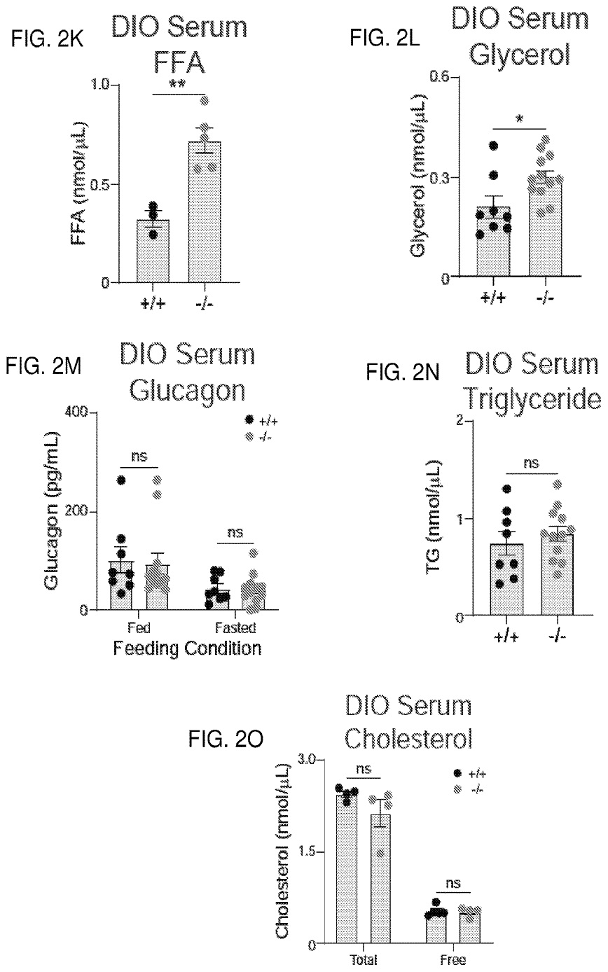

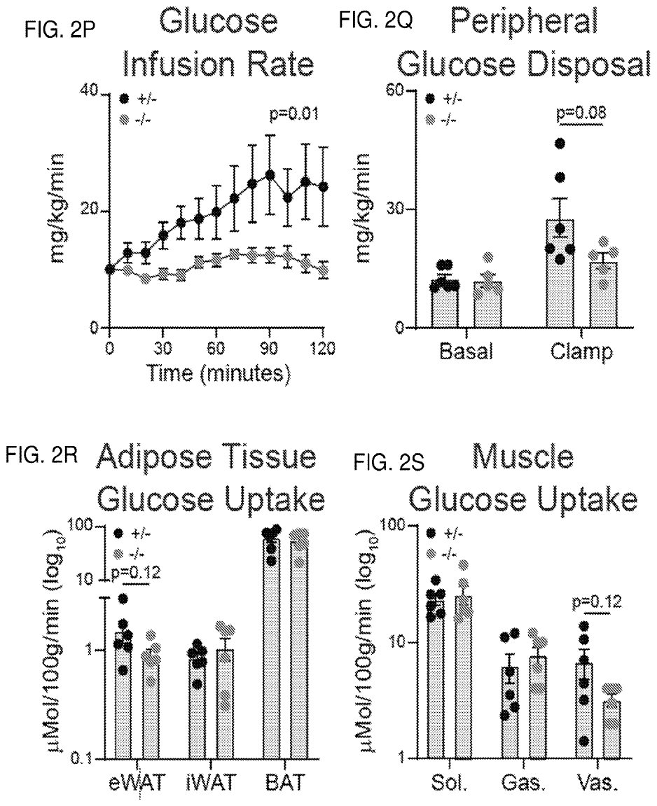

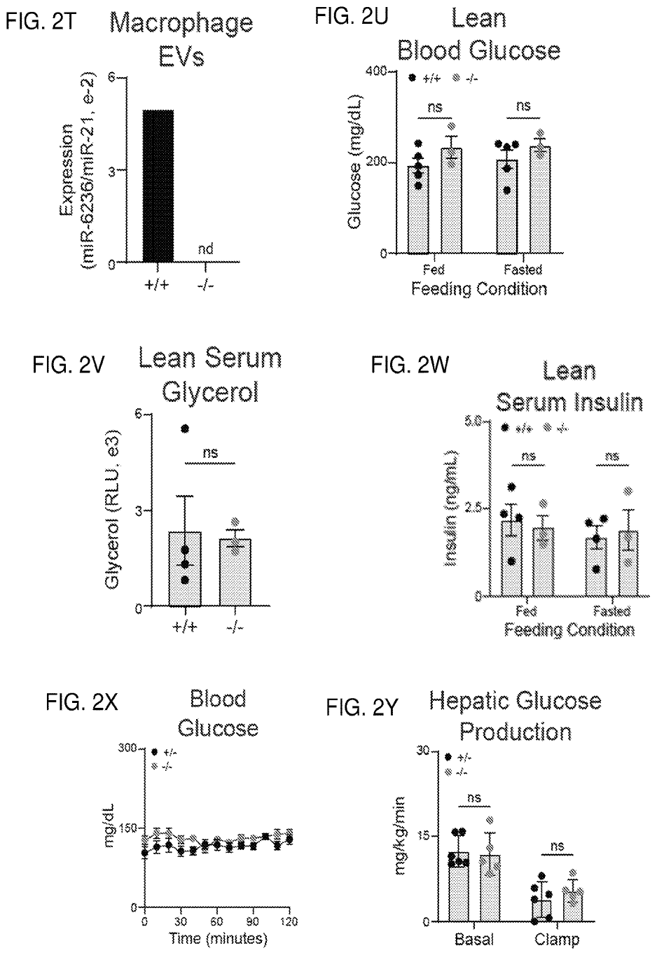

Genomic locus of miR-6236 showing pre-miR-6236 sequence (bold) and flanking sequences. Region between CRISPR targeting primers is deleted in whole body knockout mice. (FIG. 2B) Genotyping gel image showing expected band size of the miR-6263 DNA locus in heterozygous (+/−), knockout (−/−), and wild type (+/+) mice. (FIG. 2C) Body weight of lean male mice fed normal chow diet for 18 weeks (n=6-7). (FIG. 2D) Body weight of DIO female mice fed high fat diet for 12 weeks (n=5). (FIG. 2E) Body weight wild type (+/+) or miR-6236 knockout (−/−) male mice subjected to high fat diet-induced obesity (DIO) (n=8-12). (FIG. 2F) Fed and fasted (5 hrs.) blood glucose level in DIO male mice (n=8-12). (FIG. 2G) Fed and fasted (5 hrs.) serum insulin level in DIO male mice (n=4). (FIG. 2H) Intraperitoneal glucose tolerance test in DIO male mice (n=5). (FIG. 21) Fed and fasted (5 hrs.) blood glucose level in DIO female mice (n=5). (FIG. 2J) Fed and fasted (5 hrs.) serum insulin level in DIO female mice (n=5). (FIG. 2K) Serum free fatty acid (FFA) level in DIO male mice (n=4). (FIG. 2L) Serum glycerol level in DIO male mice (n=8-12). (FIG. 2M) Serum glucagon level in DIO male mice (n=8-12). (FIG. 2N) Serum triglyceride level in DIO male mice (n=8-12). (FIG. 20) Serum cholesterol level in DIO male mice (n=4). (FIG. 2P-2S) Glucose infusion rate (FIG. 2P), peripheral glucose disposal (FIG. 2Q), adipose tissue glucose uptake (FIG. 2R), and muscle glucose uptake (FIG. 2S) as determined during a fasting hyperinsulinemic-euglycemic clamp in DIO male mice of +/−or −/− genotype (n=5-6). (FIG. 2T) miR-6236 expression in EVs of BMDMs. (FIG. 2U), Fed and fasted (5 hrs.) blood glucose level and (FIG. 2V) serum glycerol level in lean male mice. (FIG. 2W) Serum triglyceride level in DIO male mice (n=4-12). FIGS. 2X-2Y, Blood glucose (FIG. 2X) and hepatic glucose production (FIG. 2Y) as determined during a hyperinsulinemic-euglycemic clamp in fasted DIO male mice (n=5-6). Sol. =soleus, Gas. =gastrocnemius, Vas. =vastus Serum cholesterol level in DIO male mice (n=4). Data representative of ≥2 experiments with exception of clamp which is 1 experiment. Data presented as mean±SEM; ns =not significant, *p<0.05, ** p<0.01, Student's t test and mixed effects model with interaction term (GIR)

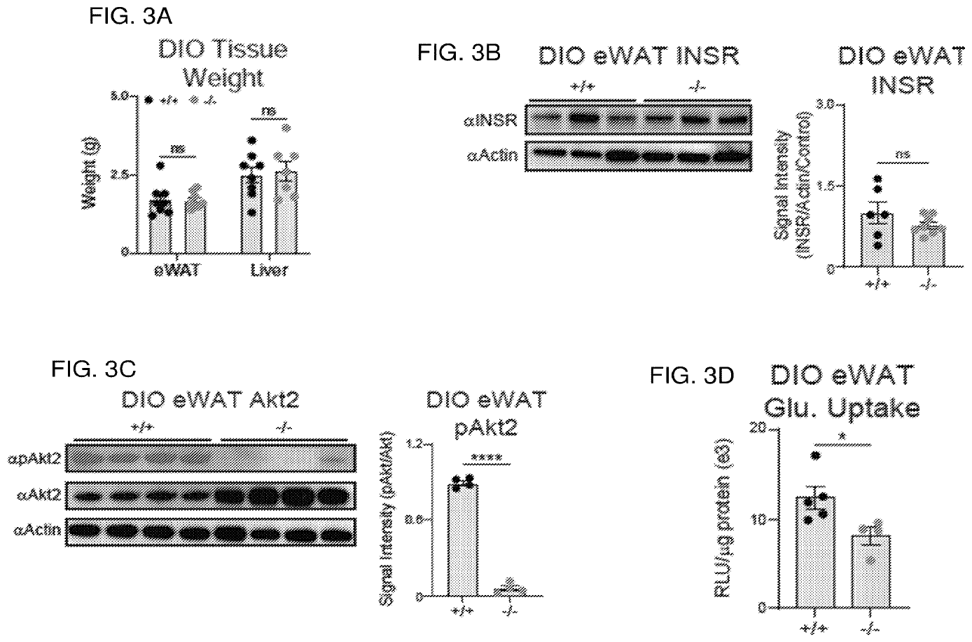

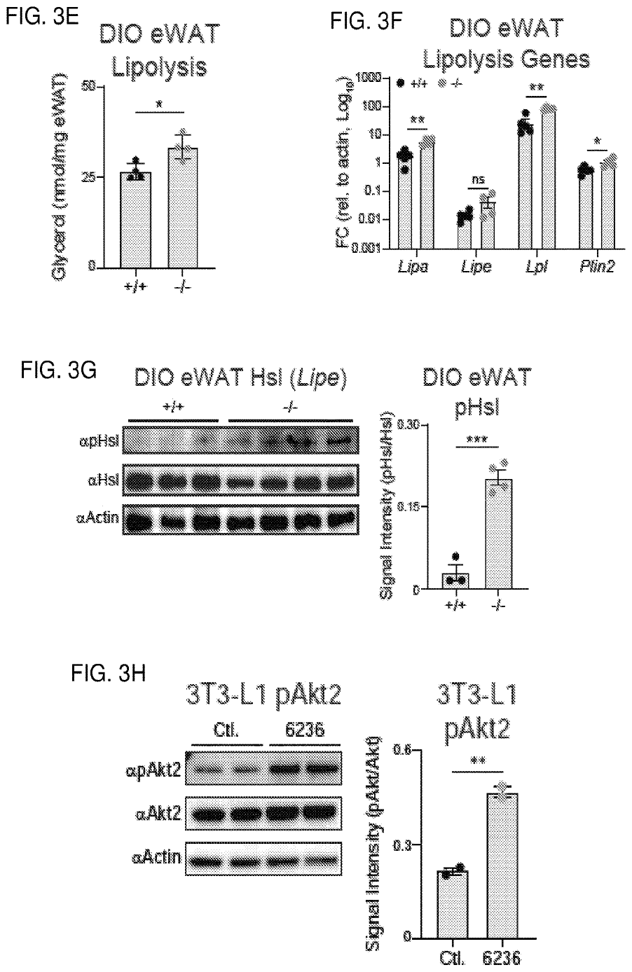

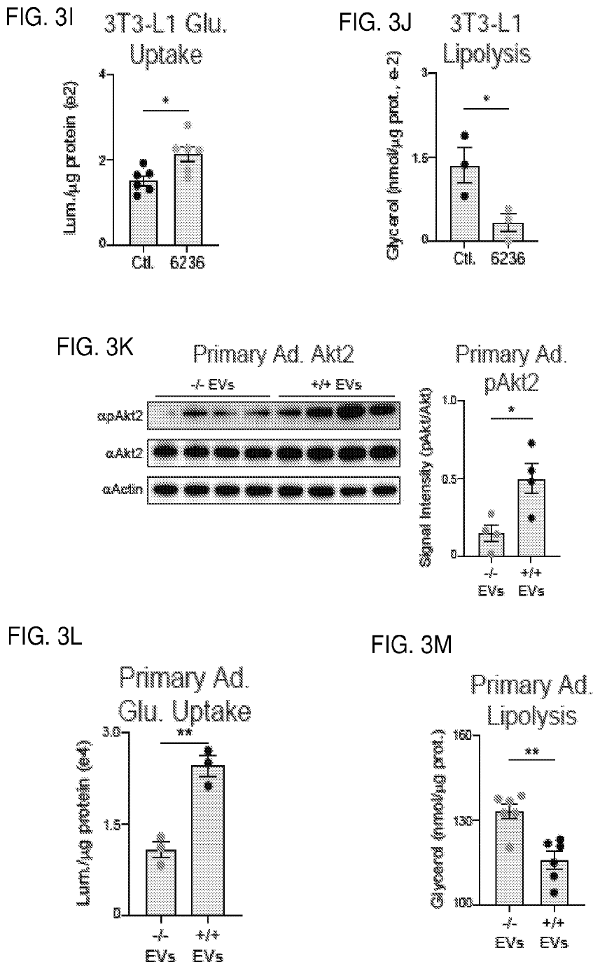

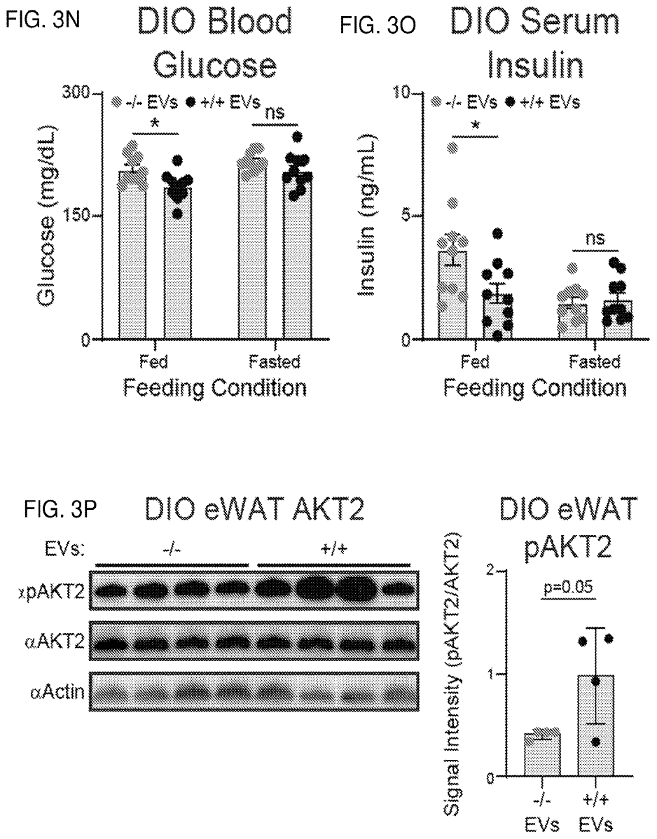

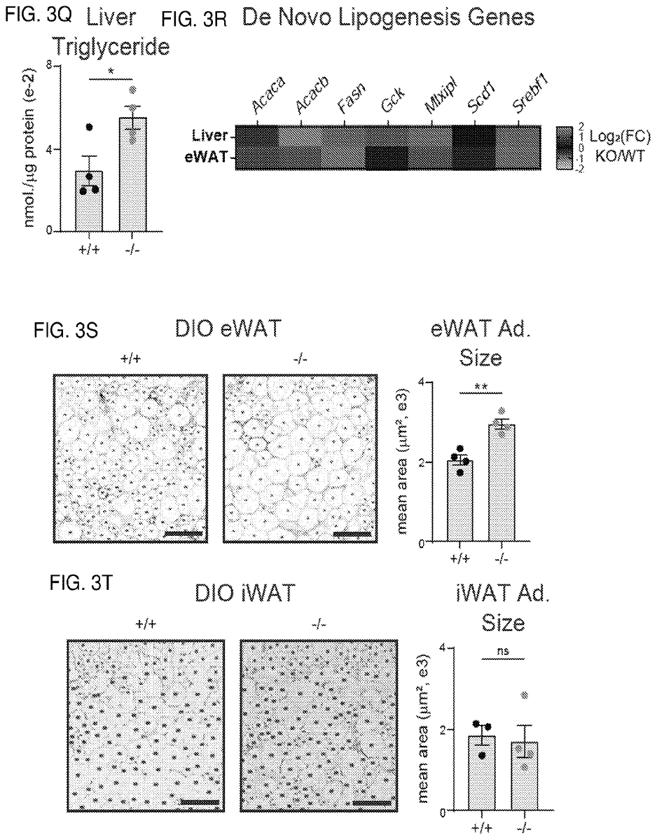

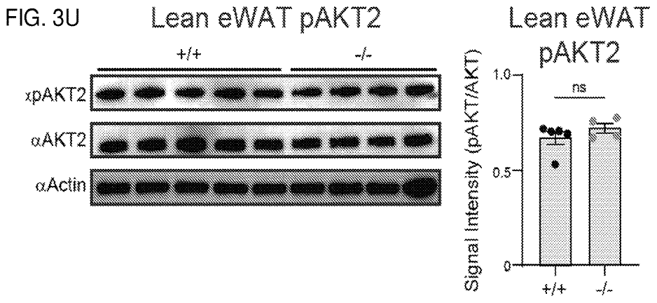

FIGS. 3A-3U. Adipose tissue features in miR-6236 KO mice and miR-6236 promotion insulin-mediated signaling and functions in adipocytes. (FIG. 3A) eWAT and liver weight of DIO wild type (+/+) or miR-6236 knockout (−/−) male mice (n=7-8). (|FIG. 3B) Insulin receptor (InsR) Western blot and quantification in eWAT of DIO male mice (n=3). (FIG. 3C) Akt2 and pAkt2 Western blot and quantification in eWAT of DIO WT (+/+) or KO (−/−) male mice (n=4). (FIG. 3D) Insulin-stimulated glucose uptake in mature adipocytes isolated from eWAT of DIO WT or KO male mice (n=2-3). (FIG. 3E) Glycerol release from eWAT adipocytes of DIO WT or KO male mice in the presence of insulin (n=4). (FIG. 3F) Expression of key lipolysis genes in eWAT of DIO WT or KO male mice (n=4-5). (FIG. 3G) Hsl and pHsl Western blot and quantification in eWAT of DIO WT or KO male mice (n=3-4). (FIG. 3H) Akt2 and pAkt2 Western blot and quantification in 3T3-L1 adipocytes transfected with miR-6236 mimic or scrambled control (Ctl.) (n=2). (FIG. 31) Insulin-stimulated glucose uptake in 3T3-L1 adipocytes transfected with miR-6236 mimic or control (n=6). (FIG. 3J) Glycerol release from 3T3-L1 adipocytes transfected with miR-6236 mimic or control in the presence of insulin (n=3). (FIG. 3K) Akt2 and pAkt2 Western blot and quantification in WT primary adipocytes treated with EVs isolated from KO or WT BMDMs (n=4). (FIG. 3L) Insulin-stimulated glucose uptake in WT primary adipocytes treated with EVs isolated from KO or WT BMDMs (n=3). (FIG. 3M) Glycerol release from WT primary adipocytes treated with EVs isolated from KO or WT BMDMs (n=6). (FIGS. 3N-3P) Fed and fasted (5 hours) blood glucose (FIG. 3N) and serum insulin (FIG. 30) levels, as well as fed AKT2 and pAKT2 Western blot and quantification (FIG. 3P), in DIO WT male mice injected i.p. with EVs from KO or WT BMDMs. (FIG. 3Q) Triglyceride content of liver from DIO male mice (n=4). (FIG. 3R) Heatmap of de novo lipogenesis-related gene expression in liver and eWAT of DIO wild type or miR-6236 knockout male mice fed ad libitum (n=4). H&E sections and adipocyte size calculations from eWAT (FIG. 3S) or iWAT (FIG. 3T) of DIO male mice (n=3-4, bar=200 μm). (FIG. 3U) AKT2 and pAKT2 Western blot and quantification in eWAT of lean male mice (n=4-5). Data representative of >2 experiments. Data presented as ‘mean±SEM’; * p<0.05, ** p<0.01, *** p<0.001, Student's t test.

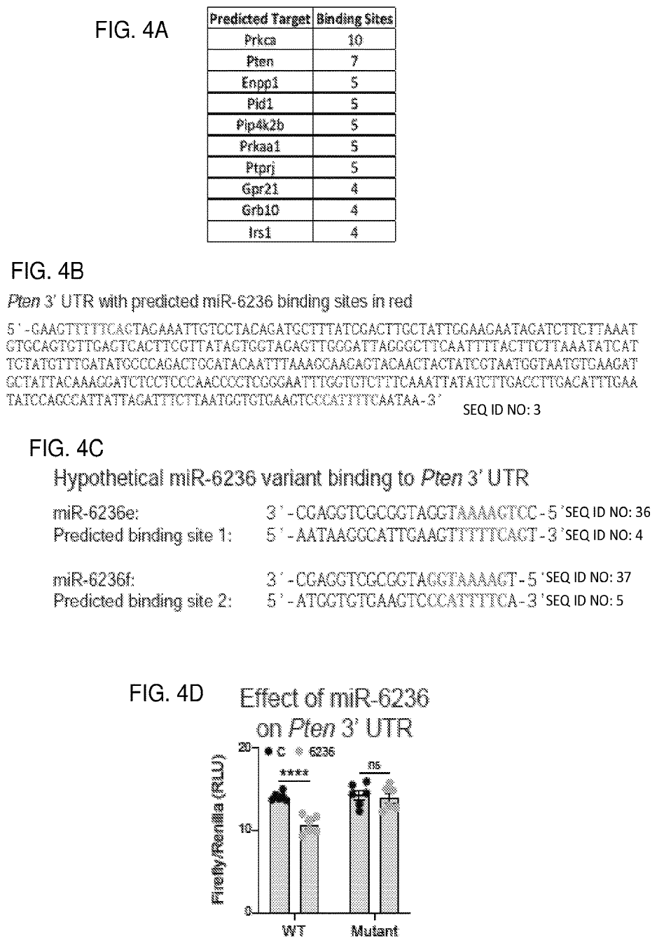

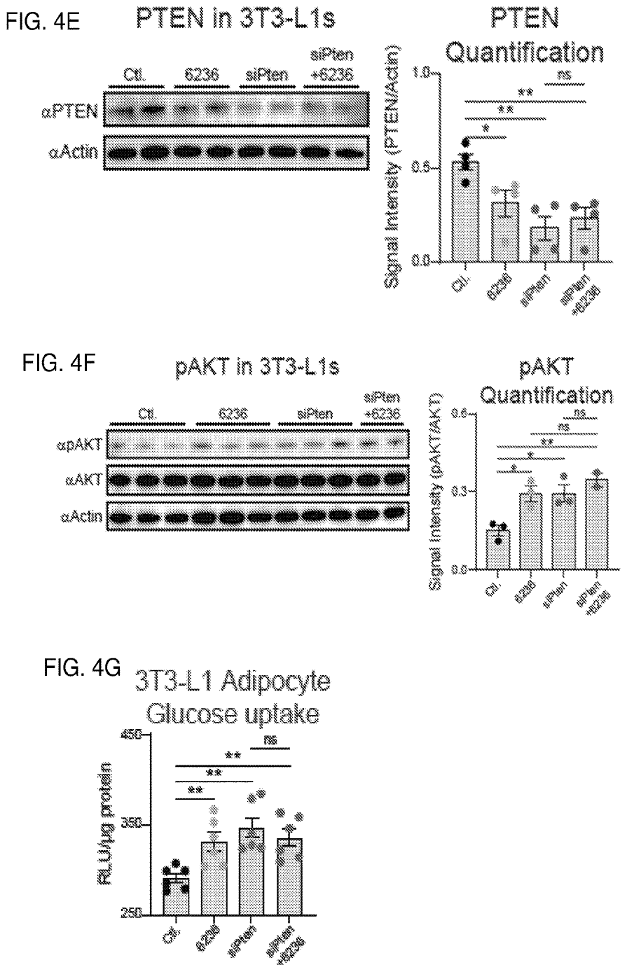

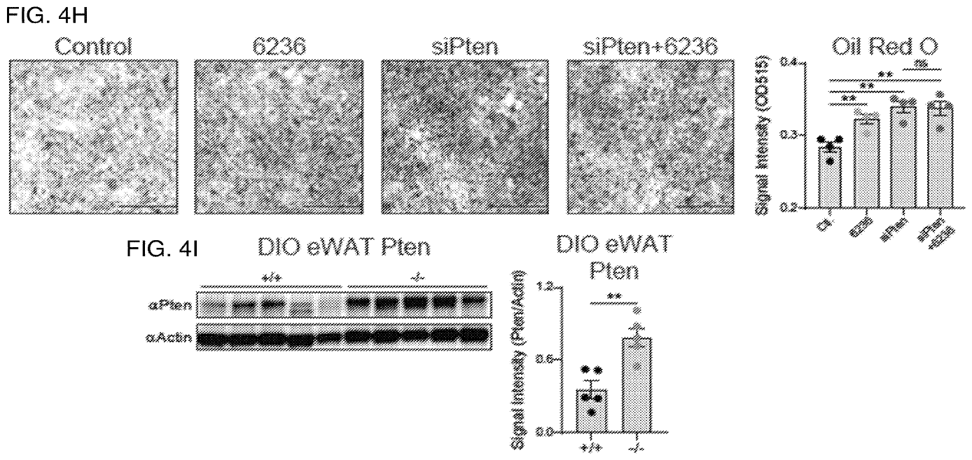

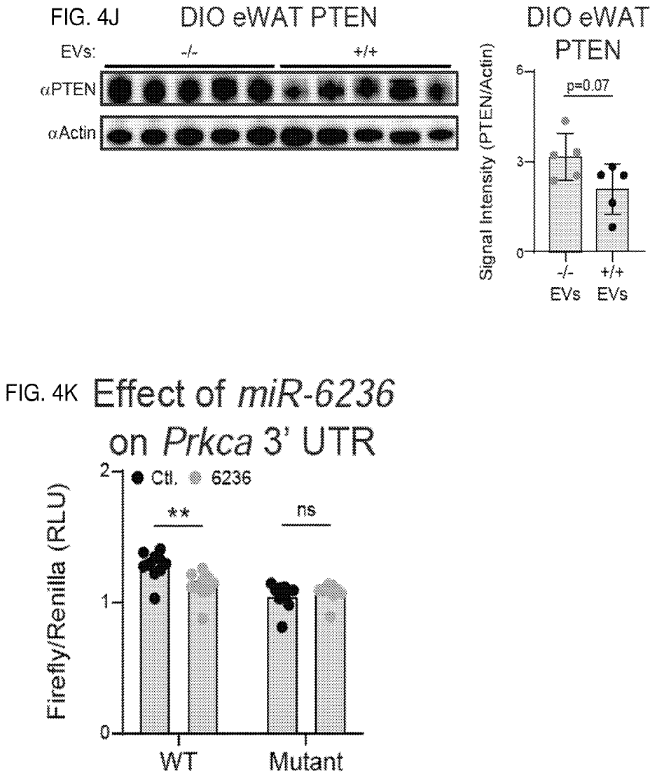

FIGS. 4A-4K. miR-6236 binding sites in PTEN UTR, and regulation of Akt2 phosphorylation by miR-6236 mimic and PTEN siRNA and correlation of miR-6236 promotion of adipocyte insulin signaling by inhibiting PTEN. (FIG. 4A) Predicted miR-6236 targets with binding site number. (FIG. 4B) Portion of 3′UTR cloned into pmirGLO for target validation. Red: predicted miR-6236 binding sites. (FIG. 4C) Hypothetical ‘miRNA-6236: PTEN 3′UTR’ binding. (FIG. 4D) Dual luciferase reporter assay to validate PTEN as miR-6236 target. Wild type (WT) or miR-6236 binding sites deleted (Mut) PTEN 3′UTR cloned in pmirGLO plasmid downstream of firefly ORF (n=6). (FIG. 4E) PTEN Western blot and quantification in 3T3-L1 cells. Abbreviations: Ctl: scrambled control; 6236: miR-6236 mimic; SiPten: PTEN siRNA (n=2). (FIG. 4F) Akt2 and pAkt2 Western blot and quantification in 3T3-11. Abbreviations: Ctl: scrambled control; 6236: miR-6236 mimic; SiPten: PTEN siRNA (n=2). (FIG. 4G) Insulin-stimulated glucose uptake in differentiated 3T3-L1 cells (n=6). (FIG. 4H) Oil Red O-stained 3T3-L1 cells and quantification (n=4). (FIG. 4I) PTEN Western blot and quantification in eWAT of DIO wild type (+/+) and knockout (−/−) male mice (n=5). (FIG. 4J) PTEN Western blot and quantification in wild type DIO male mice injected i.p. with EVs from knockout or wild type metabolically-activated BMDMs (n=5). (FIG. 4K) Dual luciferase reporter assay of scrambled control RNA (Ctl.) or miR-6236 (6236) binding to wild type (WT) or miR-6236 binding sites-deleted (Mutant) Prkca 3′UTR cloned in pmirGLO plasmid downstream of firefly ORF (n =10). Data representative of ≥2 experiments. Data presented as ‘mean±SEM’; *p<0.05, ** p<0.01, *** p<0.001, Student's t test.

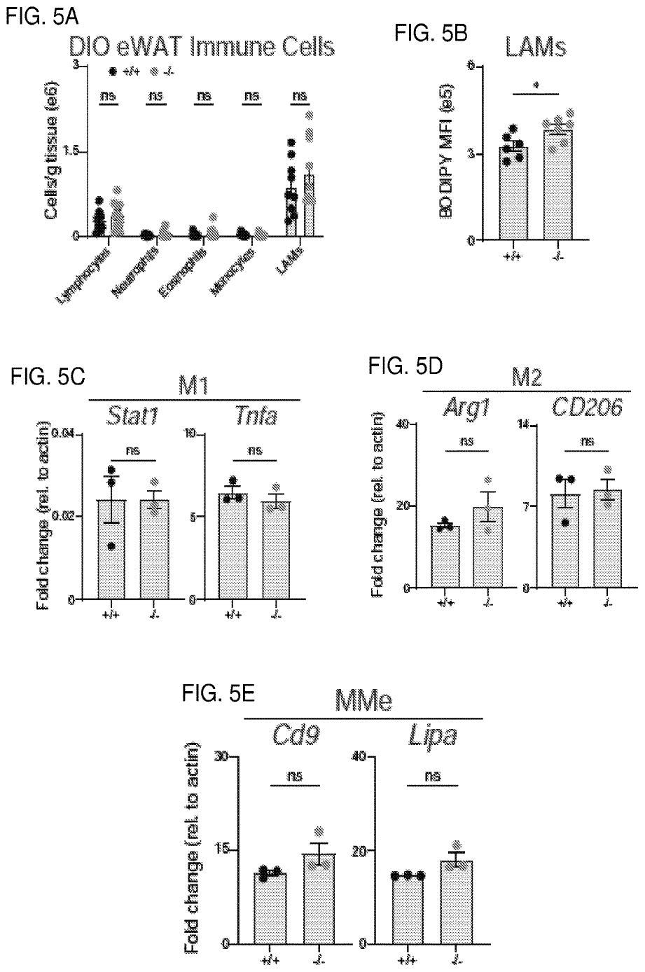

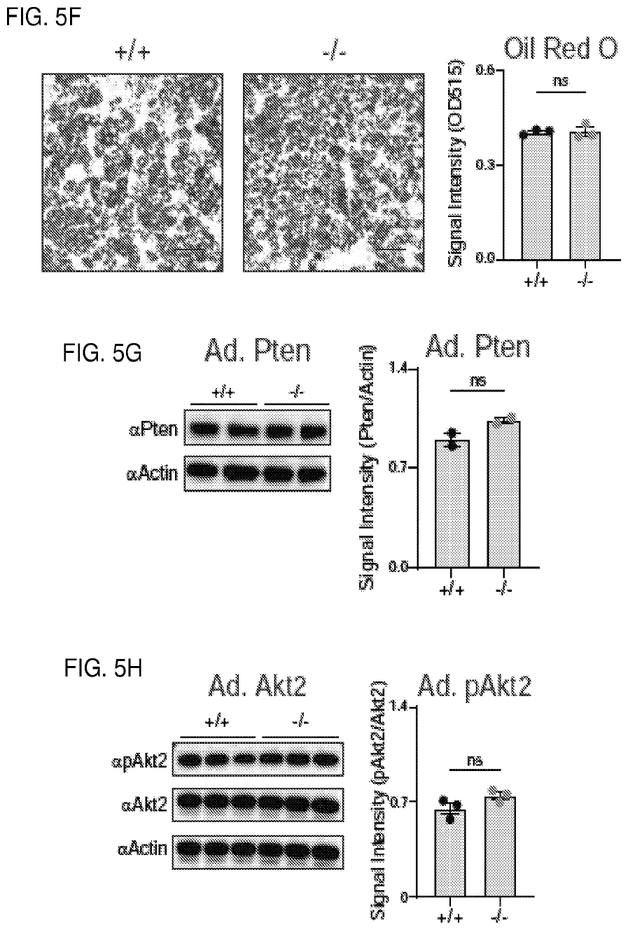

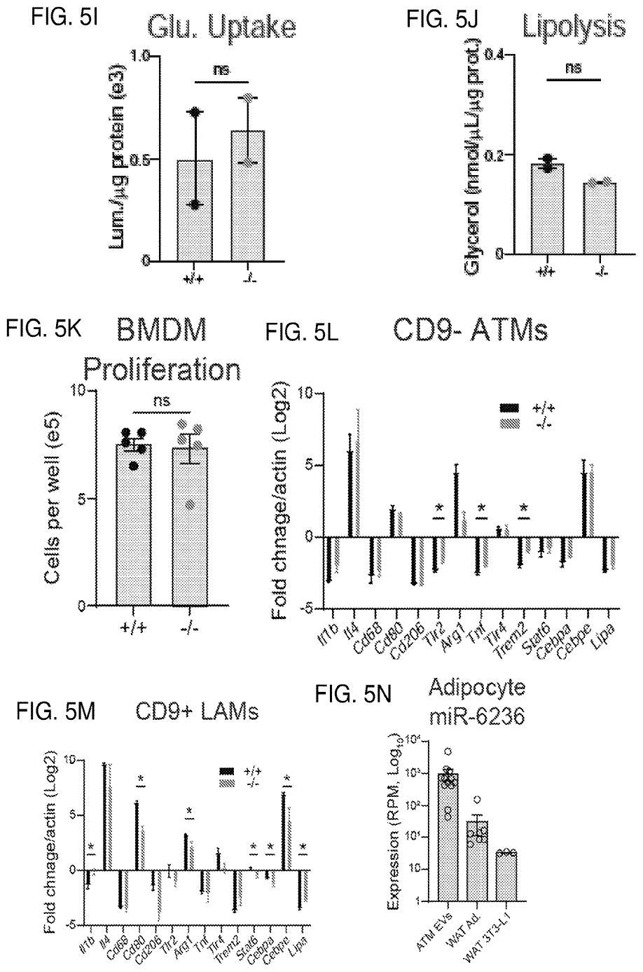

FIGS. 5A-5N. Macrophage and adipocyte-intrinsic effects of miR-6236. (FIG. 5A) Number of different immune cells determined by flow profiling in eWAT of DIO whole-body Knockout (−/−) and wild type littermate (+/+) male mice (n=6-7). (FIG. 5B) Total lipid content measured as BODIPY stain MFI (mean fluorescent intensity) in LAMs of DIO −/− and +/+male mice (n=6-7). (FIG. 5C-5E) Transcript abundance of key M1 (FIG. 5C), M2 (FIG. 5D) and metabolic activation (FIG. 5E) markers in ex vivo differentiated BMDM from −/− and +/+obese male mice. M1, M2 and metabolic activation was carried out ex vivo by LPS+IFN-γ, IL-4 and palmitate treatment respectively (n=3). (FIG. 5F) Oil Red O-stained primary adipocytes differentiated from primary preadipocytes of obese −/− and +/+male mice (left) and quantification of total Oil Red O stain (right) (n=3). (FIG. 5G) Western blot showing PTEN protein level in ex vivo differentiated primary adipocytes from DIO −/− and +/+male mice (left) and quantification of PTEN level in the blot (right) (n=2). (FIG. 5H) Western blot showing Akt2 and pAkt2 protein levels in ex vivo differentiated primary adipocytes from DIO −/− and +/+male mice (left), and quantification of pAkt2 level in the blot (right) (n=3). (FIG. 5I) Insulin-stimulated glucose uptake assay in ex vivo differentiated primary adipocytes from DIO −/− and +/+male mice (n=2). (FIG. 5J) Insulin-stimulated suppression of lipolysis in ex vivo differentiated primary adipocytes from DIO −/− and +/+male mice (n=2). (FIG. 5K) Cell counts per well of BMDM cultures (n=5). FIGS. 5L-5M, Expression of macrophage-related genes in CD9-ATMs (FIG. 5L) and CD9+LAMs (FIG. 5M) (n=3). FIG. 5N, Expression level of miR-6236 in ATM-derived EVs, white adipose tissue (WAT) adipocytes (Ad.), and 3T3-L1 adipocytes as measured by small RNA-seq (n=3-12). Data representative of >2 experiments. Data presented as ‘mean±SEM’; *p<0.05, ** p<0.01, *** p<0.001, Student's t test.

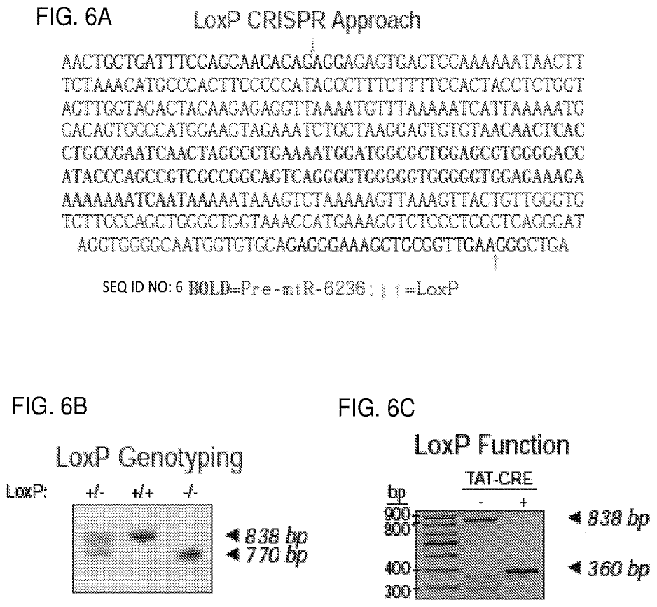

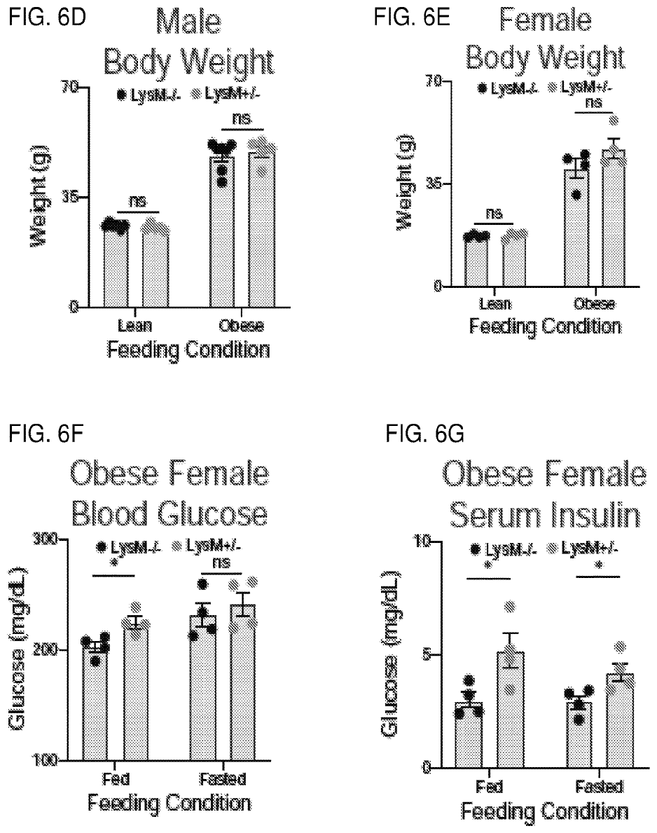

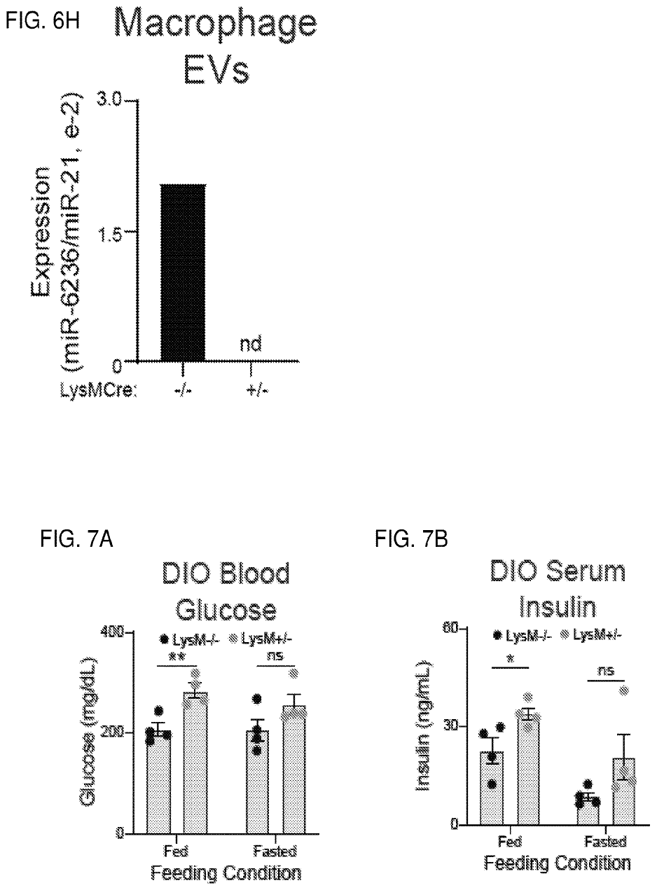

FIGS. 6A-6H. Generation and phenotyping of miR-6236//fl mice. (FIG. 6A) Genomic locus of miR-6236 showing pre-miR-6236 sequence (bold) and flanking sequences. Green arrows point to the location where loxP sequences were inserted to generate fl+/+ mice. (FIG. 6B) DNA PCR gel image showing expected band size of the miR-6263 DNA locus in fl+/+, fl+/−and fl −/− mice. (FIG. 6C) DNA PCR gel image showing deletion of miR-6236 DNA locus between inserted loxP sites by TAT-Cre recombinase treatment in vitro in BMDM of fl+/+ mice. (FIG. 6D) Body weight of wild type (LysM −/−) and myeloid specific knockout (LysM+/−) male mice before and after DIO feeding (n=5-7). (FIG. 6E) Body weight of LysM −/− and LysM+/−female mice before and after DIO feeding (n=4). (FIG. 6F) Fed and fasted (5 hrs.) blood glucose level in DIO LysM+/−and LysM −/− female mice (n=4). (FIG. 6G) Fed and fasted (5 hrs.) serum insulin level in DIO LysM+/−and LysM −/− female mice (n=4). (FIG. 6H) miR-6236 expression in EVs of BMDMs derived from miR-6236^{fl/fl} mice. nd=not detected. Data representative of ≥2 experiments. Data presented as ‘mean±SEM’; * p<0.05, ** p<0.01, *** p<0.001, Student′s t test.

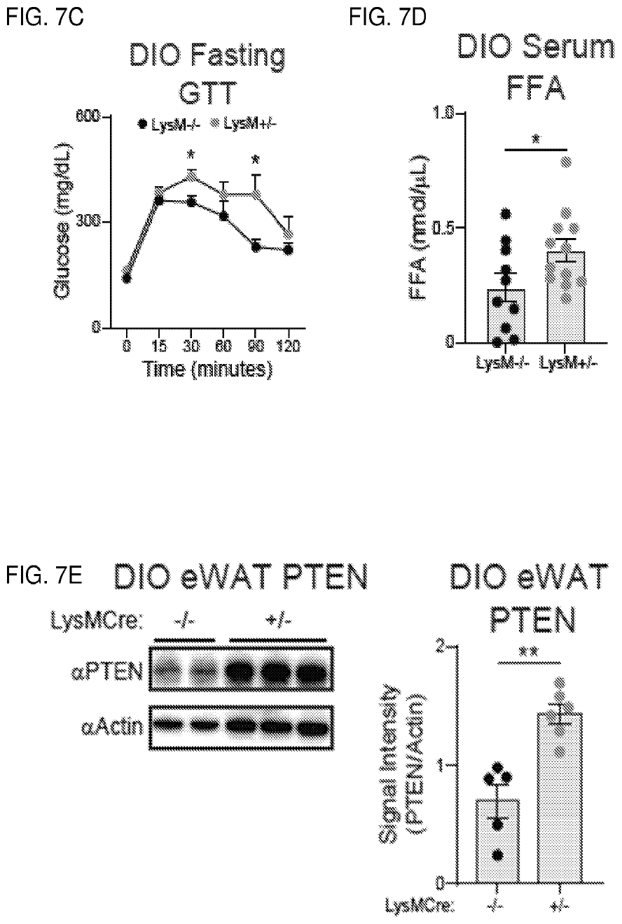

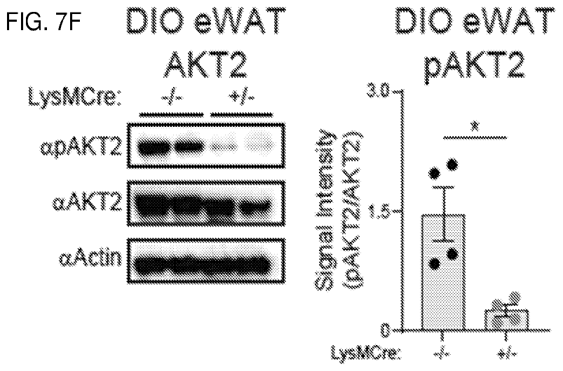

FIGS. 7A-7F. Myeloid specific deletion of miR-6236 exacerbates obesity-associated metabolic outcomes. (FIG. 7A) Fed and fasted (5 hrs.) blood glucose level in DIO wild type (LysM −/−) and myeloid specific knockout (LysM+/−) male mice (n=4). (FIG. 7B) Fed and fasted (5 hrs.) serum insulin level in DIO male mice (n=4). (FIG. 7C) Intraperitoneal glucose tolerance test in DIO male mice (n=4). (FIG. 7D) Serum free fatty acid (FFA) level in DIO male mice (n=10-12). (FIG. 7E) PTEN Western blot and quantification in eWAT of DIO male mice (n=2-3). (FIG. 7F) Akt2 and pAkt2 Western blot and quantification in eWAT of DIO male mice (n=2). Data representative of >2 experiments. Data presented as ‘mean±SEM’; *p<0.05, ** p<0.01, *** p<0.001, Student's t test.

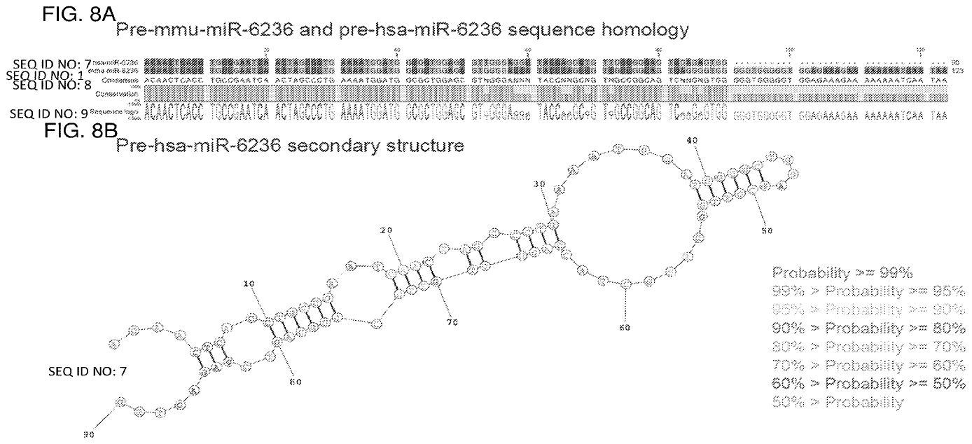

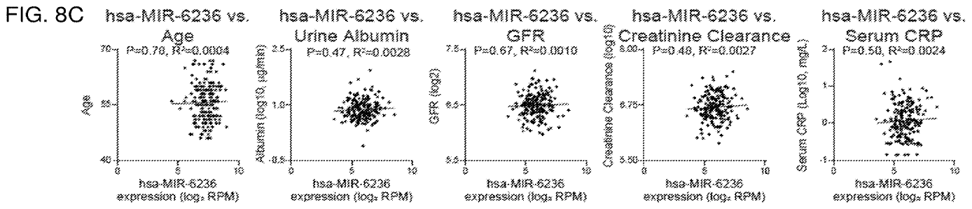

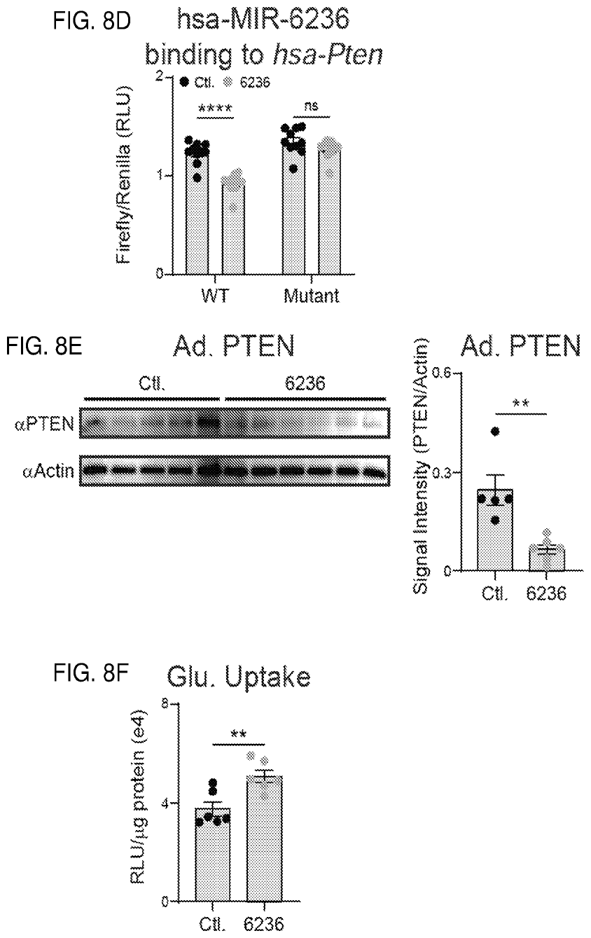

FIGS. 8A-8F. Human and mice miR-6236 sequence homology, stem-loop structure of human miR-6236 and correlation of human miR-6236 with other cardiometabolic traits. (FIG. 8A) Alignment of mice and human miR-6236 precursor sequences. (FIG. 8B) Stem-loop structure of human miR-6236 precursor sequence. (FIG. 8C) Correlation of miR-6236 expression level in subcutaneous adipose tissue with urine albumin level, GFR (glomerular filtration rate), creatinine clearance rate and serum CRP (C-reactive protein) level of the patient (n=189). *p<0.05, ** p<0.01, *** p<0.001, Pearson correlation. (FIG. 8D) Dual luciferase reporter assay of scrambled control RNA (Ctl.) or hsa-MIR-6236 (6236) binding to a wild type (WT) hsa-PTEN 3′UTR or hsa-PTEN 3′UTR with hsa-MIR-6236 binding sites deleted (Mutant) (n=10). (FIG. 8E) PTEN Western blot and quantification in primary human adipocytes (n=5-6). (FIG. 8F) Insulin-stimulated glucose uptake in primary human adipocytes (n=6).

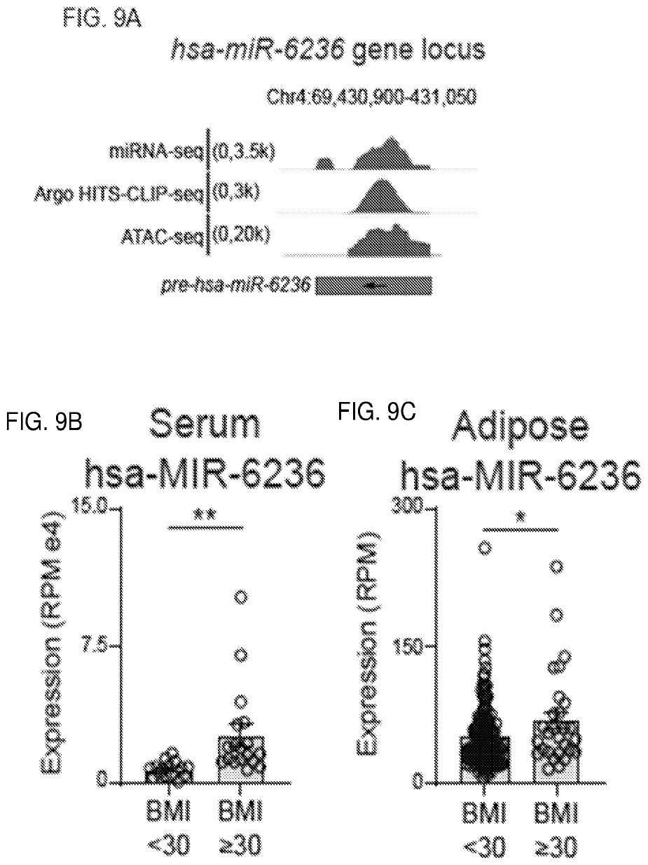

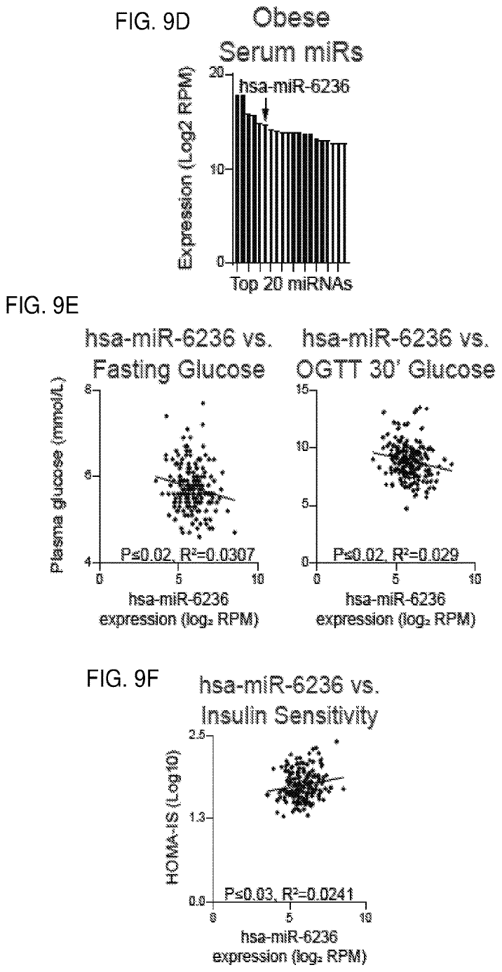

FIGS. 9A-9F. miR-6236 in human is elevated during obesity and correlates with blood glucose level and insulin sensitivity of the patient. (FIG. 9A) Human miR-6236 genomic locus (GRCH38, Chr4: 69430994-69430905; ‘-’strand) and alignment of adipose tissue sequencing reads. (FIG. 9B) MiR-6236 level in serum of lean and obese male subjects measured by small RNA sequencing (n=15-18). RPM=Read Per Million. (FIG. 9C) Expression level miR-6236 in subcutaneous adipose tissue of lean and obese male subjects measured by small RNA sequencing (n=28-162). (FIG. 9D) The 20 most abundant miRNAs in serum of obese male subjects given in panel C. (FIG. 9E) Correlation between miR-6236 expression level in subcutaneous adipose tissue and fasting blood glucose level or OGTT 30-minute blood glucose (n=189). (FIG. 9F)

Correlation between miR-6236 expression level in subcutaneous adipose tissue and patient HOMA-IS score (n=189).Data presented as ‘mean±SEM’; * p<0.05, ** p<0.01, Student′s t test for panel 9B, 9C; Pearson correlation for panel 9E, 9F).

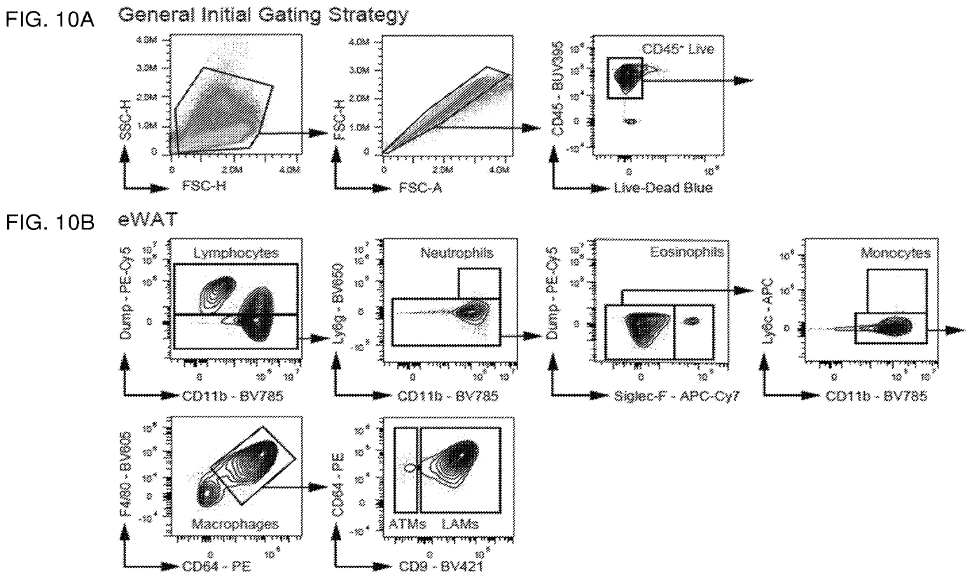

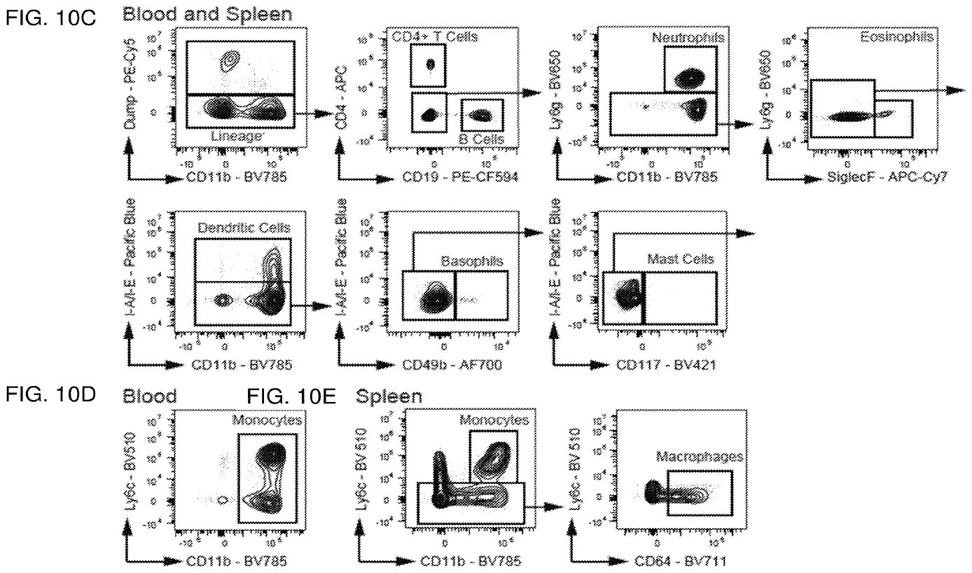

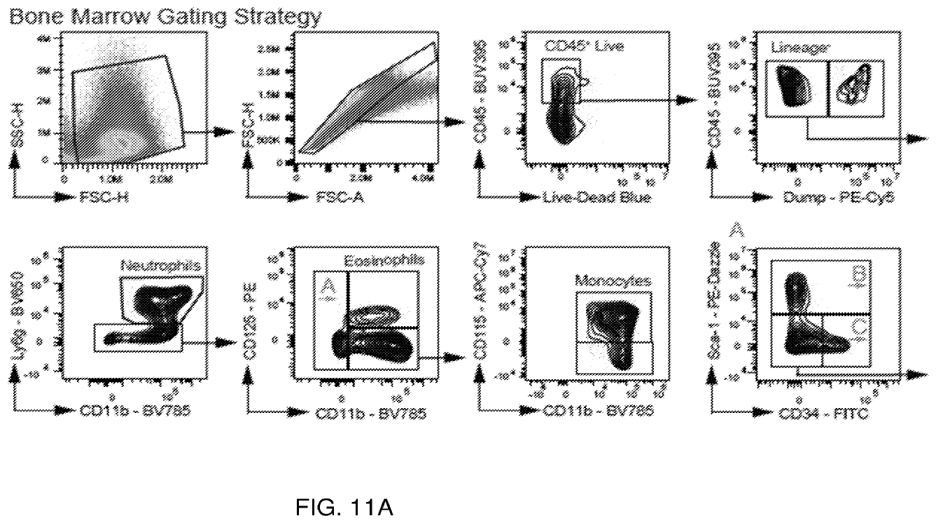

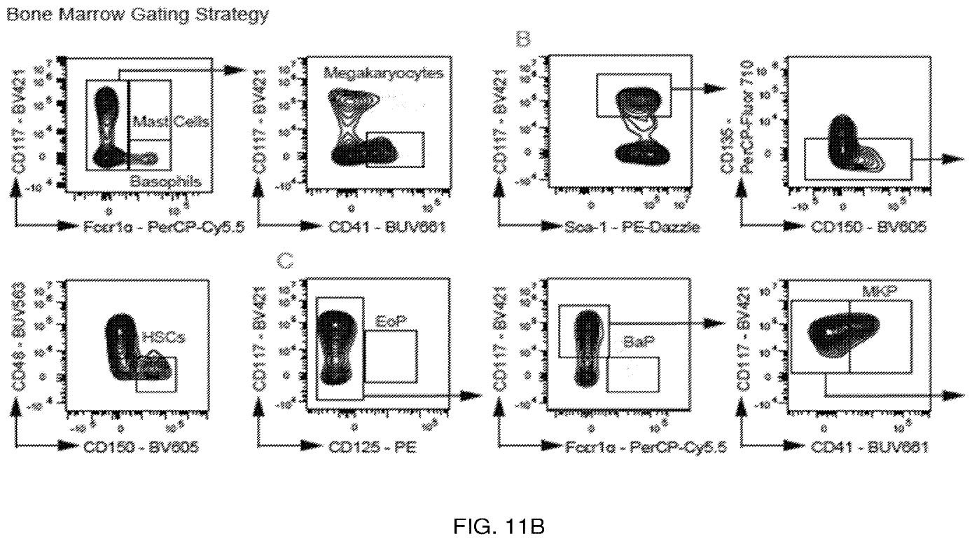

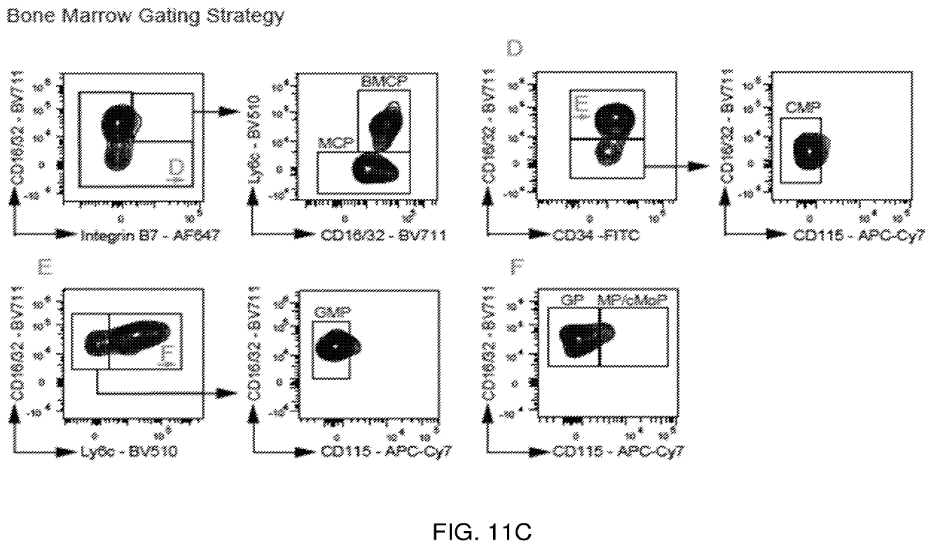

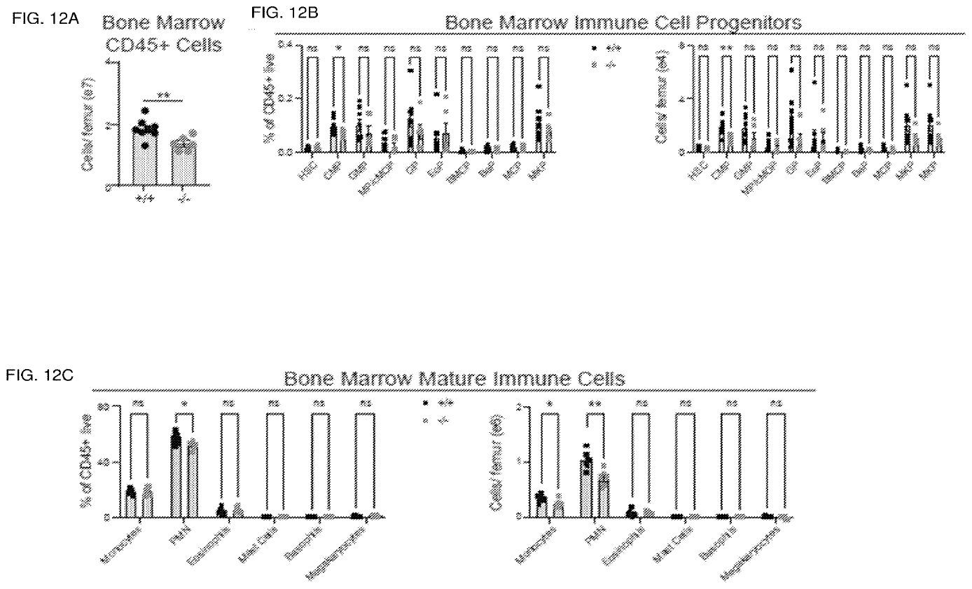

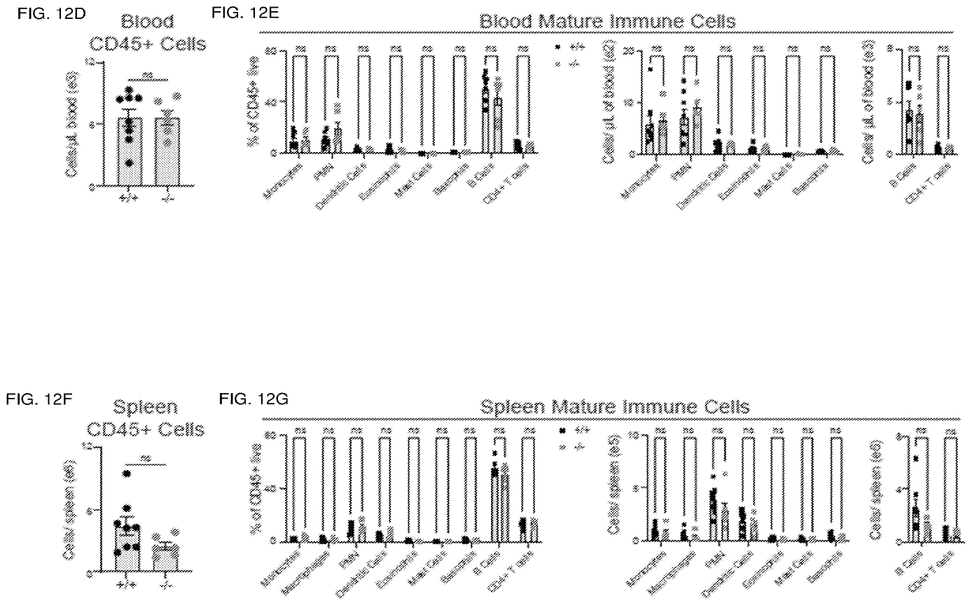

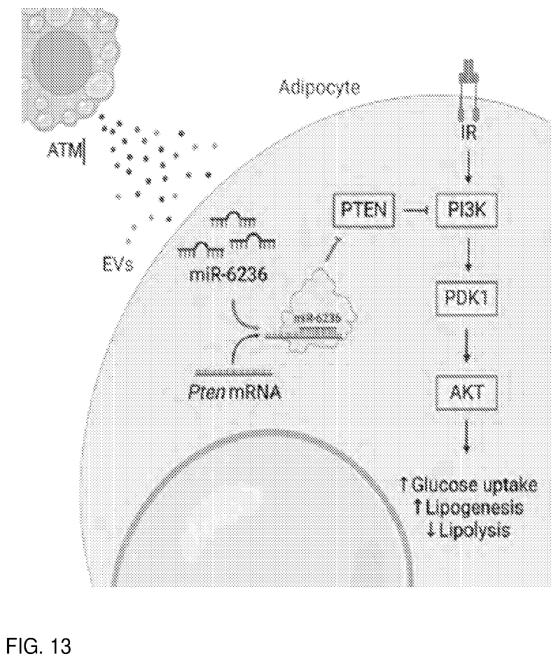

FIGS. 10A-10E. Peripheral gating strategies. General initial (FIG. 10A), eWAT (Dump=CD3, B220, and NK1.1) (FIG. 10B), and blood and spleen (Dump=CD8a, NK1.1, Ter119) (FIGS. 10C-10E) gating strategies.

FIGS. 11A-11C. Bone marrow gating strategy. Dump=CD3, NK1.1, Ter119, B220, and IL7R.

FIG. 12A-12G. Bone marrow, spleen, and blood immune cell complement of DIO wild type or miR-6236 deficient mice. Flow cytometric analysis of CD45+ cells (FIG. 12A), immune cell progenitors (FIG. 12B), and mature immune cells (FIG. 12C) in bone marrow of DIO wild type littermate (+/+) or whole-body miR-6236 knockout (−/−) male mice (n=6-8). Flow cytometric analysis of CD45+ cells (FIG. 12D) and mature immune cells (FIG. 12E) in blood of DIO wild type littermate (+/+) or whole-body miR-6236 knockout (−/−) male mice (n=6-8). Flow cytometric analysis of CD45+ cells (FIG. 12F) and mature immune cells (FIG. 12G) in spleen of DIO wild type littermate (+/+) or whole-body miR-6236 knockout (−/−) male mice (n=6-8). Gating strategies shown in FIGS. 10 & 11. Data representative of >2 experiments. Data presented as mean±SEM; ns=not significant, *p<0.05, ** p<0.01, Student's t test.

FIG. 13. Model. ATMs secrete EVs which deliver miR-6236 to adipocytes. miR-6236 binds to the 3′UTR of the PTEN mRNA, suppressing translation and potentiating insulin signaling. The ultimate effect is improved glucose homeostasis and metabolic outcomes during obesity.

### DETAILED DESCRIPTION OF THE INVENTION

MicroRNA (miRNA) is a single-stranded non-coding RNA involved in the process of post-transcriptional control of gene expression in eukaryotes. A large number of publications reveal that miRNAs are implicated in obesity-related diabetes and CVD. The increase or alteration of miRNAs in exosomes may promote or protect against the occurrence and development of obesity-related diabetes and/or cardiovascular complications. Additionally, some other studies identify that miRNAs in exosomes are likely involved in cardiac regeneration and confer cardiac protective effects. Therefore, targeting miRNAs in exosomes, or mimicking the action of specific miRNAs, is regarded as a potent therapeutic measure to alleviate diabetes-related CVD.

Lipid associated macrophages (LAMs), including adipose tissue macrophages (ATMs) can protect against obesity-associated metabolic dysfunction, but the mechanisms by which they perform this function remain largely unknown. The data presented herein demonstrate that miR-6236 is a bona fide miRNA that is secreted by LAMs during obesity. Moreover, global or myeloid cell-specific deletion of miR-6236 aggravates obesity-associated adipose tissue insulin resistance, hyperglycemia, hyperinsulinemia, and hyperlipidemia. MiR-6236 regulates adipocyte insulin sensitivity by inhibiting translation of Pten, a negative regulator of insulin signaling. The human genome harbors a miR-6236 homolog that is one of the most highly expressed miRs in adipose tissue and serum of obese people. Hsa-miR-6236 expression in adipose tissue negatively correlates with hyperglycemia, and positively correlates with insulin sensitivity, during obesity. Our findings establish miR-6236 and mimics thereof as one mechanism by which LAMs exert their beneficial functions by enhancing the effects of insulin on adipocytes. Our data also establish miR-6236 as an ATM-secreted miRNA that potentiates adipocyte insulin signaling and protects against metabolic dysfunction during obesity. Accordingly, miR-6236 and mimics thereof can be used to advantage as new therapeutics for the treatment of Type 2 Diabetes.

### Definitions

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a”, “or” and “an” can mean “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The terms “agent”, and “test compound” denote a chemical compound, a mixture of chemical compounds, a biological macromolecule, or an extract made from biological materials such as bacteria, plants, fungi, or animal (particularly mammalian) cells or tissues. Biological macromolecules include peptides, peptide/DNA complexes, siRNA, shRNA, antisense oligonucleotides, and any nucleic acid-based molecule which encoded the proteins described herein.

It is also contemplated that the term “compound” or “compounds” refers to the compounds discussed herein and includes precursors and derivatives of the compounds, and pharmaceutically acceptable salts of the compounds, precursors, and derivatives.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g., by manipulation of the nucleic acid encoding the protein or by altering the protein itself. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

For purposes of the invention, “nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated.

“Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

A “specific binding pair” comprises a specific binding member (sbm) and a binding partner (bp) which have a particular specificity for each other and which in normal conditions bind to each other in preference to other molecules. Examples of specific binding pairs are antigens and antibodies, biotin and streptavidin, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long. “Exosomes” are nanovesicles released from a variety of different cells. These small vesicles may be derived from large multivesicular endosomes and secreted into the extracellular milieu. The precise mechanisms of exosome release/shedding remain unclear. They appear to form by invagination and budding from the limiting membrane of late endosomes, resulting in vesicles that contain cytosol and that expose the extracellular domain of membrane-bound cellular proteins on their surface. Using electron microscopy, studies have shown fusion profiles of multivesicular endosomes with the plasma membrane, leading to the secretion of the internal vesicles into the extracellular environment.

The term “therapeutic agent” is art-recognized and refers to any chemical moiety that is a biologically, physiologically, or pharmacologically active substance that acts locally or systemically in a subject. According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “delivery” as used herein refers to the introduction of foreign molecule (i.e., miRNA encoding the polypeptide of interest) into cells. The term “administration” as used herein means the introduction of a foreign molecule into a cell. The term is intended to be synonymous with the term “delivery”.

As used herein, the term “activity” refers to a biological activity.

As used herein, the term “pharmacological activity” refers to the inherent physical properties of a peptide or polypeptide. These properties include but are not limited to half-life, solubility, and stability and other pharmacokinetic properties.

The term “hit” refers to a test compound that shows desired properties in an assay. The term “test compound” refers to a chemical to be tested by one or more screening method(s) as a putative modulator. A test compound can be any chemical, such as an inorganic chemical, an organic chemical, a protein, a peptide, a carbohydrate, a lipid, or a combination thereof. Usually, various predetermined concentrations of test compounds are used for screening, such as 0.01 micromolar, 1 micromolar and 10 micromolar. Test compound controls can include the measurement of a signal in the absence of the test compound or comparison to a compound known to modulate the target.

The terms “high,” “higher,” “increases,” “elevates,” or “elevation” refer to increases above basal levels, e.g., as compared to a control. The terms “low,” “lower,” “reduces,” or “reduction” refer to decreases below basal levels, e.g., as compared to a control.

The term “modulate” as used herein refers to the ability of a compound to change an activity in some measurable way as compared to an appropriate control. As a result of the presence of compounds in the assays, activities can increase or decrease as compared to controls in the absence of these compounds. Preferably, an increase in activity is at least 25%, more preferably at least 50%, most preferably at least 100% compared to the level of activity in the absence of the compound. Similarly, a decrease in activity is preferably at least 25%, more preferably at least 50%, most preferably at least 100% compared to the level of activity in the absence of the compound. A compound that increases a known activity is an “agonist”. One that decreases, or prevents, a known activity is an “antagonist”.

The term “inhibit” means to reduce or decrease in activity or expression. This can be a complete inhibition or activity or expression, or a partial inhibition. Inhibition can be compared to a control or to a standard level. Inhibition can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64,65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

The term “preventing” as used herein refers to administering a compound prior to the onset of clinical symptoms of a disease or conditions so as to prevent a physical manifestation of aberrations associated with the disease or condition.

The term “in need of treatment” as used herein refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the disclosed compounds.

As used herein, “subject” includes, but is not limited to, animals, plants, bacteria, viruses, parasites and any other organism or entity. The subject can be a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects.

By “treatment” and “treating” is meant the medical management of a subject with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. It is understood that treatment, while intended to cure, ameliorate, stabilize, or prevent a disease, pathological condition, or disorder, need not actually result in the cure, ameliorization, stabilization or prevention. The effects of treatment can be measured or assessed as described herein and as known in the art as is suitable for the disease, pathological condition, or disorder involved. Such measurements and assessments can be made in qualitative and/or quantitative terms. Thus, for example, characteristics or features of a disease, pathological condition, or disorder and/or symptoms of a disease, pathological condition, or disorder can be reduced to any effect or to any amount.

A cell can be in vitro. Alternatively, a cell can be in vivo and can be found in a subject. A “cell” can be a cell from any organism including, but not limited to, a bacterium.

By the term “effective amount” of a compound as provided herein is meant a nontoxic but sufficient amount of the compound to provide the desired result.

By “pharmaceutically acceptable” is meant a material that is not biologically or otherwise undesirable, i.e., the material can be administered to a subject along with the selected compound without causing any undesirable biological effects or interacting in a deleterious manner with any of the other components of the pharmaceutical composition in which it is contained.

I. miRNAs

In certain embodiments of the invention, isolated exosomes or lipid nanovesicles comprising microRNAs (abbreviated miRNAs) may be used in methods and compositions for treating patients at risk for or having one or more diseases related to obesity associated metabolic dysfunction, e.g., cardiovascular disease, diabetes, and hypertension. In particular embodiments, the miRNAs may include miR-6236 and variants thereof.

As noted above, miRNAs are naturally occurring, small non-coding RNAs that are about 17 to about 25 nucleotide bases (nt) in length in their biologically active form. miRNAs post-transcriptionally regulate gene expression by repressing target mRNA translation. It is thought that miRNAs function as negative regulators, i.e. greater amounts of a specific miRNA will correlate with lower levels of target gene expression.

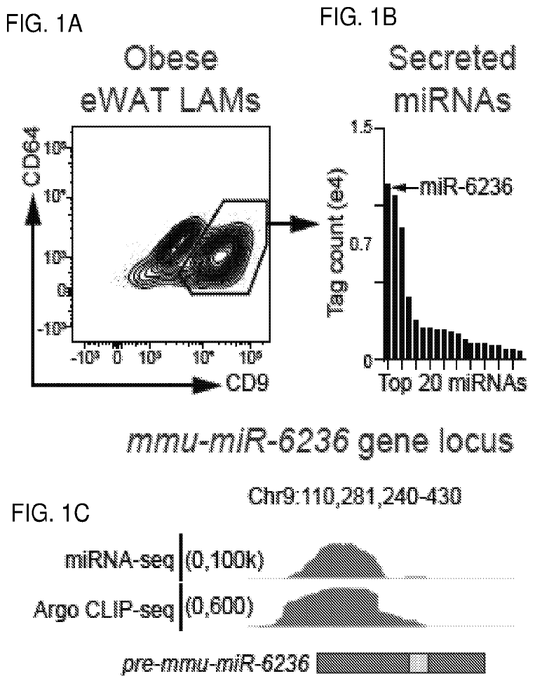

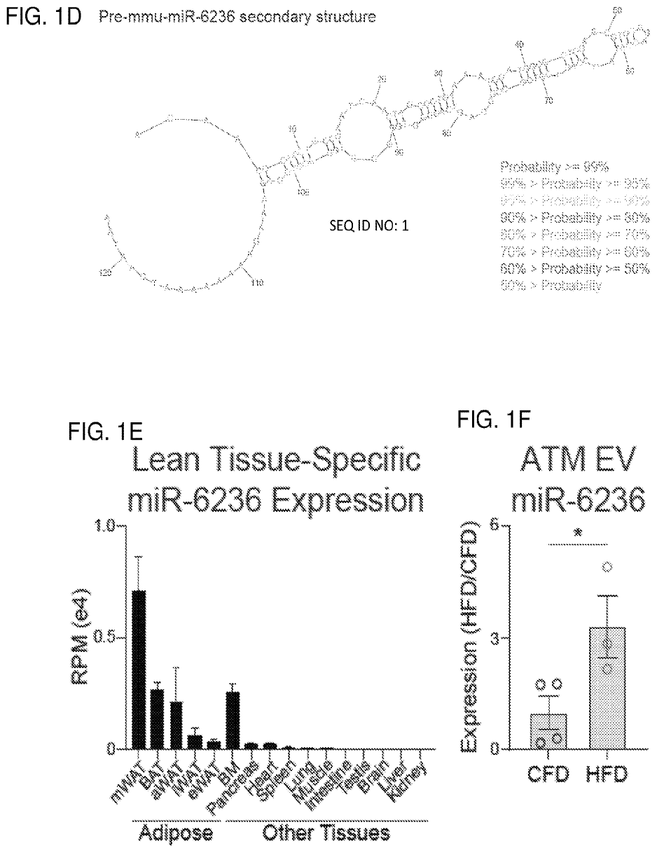

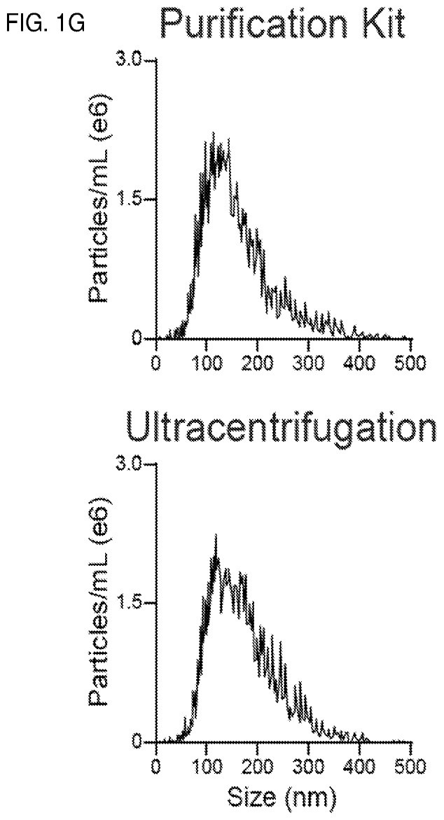

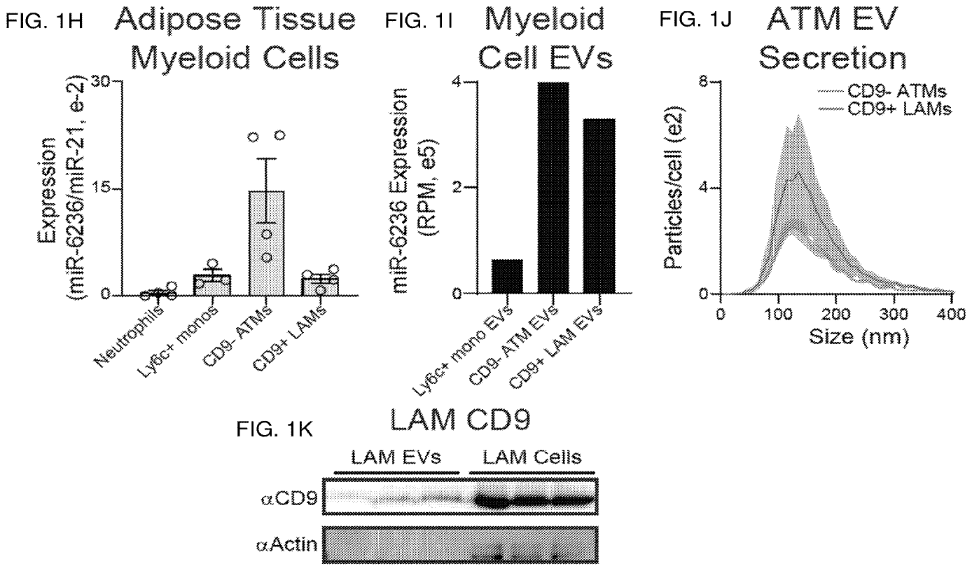

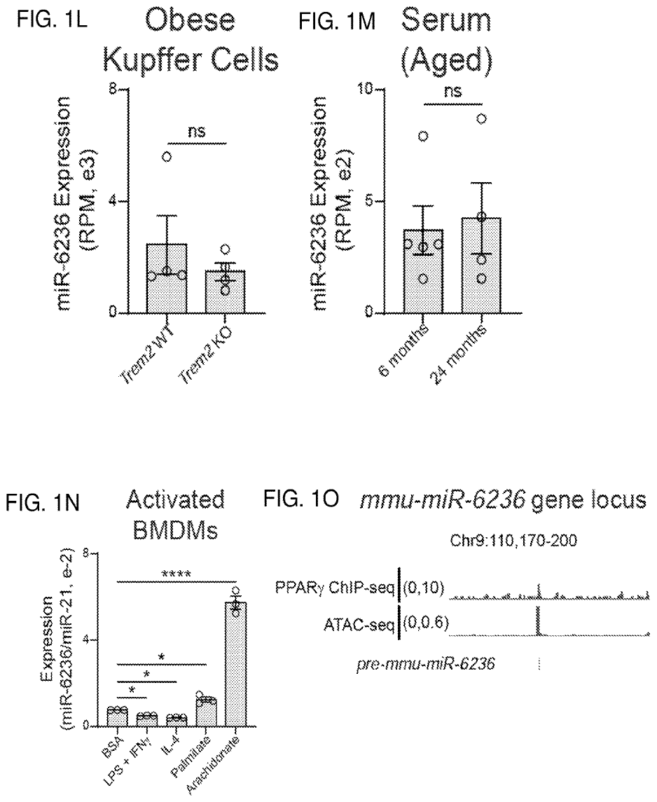

There are three forms of miRNAs existing in vivo, primary miRNAs (pri-miRNAs), premature miRNAs (pre-miRNAs), and mature miRNAs. Primary miRNAs (pri-miRNAs) are expressed as stem-loop structured transcripts of about a few hundred bases to over 1 kb. See FIG. 1D. The pri-miRNA transcripts are cleaved in the nucleus by an RNase II endonuclease called Drosha that cleaves both strands of the stem near the base of the stem loop. Drosha cleaves the RNA duplex with staggered cuts, leaving a 5′ phosphate and 2 nt overhang at the 3′ end.

The cleavage product, the premature miRNA (pre-miRNA) is about 60 to about 110 nt long with a hairpin structure formed in a fold-back manner. Pre-miRNA is transported from the nucleus to the cytoplasm by Ran-GTP and Exportin=5. Pre-miRNAs are processed further in the cytoplasm by another RNase II endonuclease called Dicer. Dicer recognizes the 5′ phosphate and 3′ overhang, and cleaves the loop off at the stem-loop junction to form miRNA duplexes. The miRNA duplex binds to the RNA-induced silencing complex (RISC), where the antisense strand is preferentially degraded and the sense strand mature miRNA directs RISC to its target site. It is the mature miRNA that is the biologically active form of the miRNA and is about 17 to about 25 nt in length.

MicroRNAs function by engaging in base pairing (perfect or imperfect) with specific sequences in their target genes' messages (mRNA). The miRNA degrades or represses translation of the mRNA, causing the target genes' expression to be post-transcriptionally downregulated, repressed, or silenced. In animals, miRNAs do not necessarily have perfect homologies to their target sites, and partial homologies lead to translational repression, whereas in plants, where miRNAs tend to show complete homologies to the target sites, degradation of the message (mRNA) prevails.

MicroRNAs are widely distributed in the genome, dominate gene regulation, and actively participate in many physiological and pathological processes. For example, the regulatory modality of certain miRNAs is found to control cell proliferation, differentiation, and apoptosis; and abnormal miRNA profiles are associated with oncogenesis. Additionally, it is suggested that viral infection causes an increase in miRNAs targeted to silence “pro-cell survival” genes, and a decrease in miRNAs repressing genes associated with apoptosis (programmed cell death), thus tilting the balance toward gaining apoptosis signaling.

### II. Exosomes

In certain aspects of the invention, exosomes may be prepared and used as a novel therapeutic modality for the treatment of patients at risk for or having one or more diseases related to obesity associated metabolic dysfunction.

Exosomes are small membrane vesicles of endocytic origin that are secreted by many cell types. For example, exosomes may have a diameter of about 40 to about 100 nm. They may be formed by inward budding of the late endosome leading to the formation of vesicle-containing multivesicular bodies (MVB) which then fuse with the plasma membrane to release exosomes into the extracellular environment. Though their exact composition and content depends on cell type and disease state, exosomes all share certain characteristics.

In certain aspects, the exosomes may be purified by ultracentrifugation in a sucrose gradient, then identified by the presence of marker proteins such as Alix and CD63 (Schorey & Bhatnagar, 2008) or enrichment of tetraspanins and heat shock protein 70 (Lee, et al., 2011), all of which are specifically expressed on exosomes. Furthermore, exosomes can be isolated in vivo from malignant effusions and normal body fluids such as urine, blood, and cerebrospinal fluid, making them a promising source of diagnostic biomarkers. In some other aspects, exosomes can be isolated using isolation kits, such as those described below and exoEasy Maxi Kit from Qiagen, SmartSEC™.

Exosomes also have the potential for directional homing to specific target cells, depending on the physical properties of their membranes. Their effect can be local, regional or systemic. Exosomes do not contain a random sampling of their parent cell's cytoplasm, but are enriched in specific mRNA, miRNA and proteins (Bobrie, et al., 2011). This cargo is protected from degradation by proteases and RNases while the vesicle is in the interstitial space, and retains bioactivity once taken up by a recipient cell. In this way, they facilitate the transfer of interactive signaling and enzymatic activities that would otherwise be restricted to individual cells based on gene expression (Lee, et al., 2011). For example, Skog and coworkers show that mRNA for a reporter protein can be incorporated into exosomes, transferred to a recipient cell, and translated (Skog, et al., 2008).

The exosomes produced or released by cells may be isolated and/or purified using several techniques. These include filtration, centrifugation, ion-chromatography, or concentration, either alone or in combinations. An exemplary purification method comprises a step of density gradient centrifugation. Another exemplary method comprises a step of ultrafiltration, either alone or coupled to a centrifugation step.

Selective purification or enrichment of physiologically active subpopulations of exosomes may be achieved via several procedures. In certain embodiments, effective exosomes may be concentrated to an enriched sample via use of specific surface protein markers and related separation techniques. In other embodiments, effective exosomes may be harvested from enriched primary cells cultures identified as capable of producing the effective exosomes. In further embodiments, based on screening procedures used to identify candidate effective exosome species, other exosomes may be fabricated using molecular engineering strategies designed to selectively produce exosomes containing the target (i.e., postulated) therapeutic molecular species. The latter may be confirmed by application of exosomes containing fabricated species to naïve cultures, where the desired effect (e.g., increased myelination) may be verified.

In certain embodiments, the exosomes or vesicles may be loaded with therapeutic agents such as nucleic acid molecules. The methods may include, but are not limited to:
(a) Electroporation. By this method, a number of holes are made in cells/exosomes by briefly shocking them with an electric field of 100-200 V/cm. The DNA/RNA can enter the cells/exosomes through the holes made by the electric field.
(b) Lipofection. The method commonly called transfection and can be used to transform cells/exosomes with DNA/RNA via vesicles containing the desired genetic constructs. The vesicles fuse with the cell membrane (similar to how two oil spots at the top of a broth will fuse) and the contents of the vesicles and the cells are combined. There are a number of transfection kits in the market, ready for use, e.g. DeliverX siRNA Transfection Kit (cat. No. DX0002) from Panomics, FuGENE® HD Transfection Reagent (Cat. no. 04709691001) from Roche and LIPOFECTAMINE™ 2000 (Cat. No. 11668-027) from Invitrogen.
(c) Transformation using heat shock. Chilling cells/exosomes in the presence of divalent cations such as Ca²⁺ (in CaCl₂)) makes their membranes become permeable to RNA or DNA plasmids or fragments. Cells or exosomes are incubated with the DNA and then briefly heat shocked (42° C. for 30-120 seconds), which causes the DNA to enter the cell. This method may work well for condensed circular plasmid DNAs and may work for exosomal or lipid nanovesicle constituents.

The above methods describe briefly how production and delivery of modified exosomes can be achieved to transfer RNA and DNA to recipient cells. Exosomes can be engineered to contain RNA/DNA or modified to contain the gene of interest and may be isolated and shifted to the recipient cells, to affect their biological function or survival. Consequently, the exosomes may dispose their content into the cytoplasm of the target cells, which in turn leads to translation of mRNA to specific proteins in the target cell. Further, exosomes are capable of carrying and transferring small coding and non-coding RNA such as microRNA and siRNA that may regulate translation of a specific gene.

Modified or loaded exosomes being vesicles as carriers of DNA or RNA as described herein can be used to treat inherited diseases in targeted cell types and organs. Modified or loaded exosome vesicles can also be used as carriers of DNA or RNA constructs for treatments of patients at risk for or having one or more diseases related to obesity associated metabolic dysfunction disorders, including but not limited to diabetes, cardiovascular disease, and hypertension, or for transfer through any biological membrane.

Changing or modifying the genetic material of exosomes by altering the conditions for the exosome-producing cells is achieved by changing pH, temperature, growing conditions, or using antibodies/chemicals toward exosome-producing cells. This results in alteration of the nucleic acid content.

To administer nucleic acids to recipient cells or tissues, DNA or RNA-containing exosomes can be administered to cells by addition of the exosomes to cell cultures in vitro, or injection of these exosomes intravenously, or by any other route, in vivo as is known in the art, such as nasally or intravenously. Exosomes can be targeted to any cell in the body, including cells in the cardiovascular system, skeletal muscle cells, joint cells, neural cells, gut cells, lung cells, liver cells or kidney cells, or cells in the immune system, or to any type of cell with any function or dysfunction in the body of humans or animals, including malignant cells. As disclosed in the invention herein, exosomes can be used to deliver genetic material to recipient cells to produce any drug or precursor of any drug, or to affect the function or metabolism of any drug, in any cell in humans or animals.

### III. Diseases

Diseases to be prevented, treated or diagnosed can be any disease that affects a subject that would be amenable to therapy or prevention through administration of a composition or a method as described herein. For example, the disease may be related to obesity, including without limitation cardiovascular disease, diabetes, and hypertension. In particular examples, there may be provided methods and compositions involving administering compositions involving isolated exosomes from cells that contain molecules useful for the treatment of such diseases.

Non-limiting examples of obesity related disorders, include without limitation, diabetes, hypertension, heart attack, stroke, heart failure, arrythmia, digestive problems, sleep apnea, osteoarthritis, and certain cancers.

### IV. Therapeutic Agents or Diagnostic Agents for Exosomes

In some embodiments, therapeutic agents or diagnostic agents may be loaded to the exosomes for delivery to a subject, such as by electroporation or other method known in the art. The therapeutic agents may be a therapeutic nucleic acid, a protein or antibody fragment, or a small molecule.

A “therapeutic nucleic acid” is defined herein to refer to a nucleic acid which can be administered to a subject for the purpose of treating or preventing a disease. The nucleic acid is one which is known to be of benefit in the treatment of a disease or health-related condition in a subject.

Therapeutic benefit may arise, for example, as a result of alteration of expression of a particular gene or genes by the nucleic acid. Alteration of expression of a particular gene (e.g PTEN) or genes may be inhibition or augmentation of expression of a particular gene (e.g., via miRNA). In certain embodiments, the therapeutic nucleic acid can encode one or more proteins or polypeptides that can be applied in the treatment or prevention of a disease or health-related condition in a subject (i.e., via mRNA). The terms “protein” and “polypeptide” are used interchangeably herein. Both terms refer to an amino acid sequence comprising two or more amino acid residues.

Any nucleic acid known to those of ordinary skill in the art that is known or suspected to be of benefit in the treatment or prevention of a disease or health-related condition is contemplated in certain aspects as a therapeutic nucleic acid. The phrase “nucleic acid sequence encoding,” as set forth throughout this application, refers to a nucleic acid which directs the expression of a specific protein or peptide. The nucleic acid sequences include both the DNA strand sequence that is transcribed into RNA and the RNA sequence that is translated into protein. In some embodiments, the nucleic acid includes a therapeutic gene. The term “gene” is used to refer to a nucleic acid sequence that encodes a functional protein, polypeptide, or peptide-encoding unit.

As will be understood by those in the art, the term “therapeutic nucleic acid” includes genomic sequences, cDNA sequences, and smaller engineered gene segments that express, or may be adapted to express, proteins, polypeptides, domains, peptides, fusion proteins, and mutants. The nucleic acid may comprise a contiguous nucleic acid sequence of about 5 to about 12000 or more nucleotides, nucleosides, or base pairs.

Encompassed within the definition of “therapeutic nucleic acid” is a “biologically functional equivalent” of a therapeutic nucleic acid that has proved to be of benefit in the treatment or prevention of a disease or health-related condition. Accordingly, sequences that have about 70% to about 99% homology to a known nucleic acid are contemplated in certain aspects.

In certain approaches, the therapeutic nucleic acid can be stabilized. A stabilized nucleic acid molecule is a nucleic acid molecule, preferably an RNA molecule that is modified such, that it is more stable to disintegration or degradation, e.g., by environmental factors or enzymatic digest, such as by an exo- or endonuclease degradation, than the nucleic acid molecule without the modification. Preferably, a stabilized nucleic acid molecule in the context of the present invention is stabilized in a cell, such as a prokaryotic or eukaryotic cell, preferably in a mammalian cell, such as a human cell. The stabilization effect may also be exerted outside of cells, e.g. in a buffer solution etc., for example, in a manufacturing process for a pharmaceutical composition comprising the stabilized nucleic acid molecule.

A 5′-cap is an entity, typically a modified nucleotide entity, which generally “caps” the 5′-end of a mature mRNA or miRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. Further examples of 5′ cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′ phosphorothioate, phosphorodithioate, or bridging or non-bridging methylphosphonate moiety.

The phosphate backbone may also be modified in the modified nucleosides and nucleotides, which may be incorporated into a modified RNA as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

The modified nucleosides and nucleotides, which may be incorporated into a modified RNA as described herein can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-5-chloropurineriboside-5′-triphosphate, 2-Aminopurine-riboside-5′-triphosphate; 2-aminoadenosine-5′-triphosphate, 2′-Amino-2′-deoxycytidine-triphosphate, 2-thiocytidine-5′-triphosphate, 2-thiouridine-5′-triphosphate, 2′-Fluorothymidine-5′-triphosphate, 2′-O-Methyl-inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2-deoxycytidine-5′-triphosphate, 5-Bromo-2-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2-deoxyuridine-5′-triphosphate, 5-methylcytidine-5′-triphosphate, 5-methyluridine-5′-triphosphate, 5-Propynyl-2-deoxycytidine-5′-triphosphate, 5-Propynyl-2-deoxyuridine-5′-triphosphate, 5-azacytidine-5′-triphosphate, 6-azauridine-5′-triphosphate, 5-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin=5′-triphosphate, xanthosine-5′-triphosphate. Particular preference is given to nucleotides for base modifications selected from the group of base-modified nucleotides consisting of 5-methylcytidine-5″-triphosphate, 7-deazaguanosine-5″-triphosphate, 5-bromocytidine-5′-triphosphate, and pseudouridine-5′-triphosphate.

In some embodiments, modified nucleosides include pyridin=4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,5-diaminopurine, 7-deaza-8-aza-2,5-diaminopurine, 1-methyladenosine, N6-methyladenosine, N6-isopentenyladenosine, N6-(cis-hydroxyisopentenyl) adenosine, 2-methylthio-N5-(cis-hydroxyisopentenyl) adenosine, N6-glycinylcarbamoyladenosine, N6-threonylcarbamoyladenosine, 2-methylthio-N6-threonyl carbamoyladenosine, 115,115-dimethyladenosine, 7-methyladenine, 2-methylthio-adenine, and 2-methoxy-adenine. In other embodiments, modified nucleosides include inosine, 1-methyl-inosine, wyosine, wybutosine, 7-deaza-guanosine, 7-deaza-8-aza-guanosine, 5-thio-guanosine, 5-thio-7-deaza-guanosine, 5-thio-7-deaza-8-aza-guanosine, 7-methyl-guanosine, 5-thio-7-methyl-guanosine, 7-methylinosine, 5-methoxy-guanosine, 1-methylguanosine, N2-methylguanosine, N2,N2-dimethylguanosine, 8-oxo-guanosine, 7-methyl-8-oxo-guanosine, 1-methyl-5-thio-guanosine, N2-methyl-5-thio-guanosine, and N2,N2-dimethyl-5-thio-guanosine.

In some embodiments, the nucleotide can be modified on the major groove face and can include replacing hydrogen on C-5 of uracil with a methyl group or a halo group. In specific embodiments, a modified nucleoside is 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine.

In further specific embodiments, a modified RNA may comprise nucleoside modifications selected from 5-aza-cytidine, 2-thio-cytidine, α-thio-cytidine, Pseudo-iso-cytidine, 5-aminoallyl-uridine, 5-iodo-uridine, N1-methyl-pseudouridine, 5,5-dihydrouridine, α-thio-uridine, 4-thio-uridine, 5-aza-uridine, 5-hydroxy-uridine, deoxy-thymidine, 5-methyl-uridine, Pyrrolo-cytidine, inosine, α-thio-guanosine, 5-methyl-guanosine, 5-methyl-cytidine, 8-oxo-guanosine, 7-deaza-guanosine, N1-methyl-adenosine, 2-amino-6-Chloro-purine, N6-methyl-2-amino-purine, Pseudo-iso-cytidine, 6-Chloro-purine, N6-methyl-adenosine, α-thio-adenosine, 8-azido-adenosine, 7-deaza-adenosine.

### Lipid Modification:

According to a further embodiment, a modified RNA as defined herein can contain a lipid modification. Such a lipid-modified RNA typically comprises an RNA as defined herein. Such a lipid-modified RNA as defined herein typically further comprises at least one linker covalently linked with that RNA, and at least one lipid covalently linked with the respective linker. Alternatively, the lipid-modified RNA comprises at least one RNA as defined herein and at least one (bifunctional) lipid covalently linked (without a linker) with that RNA. According to a third alternative, the lipid-modified RNA comprises an RNA molecule as defined herein, at least one linker covalently linked with that RNA, and at least one lipid covalently linked with the respective linker, and also at least one (bifunctional) lipid covalently linked (without a linker) with that RNA. In this context, it is particularly preferred that the lipid modification is present at the terminal ends of a linear RNA sequence.

### V. Diagnostic Nucleic Acids

The exosomes or vesicles in some aspects may include a nucleic acid that is a diagnostic or biomarker nucleic acid. A “diagnostic nucleic acid” or “biomarker is a nucleic acid that can be applied in the diagnosis of a disease or health-related condition. In certain embodiments, miR6236 and variants thereof can be employed as diagnostic or prognostic reagents to assess risk of obesity related disorders. Thus, in certain approaches, miRNA6236 could be used to advantage as a biomarker for prognostication, (e.g., higher levels of miR-6236 would be associated with better outcomes, while lower levels would be correlated with great risk of disease). For these purposes, biological samples could be obtained from the subject and levels of miRNA6236 as free RNA or encapsulated within endosomes could be determined.

Also included in the definition of “diagnostic nucleic acid” is a nucleic acid sequence that encodes one or more reporter proteins for research purposes. A “reporter protein” refers to an amino acid sequence that, when present in a cell or tissue, is detectable and distinguishable from other genetic sequences or encoded polypeptides present in cells. In some embodiments, a therapeutic gene may be fused to the reporter or be produced as a separate protein. For example, the gene of interest and reporter may be induced by separate promoters in separate delivery vehicles by co-transfection (co-infection) or by separate promoters in the same delivery vehicle. In addition, the two genes may be linked to the same promoter by, for example, an internal ribosome entry site, or a bi-directional promoter. Using such techniques, expression of the gene of interest and reporter correlate. Thus, one may gauge the location, amount, and duration of expression of a gene of interest. The gene of interest may, for example, be an anti-cancer gene, such as a tumor suppressor gene or pro-apoptotic gene.

In some embodiments, a reporter sequence encodes a fluorescent protein. Examples of fluorescent proteins which may be used in accord with the invention include green fluorescent protein (GFP), enhanced green fluorescent protein (EGFP), Renilla reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED). It is to be understood that these examples of fluorescent proteins is not exclusive and may encompass later developed fluorescent proteins, such as any fluorescent protein within the infrared, visible or ultraviolet spectra.

In various embodiments, the desired level of expression of at least one of the reporter sequences is an increase, a decrease, or no change in the level of expression of the reporter sequence as compared to the basal transcription level of the diagnostic nucleic acid. In a particular embodiment, the desired level of expression of one of the reporter sequences is an increase in the level of expression of the reporter sequence as compared to the basal transcription level of the reporter sequence.

In various embodiments, the reporter sequence encodes unique detectable proteins which can be analyzed independently, simultaneously, or independently and simultaneously. In other embodiments, the host cell may be a eukaryotic cell or a prokaryotic cell. Exemplary eukaryotic cells include yeast and mammalian cells. Mammalian cells include human cells and various cells displaying a pathologic phenotype, such as cancer cells.

### VI. Pharmaceutical Compositions

In certain aspects, the compositions or agents for use in the methods are suitably contained in a pharmaceutically acceptable carrier. The carrier is non-toxic, biocompatible and is selected so as not to detrimentally affect the biological activity of the agent. The agents in some aspects of the invention may be formulated into preparations for local delivery (i.e., to a specific location of the body, such as skeletal muscle or other tissue) or systemic delivery, in solid, semi-solid, gel, liquid or gaseous forms such as tablets, capsules, powders, granules, ointments, solutions, depositories, inhalants and injections allowing for oral, parenteral or surgical administration. Certain aspects of the invention also contemplate local administration of the compositions by coating medical devices and the like.

Suitable carriers for parenteral delivery via injectable, infusion or irrigation and topical delivery include distilled water, physiological phosphate-buffered saline, normal or lactated Ringer's solutions, dextrose solution, Hank's solution, or propanediol. In addition, sterile, fixed oils may be employed as a solvent or suspending medium. For this purpose, any biocompatible oil may be employed including synthetic mono- or diglycerides. In addition, fatty acids such as oleic acid find use in the preparation of injectables. The carrier and agent may be compounded as a liquid, suspension, polymerizable or non-polymerizable gel, paste or salve.

The carrier may also comprise a delivery vehicle to sustain (i.e., extend, delay or regulate) the delivery of the agent(s) or to enhance the delivery, uptake, stability or pharmacokinetics of the therapeutic agent(s). Such a delivery vehicle may include, by way of non-limiting examples, microparticles, microspheres, nanospheres or nanoparticles composed of proteins, liposomes, carbohydrates, synthetic organic compounds, inorganic compounds, polymeric or copolymeric hydrogels and polymeric micelles.

In certain aspects, the actual dosage amount of a composition administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active agent, such as an isolated exosome, a related lipid nanovesicle, or an exosome or nanovesicle loaded with therapeutic agents or diagnostic agents. In other embodiments, the active agent may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 microgram/kg/body weight to about 100 mg/kg/body weight, about 5 microgram/kg/body weight to about 500 milligram/kg/body weight, etc., can be administered.

Solutions of pharmaceutical compositions can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms. In certain aspects, the pharmaceutical compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain less, than, equal to, or more than 10 mg, 25 mg, 50 mg or up to about 100 mg of human serum albumin per milliliter of phosphate buffered saline. Other pharmaceutically acceptable carriers include aqueous solutions, non-toxic excipients, including salts, preservatives, buffers and the like.

Examples of non-aqueous solvents are propylene glycol, polyethylene glycol, vegetable oil and injectable organic esters such as ethyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-fungal agents, anti-oxidants, chelating agents and inert gases.

The pH and exact concentration of the various components the pharmaceutical composition are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders.

In further aspects, the pharmaceutical compositions may include classic pharmaceutical preparations. Administration of pharmaceutical compositions according to certain aspects may be via any common route so long as the target tissue is available via that route. This may include oral, nasal, buccal, rectal, vaginal or topical. Topical administration may be particularly advantageous for the treatment of skin cancers, to prevent chemotherapy-induced alopecia or other dermal hyperproliferative disorder. Alternatively, administration may be by orthotopic, intradermal, subcutaneous, intramuscular, intraperitoneal or intravenous injection. Such compositions would normally be administered as pharmaceutically acceptable compositions that include physiologically acceptable carriers, buffers or other excipients. For treatment of conditions of the lungs, aerosol delivery can be used. Volume of the aerosol is between about 0.01 ml and 0.5 ml.

An effective amount of the pharmaceutical composition is determined based on the intended goal. The term “unit dose” or “dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

Precise amounts of the pharmaceutical composition also depend on the judgment of the practitioner and are peculiar to each individual. Factors affecting the dose include the physical and clinical state of the patient, the route of administration, the intended goal of treatment (e.g., alleviation of symptoms versus cure) and the potency, stability and toxicity of the particular therapeutic substance.

The following materials and methods are provided to facilitate the practice of the present invention.

Generation of miR-6236 Mouse Lines

miR-6236 KO and LoxP mouse lines were generated on a C57BL/6J (B6) background by the University of Pennsylvania CRISPR-Cas9 Mouse Targeting Core. Genomic modifications were confirmed by sanger sequencing. All experiments were performed on experimental mice or appropriate age and sex matched littermate wild-type controls.

### Dietary Treatments of Mice

Mice were generally housed under specific pathogen free conditions with standard bedding and automated running water supply under 12-hr. light/dark cycle. All mice were fed a normal chow diet until the age of 6 weeks, after which obesity was induced by feeding a high caloric diet (Research Diets D12492, 60 Kcal % fat) ad libitum from 6 to 18-24 weeks (Hill et al., 2018). Where indicated, mice were maintained on a control fat diet (CFD, Research Diets D12450B, 10% kcal fat) from 6-18 weeks. Male mice were preferred due to known susceptibility to diet-induce obesity and sequela, though key observations were tested in female mice.

### Fed and Fasting Blood Glucose Measurement

Blood was collected from the tail vein. Glucose was measured using Blood Glucose Strips (Clarity Diagnostics) under fed and brief fasting (~5 hours) conditions.

### Intraperitoneal Glucose Tolerance Test

The day before the experiment, mice were fasted overnight (~12-16 hours) in a clean cage with alpha dry bedding. On the day of experiment mice were weighed and given an intraperitoneal injection of glucose solution at the dose of 2 grams of glucose/kg body weight. Blood glucose levels were measured before (0 min.) and after (15, 30, 60, 90, 120 min.) glucose administration as above.

### Collection of Serum and Tissues

Mice were euthanized by CO₂ asphyxiation. Serum was isolated by allowing blood samples to coagulate at room temperature for ~30 min. followed by centrifugation (1000×g for 10 min.) and storage at −80° C. until future use. Major metabolic organs and tissues were dissected, weighed, and immediately used or flash frozen in liquid nitrogen and stored at −80° C. until future use.

### Measurement of Metabolites in Serum and Tissue

Tissue lysates were prepared from frozen samples in suitable lysis buffers depending on the metabolite to be analyzed and kit to be used. Tissue was homogenized using Bead Rupture Elite homogenizer (OMNI International). The following kits were used to measure their respective metabolite in serum or tissue lysate following the manufacturer's instructions: Free Fatty Acid Assay Kit (Abcam), Free Glycerol Assay Kit (Abcam), Triglyceride Assay Kit (Abcam), and Cholesterol Assay Kit-HDL and LDL/VLDL (Abcam). Metabolite level was normalized to the protein content of the lysate.

### Ex Vivo Glucose Uptake

eWAT was cut into 1-2 mm pieces and placed into digestion media (DMEM with 1% BSA, 6.2 mg/ml Collagenase Type I, and 2.4 mg/ml Dispase II). Tissue was homogenized using a Gentlemacs (Miltenyi Biotech) and adipocytes were separated from stromal vascular fraction (SVF) by centrifugation. Purified adipocytes were resuspended in DMEM with 10% FBS and 3 μg/ml insulin and incubated for ~30 min. Cells were centrifuged, and the adipocyte layer was collected in a cell culture plate. Glucose uptake was performed on insulin-stimulated adipocytes using Glucose Uptake-Glo™ Assay kit (Promega).

### Ex Vivo Lipolysis Assay

Ex vivo lipolysis assay was performed as described previously (Sostre-Colón et al., 2021), with some modifications. Briefly, eWAT was excised, weighed, and placed into DMEM with 2% BSA. The tissue was cut into ~5-7 equal pieces and distributed into fresh 1 ml DMEM with 2% BSA for preincubation (~20 min.). Tissue pieces were subsequently transferred to fresh 1 ml DMEM with 2% BSA and 3 μg/ml insulin. After 20 minutes, tissue pieces were transferred to fresh 1 ml DMEM media with 2% BSA and 3 μg/ml insulin for 60 minutes and the assay media was collected. Glycerol content in the media was measured using Free Glycerol Assay Kit (Abcam) and normalized to tissue weight.

### Insulin and Glucagon ELISA

Serum insulin and glucagon levels were measured in 2 μl of serum sample using Ultra-Sensitive Mouse Insulin ELISA kit (Crystal chem) and Mouse Glucagon ELISA Kit (Crystal Chem) respectively.

### Flow Cytometry and Cell Sorting

Blood was collected from euthanized mice via eye bleeding into blood collection tubes with 10.8 mg EDTA (Fisher Scientific). Blood was treated with RBC Lysis Buffer (Tonbo Biosciences) and filtered through a 70 μM filter.

Spleen samples were passed through a 70 μM filter. Femurs from euthanized mice were dissected and flushed with cold PBS and passed through a 70 μM filter. eWAT samples were finely minced and digested in DMEM with 1 mg/mL Collagenase type IV and 10 mg/mL DNase I for 30 minutes at 37° C. in a shaking incubator. Cells were filtered through a 100 μM filter and adipocyte fraction was removed following centrifugation. All samples were treated with RBC Lysis Buffer (Tonbo Biosciences), washed in MACS buffer (1 ×PBS, 2% fetal bovine serum, 2 mM EDTA, 25 mM HEPES), and Fc-receptor blocked (except bone marrow samples) with CD16/32 (2.4G2, 1:50, BD) prior to staining for flow cytometry. Cells were stained with anti-mouse fluorochrome-conjugated monoclonal antibodies, quantified using an Aurora flow cytometer (Cytek), and analyzed using FlowJo (BD).

All antibodies were used at 1:500 dilution unless noted below. For eWAT samples the following antibodies were used: CD45.2-BUV395, Live/Dead Blue (1:2400), CD9-BV421, F4/80-BV605, Ly6g-BV650, CD11b-BV785, BODIPY 493/503 (1:2400), CD64-PE, I-A/I-E-PE/Dazzle 594, CD11c-PE-Cy5.5, Ly6c-APC, and SiglecF-APC-Cy7. For blood and spleen samples the following antibodies were used Live/Dead Blue (1:2400), CD45-BUV 395, CD11c-BUV737, CD117-BV421, I-A/I-E-Pacific Blue, Ly6c-BV510, F4/80-BV605, Ly6g-BV650, CD64-BV711, CD11b-BV785, Fcerla-PerCP-Cy5.5, CD19-PE CF594, CD8a, Ter119, NK1.1-PE-Cy5, CD4-APC, CD49b-AF700, and Siglec F-APC-Cy7. For bone marrow samples the following antibodies were used: Live Dead Blue (1:2400), CD45-BUV 395, CD48-BUV 563 (1:100), CD41-BUV661 (1:100), CD117-BV421 (1:100), I-A/I-E-Pacific Blue (1:100), Ly6c-BV510, CD150-BV605 (1:100), Ly6g-BV650, CD16/32-BV711, CD11b-BV785, CD34-FITC (1:100), Fcerla-PerCP-Cy5.5 (1:100), CD135-PerCP-Fluor710 (1:100), CD125-PE (1:100), Sca-1-PE-Dazzle (1:100), CD3, NK1.1, B220, Ter 119, IL-7R-PE-Cy5, Integrin b7-AF647, and CD115-APC-Cy7 (1:100). ATMs (CD11b+, F4/80+, CD64+, CD9—, CD63−), LAMs (CD11b+, F4/80+, CD64+, CD9+, CD63+), monocytes (CD11b+, Ly6c+), and neutrophils (CD11b+, Ly6g+) were identified in eWAT of obese B6 mice as previously described,8 and sort-purified using an FACSAria (BD) or Aurora CS sorter (Cytek).

### Differentiation of 3T3-L1 Cells and Primary Preadipocytes

3T3-L1 cells were grown in tissue culture treated plates until cells reach 100% confluency, and then incubated additional 48 hr. Adipocyte differentiation was induced by induction media consisting of DMEM with 10% FBS, 500 μM IBMX, 1 μM dexamethasone, and 3 μg/ml insulin. After two days, induction media was replaced with adipocyte maintenance media consisting of DMEM with 10% FBS and 3 μg/ml insulin. Cells were grown in maintenance media until fully differentiated into lipid laden adipocytes (~10-15 days).

eWAT and iWAT were dissected, digested, and processed as described above. The SVF pellet was resuspended in media, filtered through 40 μM cell strainer, and centrifuged. SVF pellet was resuspended in preadipocyte expansion media consisting of DMEM/F12 with 10% FBS and 10 ng/ml Fgf2 and directly plated into 24-well plate. Cells were incubated an additional 48 hrs. after reaching 100% confluency and then differentiation was induced using media consisting of DMEM/F12 with 10% FBS, 500 μM IBMX, 1 μM rosiglitazone, 1 μM dexamethasone, and 3 μg/ml insulin. Two days post-induction of differentiation cells were grown in adipocyte maintenance media (DMEM/F12 with 10% FBS, 1 μM rosiglitazone, and 3 μg/ml insulin) until fully differentiated (7-10 days).

### Ex Vivo Bone Marrow-Derived Macrophage (BMDM) Differentiation and Polarization

Femurs from euthanized mice were dissected and flushed with cold PBS to collect bone marrow cells. Cells were filtered through 40 μM cell strainer and centrifuged. Cells were resuspended in BMDM differentiation media consisting of DMEM with 10 ng/ml M-CSF and plated into 10 cm plates and cultured until fully differentiated (6-7 days). Differentiated cells were stripped from 10 cm plate and centrifuged. Cell pellets were resuspended in M1 polarization media (DMEM with 10 ng/ml M-CSF, 5 ng/ml LPS and 15 ng/ml IFN-γ), M2 polarization media (DMEM with 10 ng/ml M-CSF and 15 ng/ml IL-4), or media (DMEM with 10 ng/ml M-CSF) supplemented with 300 μM of palmitate or arachidonate and plated into 12-well assay plates. RNA for qPCR was extracted from cells after 24 hrs. of polarization.

To quantify macrophage proliferation, 525,000 bone marrow cells were plated in a 24-well plate and live cells were quantified after 5 days of incubation in BMDM differentiation media.

Co-Culture of Primary Adipocytes with Extracellular Vesicles (EVs)

BMDMs from wild type (WT) and miR-6236 knockout (KO) mice were differentiated and metabolically activated with palmitate as described above. Cells were grown in metabolic activation media for 48 hours, and then exosomes were isolated from the cell culture media using total exosome isolation kit (Invitrogen, cat #4478359). Isolated exosomes were resuspended in PBS (1 ml PBS/10 ml culture media). Preadipocytes were isolated from stromal vascular fraction (SVF) of WT mice and differentiated into mature adipocytes in 24-well plate as described above. After full differentiation of adipocytes, old culture media was replaced with a new culture media supplemented with WT or KO exosomes (420 μl of media+80 μl exosome) and incubated for 24 hrs. Western blot and glucose uptake assays were performed after 24 hrs. of incubation.

### Purification of Extracellular Vesicles (EVs) for Transcriptomics and Treatments

For miRNA sequencing, LAMs were cultured overnight in 1 mL of serum-free media followed by isolation of the cellular fraction by ultracentrifugation and isolation of EVs from culture supernatant using ultracentrifugation (100,000×g for 2 hours, twice on the Optima L-90K Ultracentrifuge using a Beckman Coulter Ti-45 fixed angle rotor). Small RNAs were isolated and RNA-seq libraries generated using the Nucleospin miRNA isolation kit (Macherey-Nagel) and the SMARTer small RNA-seq kit (Clontech) following the manufacturer's instructions.

For in vitro treatments, BMDMs from WT or KO mice were differentiated and metabolically-activated with palmitate, as described above. Cells were grown in metabolic activation media with EV-depleted FBS (Neuromics) for 48 hours, and then EVs were purified from the cell culture media using total EV isolation kit (Invitrogen, 4478359).

For in vivo treatments, BMDMs from WT or KO mice were differentiated and metabolically-activated with palmitate, as described above. Cells were grown in metabolic activation media with EV-depleted FBS (Neuromics) for 48 hours, and then EVs were purified from the cell culture media by ultracentrifugation (100,000×g for 2 hours, twice on the Optima L-90K Ultracentrifuge using a Beckman Coulter Ti-45 fixed angle rotor).

EV Administration In Vivo and In Vitro Co-Culture with Primary Adipocytes

For in vivo treatment, isolated EVs were quantified by measuring the total EV protein content. Then, DIO male mice were given two doses of EVs 3 days apart by intraperitoneal injection (40 μg protein equivalent of EVs/mice/dose). Two days after the second dose, blood glucose and insulin level were measured, and mice were euthanized to harvest tissues for molecular assays.

For ex vivo co-culture, isolated EVs were resuspended in PBS (1 ml PBS/10 ml culture media). Preadipocytes were isolated from stromal vascular fraction (SVF) of WT mice and differentiated into mature adipocytes in a 24-well plates, as described above. After full differentiation of adipocytes, old culture media was replaced with a new culture media supplemented with WT or KO EVs (420 μl of media+80 μl EVs) and incubated for 24 hours. Western blot, glucose uptake, and lipolysis assays were performed after 24 hours of incubation.

TAT-Cre Treatment of BMDM for Functional Validation of loxP Sites in Fl+/+ Mice

BMDM were isolated and ex vivo differentiated as described above. After full differentiation, cells were stripped and plated in 24-well plate and incubated for 24 hrs. Cells were treated with either TAT-Cre (5 μM) or BSA and incubated an additional 24 hrs. Genomic DNA was extracted from cells and fragment size was analyzed after PCR amplification.

### In Vitro Glucose Uptake Assay

3T3-L1 cells were differentiated as described above and transfected with control siRNA, 30 nM miR-6236 mimic, 30 nM PTEN siRNA, or 30 nM each of miR-6236 mimic and PTEN SiRNA using Lipofectamine RNAiMAX transfection reagent (Invitrogen). Two days after transfection, culture media was removed and replaced with serum free media (DMEM with 2% BSA) and cell were incubated for ~3-5 hrs (basal glucose uptake). For insulin-stimulated glucose uptake, cells were insulin stimulated for ~30 min. in DMEM media with 10% FBS and 3 μg/ml insulin. Glucose uptake assay was performed using Glucose Uptake-Glo™ Assay kit (Promega).

### In Vitro Lipolysis Assay

3T3-L1 cells were differentiated, transfected, serum-starved, or insulin stimulated as above. For basal lipolysis, serum free media was replaced with fresh serum free media and cells were incubated for ~3-6 hrs. For insulin stimulated lipolysis, insulin media in the well was replaced with fresh insulin media and incubated for ~3-6 hrs. After incubation, media was collected, and glycerol content was measured using Free Glycerol Assay Kit (Abcam).

### In Vitro Lipogenesis

3T3-L1 cells were cultured and induced for differentiation as described above. 3-4 days post-induction, and just before appearance of visible lipid droplets, cells were transfected as described above. Three days after transfection, cells were stained with Oil Red O dye for total cellular lipids and imaged by EVOS FL Auto microscope (Thermo Scientific). Oil Red O dye from the stained cells was extracted using isopropanol and OD was measured at 515 nm wavelength.

### PTEN UTR Cloning and Dual Luciferase Reporter Assay

A segment of Pten 3′UTR (1,067 bp) harboring two miR-6236 predicted sites for mature sequences miR-6236e and miR-6236f was amplified from genomic DNA using primer pairs given in Table 1. A mutant Pten 3′UTR with an identical sequence other than deletion of the two miR-6236 predicted binding sites was synthesized by Life Technologies Corporation. Similarly, WT and mutant 3′UTRs for human Pten and murine Prkca were synthesized by Life Technologies Corporation. 3′ UTRs and pmirGLO plasmid vector (Promega) were separately restriction digested (with Nhei and Sbfl), purified, and the 3′UTR inserts were separately ligated into the plasmid just downstream of firefly luciferase gene open reading frame using T4 DNA ligase. Recombinant plasmid vectors were independently transfected into 293T cells along with control siRNA or miR-6236 mimic (miR-6236e or miR-6236f). The next day, firefly and renilla luciferase protein levels were measured using the Dual Luciferase Assay System (Promega) in a Biotek Synergy HTX plate reader with a dual injector (Agilent). Firefly luciferase signal was normalized to renilla luciferase signal to correct for differences in transfection efficiency among samples.

TABLE 1

| UTR cloning for miR-6236 | |
|---|---|
| target validation | |
| Pten 3′UTR_Forward | AGTCATGCTAGCTCTCAGTTGTAATGACTGCTCCAT |
| Pten 3′UTR_Reverse | TATGTTCCTGGAGGGTGTAAATCCTAAGAGCTTTGTTACA |
| qPCR primers | |
| Actb_F | CGCAGCCACTGTCGAGTC |
| Actb_R | GTCATCCATGGCGAACTGGT |
| CD206_F | TTCAGCTATTGGACGCGAGG |
| CD206_R | GAATCTGACACCCAGCGGAA |
| Arg1_F | GTACATTGGCTTGCGAGACG |
| Arg1_R | CGGCCTTTTCTTCCTTCCCAG |
| Stat1_F | GATCGCTTGCCCAACTCTTG |
| Stat1_R | ACTGTGACATCCTTGGGCTG |
| TNFa_Forward | GGTGCCTATGTCTCAGCCTC |
| TNFa_Reverse | GCTCCTCCACTTGGTGGTTT |
| CD9_Forward | TCTTCGGGTTCCTGTTGGTG |
| CD9_Reverse | GGGTTCATCCTTGCTCCGTA |
| Lipa_Forward | GCAAGTGGTCCGATTCCCTT |
| Lipa_Reverse | GGAGCAAAGCAGGCTCAGTA |
| Lpl_Forward | TCGTCATCGAGAGGATCCGA |
| Lpl_Reverse | TGTTTGTCGAGTGTCAGCCA |
| Plin2_Forward | TCCCTCAGCTCTCCTGTTAG |
| Plin2_Reverse | TGACATAAGCGGAGGACACA |
| Lipe_Forwar | AGAAGGATCGAAGAACCGCA |
| Lipe_Reverse | GTGTGAGAACGCTGAGGCTTT |

*SEQ ID NOs: 9-31 are shown in descending order

### Western Blots

Tissue samples and cultured cells were homogenized in T-PER™ tissue protein extraction reagent (Thermo Scientific) with Halt™ protease inhibitor cocktail using a Bead Rupture Elite homogenizer. Samples were separated on 4-12% Bis-tris precast mini gels and protein samples were transferred to PVDF membrane using Trans-Blot® Turbo™ transfer system (Bio-Rad). Primary antibodies used were anti-phospho AKT2 (Ser474) (Cell Signaling, 8599S; 1:5000 dilution), anti-AKT2 (Cell Signaling, 3063S, 1:5000 dilution), anti-PTEN (Santa Cruz Biotechnology, sc-7974, 1:1000 dilution), anti-p-HSL (Cell Signaling, 4126, 1:7000 dilution), anti-HSL (Cell Signaling, 4107, 1:7000 dilution) anti-actin (Sigma Aldrich, A2066-100UL, 1:5000 dilution), and anti-CD9 (BD Bioscience, 553758, 1:1000 dilution). Secondary antibodies used were anti-mouse IgG HRP (Cell Signaling, 7076S, 1:10,000 dilution), anti-rabbit IgG HRP (Novus Biologicals, HAF008, 1:5000 dilution), and anti-rat IgG HRP (Novus Biologics, 22109, 1:10,000). Protein bands on the membrane were detected by Enhanced chemiluminescent (ECL) substrate using a ChemiDoc MP (BioRad).

RNA Extraction and qPCR

Total RNA was extracted from frozen tissue or cultured cells using Trizol Reagent (Ambion, 15596018) following manufacturer's recommendation. cDNA was synthesized using Verso cDNA synthesis kit (Thermo Scientific, AB1453B) and qPCR reaction was carried out using PowerUp SYBR green master mix (Thermo Scientific, A25741) and a QuantStudio 12K Flex Real-Time PCR System (Applied Biosystem). miRNA qPCR was performed using the Taqman advanced miRNA assay kit (Thermo Fisher Scientific, A25576) following the manufacturer's instructions. Actin (Actb) and miR-21 were used as reference transcripts for normalization of mRNA and miRNAs, respectively. Primers used in qPCR are given in Table 1.

For tissue-level RNA-seq of eWAT and liver, libraries were prepared using the TruSeq RNA library prep kit (Illumina, RS-122-2001) and standard Illumina protocol. RNA-seq libraries were sequenced at single or paired-end, 75- to 100-bp read length on an Illumina HiSeq 2000.

### Purification of CD9+LAMs, LAM-Derived EVs, and Sequencing of Small RNAs

LAMs were identified in eWAT of obese B6 mice via their expression of CD64 and CD9 as previously described (Hill et al., 2018), and sort-purified using a FACSAria sorter (BD). LAMs were cultured overnight in 1 mL of serum-free media followed by isolation of the cellular fraction by centrifugation and isolation of EVs from culture supernatant using the Total Exosome Isolation kit (Invitrogen). Small RNAs were isolated and RNAseq libraries generated using the Nucleospin miRNA isolation kit (Macherey-Nagel) and the SMARTer smRNA-Seq kit (Clontech) following the manufacturer's instructions.

### Oil Red O Staining of Cultured Cells

Media was removed from the wells and cells were washed with PBS (1×) followed by fixing of cells with 4% paraformaldehyde for ~1 hr. Then, cells were washed (2×) with deionized water, incubated with working solution of Oil Red O for ~10 min, washed (4×) again with deionized water and then imaged. For quantification of total lipid Oil Red O from stained cells was extracted with 100% isopropanol and OD was measured at 515 nm.

### Epididymal and Inguinal White Adipose Tissue Histology

Adipose tissue was washed in PBS and fixed in 4% buffered paraformaldehyde solution overnight at 4° C. followed by ethanol dehydration. Dehydrated samples were sent to CHOP histology core to perform hematoxylin and eosin staining. Images were acquired using EVOS FL Auto microscope (Thermo Fisher Scientific) and adipocyte size was measured using Adiposoft plugin in the Fiji application.

### Hyperinsulinemic-Euglycemic Clamp

Hyperinsulinemic-euglycemic clamp studies were performed by the Penn Diabetes Research Center Rodent Metabolic Phenotyping Core (University of Pennsylvania); +/−or −/−littermate mice were bred in the Hill Lab and co-housed in mixed cages to minimize variability due to environmental factors. Hyperinsulinemic-euglycemic clamp studies were performed as previously described, 48,49 with some modifications. Indwelling jugular vein and carotid artery catheters were surgically implanted in the mice for infusion 7 days prior to the clamp study day. Mice were acclimated to the containers (plastic bowl with alpha dry) and fasted for 5 hours prior to initiation of clamp. Jugular vein and arterial line are hooked up to the dual swivel 2 hours prior to the clamp initiation. During the 90 min basal period, mice received a bolus infusion of 1.5 μCi [3-3H]-D-glucose followed by a constant infusion via the jugular vein at 0.075 μCi/minute. Baseline measurements were determined in blood samples collected at −10 and 0 min (relative to the start of the clamp) for analysis of glucose and [3-3H] glucose specific activity. The clamp was started at t=0 min with a primed-continuous infusion of human insulin (2.5 mU/Kg/min; Novolin Regular Insulin) and a donor blood infusion at 4.5 μL/min to prevent a 5% drop in the hematocrit. Blood glucose levels were clamped at euglycemia and tracer specific activity was maintained with a variable infusion of 50% dextrose+3-[3H]-D-glucose (0.05 μCi/μl in D50) for 2 hours. Blood samples were taken at t=80-120 min for the measurement of [3-3H] glucose specific activity and glucose infusion rates were adjusted to maintain euglycemia. 120 minutes after initiation of clamp, 12 mCi of 2-[14C] deoxyglucose ([14C] 2DG) was administered as a bolus and arterial blood samples were obtained at 2, 5, 10, 15 and 25 min to determine Rg, an index of tissue-specific glucose uptake in various tissues. After the final blood sample, animals were injected with a bolus of pentobarbital, and tissues were collected and frozen in liquid nitrogen and stored at −80° C. for subsequent analysis.

Processing of samples and calculations: Radioactivity of [3-3H] glucose, [14C] 2DG and [14C] 2DG-6-phosphate were determined, as previously described.⁴⁹ The glucose turnover rate (total Ra; mg/kg/min) was calculated as the rate of tracer infusion (dpm/min) divided by the corrected plasma glucose specific activity (dpm/mg) per kg body weight of the mouse. Tissue specific glucose disposal (Rg; mmol/100g tissue/min) was calculated, as previously described.⁴⁹

### Bioinformatics Analyses

During the initial discovery of mouse miR-6236 in LAM EVs, small RNA-seq reads from the eWAT LAM EVs were aligned to the mouse genome, mm10, using STAR aligner.⁵⁰ miRNA abundance was quantified using miRBase³⁸ annotation and FeatureCounts.⁵¹

Before analyzing any sequencing data, barcodes and sequencing adapters in the library were removed and reads were filtered based on the quality score. All the analyses except assembly of human transcripts from Drosha KO cell lines were performed in CLC Genomics Workbench Suite (Qiagen). Human pri-miRNA transcripts in Drosha KO cell lines were assembled using HISAT2 sequence aligner (Kim et al., 2019) and StringTie transcript assembler (Pertea et al., 2015) in Cyverse Discovery Environment Suite. For mice miRNA expression analysis, miRNA sequences in miRbase release 22.1 were used as a reference. For human miRNAs expression analyses, newly discovered has-miR-6236 was also added to the miRbase reference before using it as a reference. Read tracks from small RNA-Seq, argonaute HITS-CLIP-Seq and ATAC-Seq datasets were generated by mapping the reads to respective human or mice genome in CLC genomics, and read tracks were visualized by Integrated Genome Browser (IGB). Differentially-expressed genes between tissue conditions were identified as FC >2, FDR <0.05, >1 RPKM.

### In-Lab Generated, and Public Sequencing Data Used in the Study

In-lab generated raw small RNA sequencing data from CD9+ATM EVs fractions are deposited in Mendeley under accession number [doi: 10.17632/cbszxmd63x.1]. NCBI locations of raw public data used in this study are as follows: murine tissue and ATM derived EV small RNA-seq (GEO: GSE119661, GSE142677, GSE97652); murine adipose tissue argonaute HITS-CLIP (GEO: GSE142677); murine Kupffer cell small RNA-seq (GEO: GSE160016); young (6 month old) and aged (24 month old) murine serum small RNA-Seq (GEO: GSE76442); murine peritoneal macrophage PPARγ ChIP-seq (GEO: GSM3022232); murine LAM ATAC-seq (GEO: GSE113583); 3T3-L1 and primary adipocyte small RNA-Seq (Bio project #PRJEB20090, PRJEB25978); Human Drosha KO RNA-seq (PRJNA282167); Human serum small RNA-seq (PRJEB21747); Human adipose tissue small RNA-seq (GSE45159); Human argonaute HITS-CLIP (GSE41272, GSE41357) and human adipocytes ATAC-seq (GSE178796).

The following examples are provided to illustrate certain embodiments of the invention. They are not intended to limit the invention in any way.

### Example I

### miR-6236 is a LAM-Secreted miRNA that is Enriched in Adipose Tissue and Altered During Obesity

Given the beneficial functions of LAMs during obesity, and their ability to secrete EVs, we sought to investigate whether LAM-derived EVs contain metabolically relevant miRNAs. To do so, we performed small RNA sequencing on EVs purified from ex vivo LAM and characterized the genomic and molecular characteristics of a novel, LAM-secreted miRNA (miR-6236). We studied the cellular and organism-level functions of miR-6236 using novel loss-of-function mouse strains complemented by in vitro gain-of-function approaches. We identified Pten, a known negative regulator of insulin signaling, as a miR-6236 target. Finally, we performed analyses of human transcriptome and epigenome sequencing data to identify a homolog of miR-6236 and perform correlation studies with several key obesity-associated clinical outcomes. By doing so, we have characterized a novel mechanism by which ATMs function and identified a molecule that may be of therapeutic relevance to the treatment of T2DM.

### miR-6236 is a LAM-Secreted miRNA that is Enriched in Adipose Tissue and Altered During Obesity

To characterize the LAM miRNA-secretome, small RNA sequencing on purified EV fractions of ex vivo LAM cultures was performed. miR-6236 was identified as the most abundant miRNA in LAM-derived EVs (FIGS. 1A, and 1B). We also performed small RNA-sequencing on EVs purified from ex vivo LAM cultures isolated from wild-type (WT) mice subjected to a model of diet-induced obesity (DIO) (FIG. 1G). We found that miR-6236 is the most abundant miRNA in LAM-derived EVs (FIG. 1A-1B)

While miR-6236 has been predicted computationally, its sequence, structure, and functions have not been previously validated. To validate miR-6236, we combined our sequencing data with 324 previously published small RNA-Seq libraries encompassing 19 tissues, ATMs, and ATM-derived exosomes (Kern et al., 2020; O'Connor et al., 2021; Ying et al., 2017), and aligned reads from these libraries to the pre-miR-6236 locus. The majority of RNA-Seq reads aligned to a region upstream of the predicted mature miR-6236 sequence (FIG. 1C). We next used legacy data to map adipose tissue Argonaute high-throughput sequencing of RNA isolated by crosslinking immunoprecipitation (HITS-CLIP-Seq) reads to the pre-miR-6236 locus (O'Connor et al., 2021). Consistent with our RNA-seq alignment, HITS-CLIP reads predominantly mapped to a region upstream of the predicted mature miR-6236 sequence (FIG. 1C). Together, these data indicate that prior miR-6236 annotations are inaccurate and provide more accurate resolution of the miR-6236 locus.

We next reannotated the pre-miR-6236 sequence and found that pre-miR-6236 has more than one predicted stem and loop, and several mature sequence isoforms (FIG. 1D, Table 2). To determine the expression pattern of miR-6236, we reanalyzed tissue small RNA sequencing data from prior studies (Kern et al., 2020; O'Connor et al., 2021). This analysis revealed that, of the 16 tissues analyzed, miR-6236 expression was largely restricted to adipose tissue and bone marrow (FIG. 1E). A similar expression pattern was observed in DIO WT mice (FIG. 1E). To validate our findings, and determine if miR-6236 expression is dynamically regulated by obesity, we analyzed small RNA sequencing data from ATM-derived EVs (Ying et al., 2017). Consistent with our own data, we found that miR-6236 is elevated in ATM derived EVs of obese mice compared to those of lean mice (FIG. 1F). Together, these findings indicate miR-6236 is a novel, LAM-secreted miRNA that is enriched in adipose tissue and positively regulated by obesity.

TABLE 2
Mature Sequence Isoforms of miR-6236 in adipose tissue

| | | | | | |
|---|---|---|---|---|---|
| | | | Read | Read per | SEQ ID |
| Custom ID | Mature sequence | Length | count | million (RPM) | NO: |

Murine Sequence Isoforms

| | | | | | |
|---|---|---|---|---|---|
| miR- | CACCTGCCGAATCAACTAGC | 20 | 4288 | 79.25 | 32 |
| 6236a | | | | | |
| miR- | ACCTGCCGAATCAACTAGC | 19 | 3050 | 56.37 | 33 |
| 6236b | | | | | |
| miR-6236c | TCACCTGCCGAATCAACTAGC | 21 | 2797 | 51.69 | 34 |
| miR- | ACTCACCTGCCGAATCAACTAGC | 23 | 2630 | 48.61 | 35 |
| 6236d | | | | | |
| miR- | CCTGAAAATGGATGGCGCTGGAGC | 24 | 2413 | 44.60 | 36 |
| 6236e | | | | | |
| miR-6236f | CCGAATCAACTAGCCCTGA | 19 | 2362 | 43.65 | 37 |
| miR- | CGAATCAACTAGCCCTGAAA | 20 | 2305 | 42.60 | 38 |
| 6236g | | | | | |
| miR- | CTCACCTGCCGAATCAACTAGC | 22 | 2180 | 40.29 | 39 |
| 6236h | | | | | |
| miR-6236i | AACTCACCTGCCGAATCAACTAGC | 24 | 2108 | 38.96 | 40 |
| miR-6236j | CAACTCACCTGCCGAATCAACTAGC | 25 | 2083 | 38.50 | 41 |
| miR- | CCCTGAAAATGGATGGCGCTG | 21 | 2026 | 37.44 | 42 |
| 6236k | | | | | |
| miR-6236l | CTGAAAATGGATGGCGCTGGAGC | 23 | 1761 | 32.55 | 43 |
| miR- | GAAAATGGATGGCGCTGGAGC | 21 | 1632 | 30.16 | 44 |
| 6236m | | | | | |
| miR- | TGCCGAATCAACTAGCCCTGAAA | 23 | 1557 | 28.78 | 45 |
| 6236n | | | | | |
| miR- | GAATCAACTAGCCCTGAAA | 19 | 1503 | 27.78 | 46 |
| 6236o | | | | | |
| miR- | TGAAAATGGATGGCGCTGGAGC | 22 | 1372 | 25.36 | 47 |
| 6236p | | | | | |
| miR- | AAAATGGATGGCGCTGGAGC | 20 | 1247 | 23.05 | 48 |
| 6236q | | | | | |
| miR-6236r | CTGCCGAATCAACTAGCCCT | 20 | 1238 | 22.88 | 49 |
| miR-6236s | AAATGGATGGCGCTGGAGC | 19 | 1145 | 21.16 | 50 |
| miR-6236t | GCCGAATCAACTAGCCCTGA | 20 | 1043 | 19.28 | 51 |
| miR- | ACAACTCACCTGCCGAATCAAC | 22 | 1036 | 19.15 | 52 |
| 6236u | | | | | |
| miR- | CCTGCCGAATCAACTAGCCCT | 21 | 586 | 10.83 | 53 |
| 6236v | | | | | |
| miR- | AGCCCTGAAAATGGATGGCGCTGGAGC | 27 | 446 | 8.24 | 54 |
| 6236w | | | | | |
| miR-6236x | TAGCCCTGAAAATGGATGGCGCTGGAGC | 28 | 410 | 7.58 | 55 |
| miR- | AATCAACTAGCCCTGAAAA | 19 | 396 | 7.32 | 56 |
| 6236y | | | | | |
| miR-6236z | GCCCTGAAAATGGATGGCGCTGGAGC | 26 | 359 | 6.63 | 57 |

Human Sequence Isoforms

| | | | | | |
|---|---|---|---|---|---|
| Hu-miR- | AAAATGGATGGCGCTGGAGC | 20 | 2831 | — | 58 |
| 6236-1 | | | | | |
| Hu-miR- | AAATGGATGGCGCTGGAGCG | 20 | 4176 | — | 59 |
| 6236-2 | | | | | |
| Hu-miR- | AACTAGCCCTGAAAATGGATG | 21 | 7177 | — | 60 |
| 6236-3 | | | | | |
| Hu-miR- | AAGGAGTGTGTAACAACTCACC | 22 | 5657 | — | 61 |
| 6236-4 | | | | | |
| Hu-miR- | AATCAACTAGCCCTGAAAATGG | 22 | 5713 | — | 62 |
| 6236-5 | | | | | |
| Hu-miR- | AATGGATGGCGCTGGAGCG | 19 | 2864 | — | 63 |
| 6236-6 | | | | | |
| Hu-miR- | ACTAGCCCTGAAAATGGATG | 20 | 1473 | — | 64 |
| 6236-7 | | | | | |
| Hu-miR- | AGGAGTGTGTAACAACTCACC | 21 | 3606 | — | 65 |
| 6236-8 | | | | | |
| Hu-miR- | ATCAACTAGCCCTGAAAATGG | 21 | 3484 | — | 66 |
| 6236-9 | | | | | |
| Hu-miR- | CACCTGCCGAATCAACTAGCCC | 22 | 8764 | — | 67 |
| 6236-10 | | | | | |
| Hu-miR- | CCCTGAAAATGGATGGCGCTG | 21 | 1798 | — | 68 |
| 6236-11 | | | | | |
| Hu-miR- | CCGGCAGTCGAGAGTGGACGG | 21 | 5472 | — | 69 |
| 6236-12 | | | | | |
| Hu-miR- | CCTGAAAATGGATGGCGCTG | 20 | 1296 | — | 70 |
| 6236-13 | | | | | |
| Hu-miR- | CCTGCCGAATCAACTAGCCCTG | 22 | 1280 | — | 71 |
| 6236-14 | | | | | |
| Hu-miR- | CGAATCAACTAGCCCTGAAAATGG | 24 | 2915 | — | 72 |
| 6236-15 | | | | | |
| Hu-miR- | CGCTAAGGAGTGTGTAACAACTC | 23 | 2292 | — | 73 |
| 6236-16 | | | | | |
| Hu-miR- | CGGCAGTCGAGAGTGGACGG | 20 | 5923 | — | 74 |
| 6236-17 | | | | | |
| Hu-miR- | CTAAGGAGTGTGTAACAACTCACC | 24 | 2605 | — | 75 |
| 6236-18 | | | | | |
| Hu-miR- | CTAGCCCTGAAAATGGATG | 19 | 1440 | — | 76 |
| 6236-19 | | | | | |
| Hu-miR- | CTCACCTGCCGAATCAACTAGCCCT | 25 | 1171 | — | 77 |
| 6236-20 | | | | | |
| Hu-miR- | CTGAAAATGGATGGCGCTGG | 20 | 3222 | — | 78 |
| 6236-21 | | | | | |
| Hu-miR- | GAAAATGGATGGCGCTGGAGC | 21 | 3919 | — | 79 |
| 6236-22 | | | | | |
| Hu-miR- | GAATCAACTAGCCCTGAAAATGG | 23 | 9374 | — | 80 |
| 6236-23 | | | | | |
| Hu-miR- | GCCGGCAGTCGAGAGTGGACGG | 22 | 6062 | — | 81 |
| 6236-24 | | | | | |
| Hu-miR- | GCTAAGGAGTGTGTAACAACTC | 22 | 9486 | — | 82 |
| 6236-25 | | | | | |
| Hu-miR- | GGCAGTCGAGAGTGGACGG | 19 | 4760 | — | 83 |
| 6236-26 | | | | | |
| Hu-miR- | TCACCTGCCGAATCAACTAGCCCT | 24 | 4664 | — | 84 |
| 6236-27 | | | | | |
| Hu-miR- | TGAAAATGGATGGCGCTGGAGC | 22 | 6545 | — | 85 |
| 6236-28 | | | | | |

As miR-6236 is a novel miRNA, we further characterized its expression pattern across various cell types and under different physiological conditions using newly-generated and publicly available data sets. First, we examined expression of miR-6236 in sort-purified epidydimal white adipose tissue (eWAT) myeloid immune cells of DIO WT mice. We observed that miR-6236 was present in macrophages, monocytes, and neutrophils, but was expressed at a significantly lower level in neutrophils compared with macrophages and monocytes (FIG. 1H). To determine whether miR-6236 was secreted from macrophages and monocytes, we compared miR-6236 levels in EVs purified from cultures of DIO WT eWAT Ly6c+ monocytes, CD9-10 ATMs, and CD9+LAMs.⁸ We observed that miR-6236 was present in EVs derived from all myeloid subsets, but was enriched in EVs derived from ATMs and LAMs relative to monocytes (FIG. 1I). As miR-6236 expression was enriched in EVs derived from ATMs and LAMs, we next examined the relative capacity for these two macrophage populations to secrete EVs. We found that LAMs secreted nearly twice as many EVs per cell as compared with CD9-ATMs (FIG. 1J). As LAMs are characterized by CD9 expression, we also confirmed that LAM-derived EVs contain CD9 (FIG. 1K).

To determine if miR-6236 was expressed by other metabolic tissue-resident macrophages, we analyzed Kupffer cell small RNA-seq data from DIO WT and Trem2 knockout mice.¹⁷ We detected miR-6236 in Kupffer cells (FIG. 1L). Notably, miR-6236 expression did not require the presence the of Trem2 in Kupffer cells.⁹ Next, we examined the effect of age on miR-6236 expression by analyzing small RNA-seq data from the serum of young (6 months) or aged (24 months) mice.¹⁸ Consistent with secretion in EVs, miR-6236 was detected in the serum of these mice, but its level was not significantly different between young and aged mice (FIG. 1M). Finally, to determine potential upstream regulators of miR-6236 expression, we treated BMDMs with various cytokines and fatty acids and measured miR-6236 expression by qPCR. Fatty acids palmitate and arachidonate upregulated, while M1 (LPS+IFNγ) and M2 (IL-4) polarizing agents downregulated, the expression of miR-6236 (FIG. 1N).

To examine potential epigenetic mechanisms of regulation of the pre-miR-6236 locus, we utilized legacy data to map macrophage PPARg chromatin immunoprecipitation with sequencing (ChIP-seq) and LAM assay for transposase-accessible chromatin with sequencing (ATAC-seq) reads to the pre-miR-6236 locus.^{8,19} We found overlapping ATAC-seq and PPARg ChIP-seq peaks at the pre-miR-6236 locus (FIG. 10) indicating a means of transcriptional regulation. Together, these data indicate that miR-6236 is a novel, myeloid-secreted miRNA that is enriched in adipose tissue and positively regulated by obesity and metabolic macrophage activation.

miR-6236 Protects Against the Development of Obesity-Associated Metabolic Outcomes

To investigate the in vivo functions of miR-6236, we generated a whole-body miR-6236 knockout (KO) mouse line by deleting the DNA sequence encoding entire pre-miR-6236 locus in the C57BL/6 background (FIG. 2A), and evaluated obesity-associated outcomes by subjecting KO and WT littermate control mice to our DIO model. The CRISPR-targeted miR-6236 locus was entirely deleted in KO mice and confirmed by DNA gel genotyping and miR-6236 qPCR of BMDM-derived EVs (FIGS. 2B and 2T).

We next evaluated obesity-associated outcomes by subjecting KO or wild-type littermate control (WT) mice to an established model of diet-induced obesity (DIO) (Hill et al., 2018). Body weight did not differ between lean WT or KO mice (FIG. 2C), nor did body weight differ between obese WT or KO mice (FIG. 2D and FIG. 2E). However, obese KO male mice displayed elevated ad libitum fed and fasting serum glucose levels, elevated ad libitum fed serum insulin levels, and worse fasting glucose tolerance compared to obese WT male mice (FIGS. 2F-2H, 2W). DIO KO male mice also had elevated serum free fatty acid and glycerol levels compared with DIO WT male mice (FIG. 2K, 2L). A similar phenotype was observed in obese female KO mice (FIGS. 21-2J and 2U-2V). Obese KO male mice also displayed elevated serum free fatty acid and glycerol levels compared with obese WT male mice (FIGS. 2K, 2L), while serum glucagon, triglyceride, and cholesterol levels did not differ between obese WT and KO mice (FIGS. 2L-20). Together, these observations indicate that miR-6236 protects against the development of obesity-associated adverse metabolic outcomes.

To further interrogate the metabolic alterations observed in KO mice, we used a hyperinsulinemic-euglycemic clamp to measure insulin sensitivity. These studies were performed with +/−or −/− littermates that were co-housed in mixed cages, to minimize variability due to environmental factors. Briefly, mice received a constant infusion of insulin and radiolabeled glucose was co-infused, as needed, to maintain euglycemia. To achieve euglycemia, DIO KO mice required a lower glucose infusion rate (FIGS. 2P and 2X) and had a trend towards lower peripheral glucose disposal (FIG. 2Q), as compared with DIO littermate controls. Further, measurement of radiolabeled glucose accumulation revealed a trend towards a defect in glucose uptake by eWAT, but not other adipose tissue depots, of DIO KO mice as compared to DIO littermate controls (FIG. 2W). We also observed a trend towards a defect in glucose uptake by vastus lateralis, but not other muscle depots, of DIO KO mice as compared to DIO littermate controls (FIG. 2S). We did not observe a difference in basal or insulin-mediated suppression of hepatic glucose production between DIO KO and DIO littermate control mice (FIG. 2Y). Together, these data indicate that miR-6236 promotes insulin sensitivity and protects against the development of adverse metabolic outcomes during obesity.

miR-6236 Promotes Insulin-Mediated Signaling and Functions in Adipocytes.

In adipocytes, insulin has multiple functions that maintain metabolic homeostasis including promoting glucose uptake and lipogenesis, and inhibiting lipolysis (Santoro et al., 2021). Given the global metabolic changes observed in KO mice, and the high degree of expression of miR-6236 in adipose tissue, we hypothesized that miR-6236 was influencing adipocyte insulin signaling during obesity. To test this, we examined the cellular, molecular, and metabolic phenotype of key metabolic tissues in obese WT or KO mice. The tissue weight of epidydimal white adipose tissue (eWAT) and liver were not different between obese WT or KO mice (FIG. 3A), though livers of DIO KO mice had higher triglyceride content as compared with those of DIO WT mice (FIG. 3Q). Further, both liver and eWAT of DIO KO mice had non-significant decreases in the expression of de novo lipogenesis genes as compared with those of DIO WT mice (FIG. 3R). Upon examination of adipose tissue histology, we observed that eWAT adipocyte size was slightly larger in DIO KO as compared with DIO WT mice (FIG. 3S), while iWAT adipocyte size was unchanged (FIG. 3T).

Expression of the insulin receptor (INSR) in eWAT did not differ between DIO WT and DIO KO mice (FIG. 3B). However, while expression of the insulin receptor (IsnR) was not significantly different between obese WT and KO mice (FIG. 3B), phosphorylation of Akt serine/threonine kinase 2 (pAkt2) was significantly lower in obese eWAT of KO as compared with WT mice (FIG. 3C). To better understand the functional consequences of this defect, we examined measures of insulin signaling in adipocytes including glucose uptake and lipolysis. Ex vivo insulin-stimulated glucose uptake was lower in adipocytes isolated from eWAT of obese KO as compared with WT mice (FIG. 3D), while lipolysis was increased (FIG. 3E). Consistently, expression of genes central to lipolysis were elevated in eWAT of obese KO as compared with WT mice (FIG. 3F), as were levels of phosphorylated hormone sensitive lipase (pHsl) (FIG. 3G). Notably, pAKT2 phosphorylation was not altered in eWAT of lean KO mice as compared to WT mice (FIG. 3U), an observation consistent with observed blood glucose and insulin measurements. In sum, these findings are consistent with reduced adipocyte insulin signaling in DIO KO as compared with DIO WT mice.

To further establish the effects of miR-6236 on adipocyte insulin-dependent functions, we performed gain of function studies by transfecting miR-6236 mimic or control miRNA into in vitro differentiated 3T3-L1 adipocytes. Transfection with miR-6236 mimic increased 3T3-L1 adipocyte Akt phosphorylation, insulin-stimulated glucose uptake, and insulin-mediated suppression of lipolysis (FIGS. 3H-3J). Finally, to determine whether macrophage-derived EVs were sufficient to mediate the effects of miR-6236 on adipocyte insulin-dependent functions, we metabolically activated bone marrow-derived macrophages from KO or WT mice, purified BMDM-secreted EVs, and treated differentiated WT primary adipocytes with EV fractions. Treatment of primary adipocytes with EVs isolated from WT BMDMs resulted in increased Akt phosphorylation and insulin-stimulated glucose uptake as compared with EVs isolated from KO BMDMs (FIGS. 3K-3M).

Finally, to investigate the effects of EV-delivered miR-6236 in vivo, we intraperitoneally (i.p.) injected WT or KO BMDM-derived EVs into DIO WT mice and measured insulin-dependent phenotypes. Mice that received WT EVs had reduced blood glucose and serum insulin levels, and increased eWAT AKT2 phosphorylation, as compared to mice that received KO EVs (FIGS. 3N-3P). Together, these results indicate that macrophage EV-derived miR-6236 is sufficient to promote insulin-mediated signaling and related functions in adipocytes and adipose tissue.

miR-6236 Augments Insulin Signaling in Adipocytes by Inhibiting PTEN

We next sought to determine a mechanism by which miR-6236 regulates insulin signaling in adipocytes. miRNAs bind to the 3′UTR of target mRNAs leading to suppression of translation or mRNA degradation (Eulalio et al., 2008). To identify putative miR-6236 targets, we performed computational assessment of predicted miR-6236 binding sites in 3′UTRs of mRNA transcripts genome-wide using sRNAtoolbox package (Aparicio-Puerta et al., 2019). We then focused our validation efforts on targets known to negatively regulate insulin signaling (FIG. 4A). One such potential target was Pten, the mRNA product of which contains at least 7 predicted miR-6236 binding sites in its 3′UTR. PTEN antagonizes PI3K by dephosphorylating phosphatidylinositol-3,4,5-triphosphate thereby inhibiting insulin signaling (Maehama and Dixon, 1998; Nakashima et al., 2000). Various knockout animal studies have shown that interfering with PTEN action improves insulin-dependent cellular metabolism and global insulin sensitivity and glucose homeostasis (Li et al., 2020). As such, Pten was deemed to have strong potential to be regulated by miR-6236 and contribute to the phenotype of miR-6236 KO mice.

To determine if PTEN is post-transcriptionally regulated by miR-6236, we first sought to determine if miR-6236 binds to the 3′UTR of the Pten mRNA. To do so, we cloned a ~ 1 kb region of the Pten 3′UTR harboring 2 of the 7 predicted miR-6236 binding sites downstream of the firefly luciferase gene open reading frame (WT 3′UTR) (FIGS. 4B, 4C). We also generated a second construct that lacked the two predicted binding sites in the Pten 3′UTR (Mutant 3′UTR). We then tested the effects of miR-6236 or a control miRNA on luciferase expression, in vitro. We found that miR-6236 mimic reduced firefly luciferase expression from the WT Pten construct, but not from the Mut Pten construct, indicating that miR-6236 binds to the predicted target sites in the 3′UTR of the Pten mRNA and interferes with translation (FIG. 4D).

We next sought to determine if mimicking the function of miR-6236 influences PTEN protein levels and insulin signaling in adipocytes. To do so, we transfected differentiated 3T3-L1 adipocytes with control miRNA or miR-6236 mimic molecules and measured PTEN protein levels. Upon transfection of 3T3-L1 adipocytes with miR-6236 mimic we observed a reduction in PTEN protein levels (FIG. 4E), with an appropriate compensatory increase in Akt phosphorylation (FIG. 4F). Effects of miR-6236 were similar to those observed with a small interfering RNA targeting PTEN (PTEN siRNA), and there was no additive effect of miR-6236 and PTEN siRNA (FIGS. 4E and 4F), suggesting that miR-6236 and PTEN siRNA are acting through the same mechanism to influence Akt phosphorylation. Furthermore, miR-6236 and PTEN siRNA transfection resulted in similar increases in 3T3-L1 adipocyte glucose uptake and lipid accumulation (FIGS. 4G, 4H), further supporting that miR-6236 acts through PTEN to mediate its effects on adipocyte biology. Finally, we found that PTEN protein levels are elevated in eWAT of miR-6236 KO as compared with WT mice (FIG. 4I). In contrast, PTEN protein levels in eWAT were reduced in DIO WT mice that received i.p. injections of WT EVs compared to mice that received miR-6236 deficient EVs (FIG. 4J). Together, these data validate PTEN as being post-transcriptionally regulated by miR-6236 in a manner that can augment insulin signaling in adipocytes and adipose tissue.

As miR-6236 is predicted to bind multiple targets (FIG. 4A),24 the function of miR-6236 may not be mediated solely via suppression of PTEN. As such, we also experimentally validated another top-predicted target of miR-6236, Prkca. We observed that miR-6236 binds to the 3′UTR of Prkca and suppresses subsequent gene translation (FIG. 4K). This indicates that miR-6236 regulates other molecules relevant to adipocyte insulin signaling during obesity.

### miR-6236 Does not have Major Effects on Immune Cell Populations in Peripheral Compartments

Given the high expression of miR-6236 in macrophages and bone marrow, we investigated how loss of miR-6236 affects the frequency and distribution of various immune cells in major immunological organs including the bone marrow (BM), blood, and spleen. The gating strategy used to profile different immune cell progenitors and mature myeloid and lymphoid lineages is provided in FIGS. 10 and 11. The number of CD45+ cells was reduced in BM of DIO KO compared to DIO WT mice (FIG. 12A). Among immune cell progenitors, the frequency and number of common myeloid progenitors (CMP) were slightly reduced in KO mice, while the frequency and number of the other immune cell progenitors were unchanged (FIG. 12B). Among mature bone marrow immune cells, the frequency and number of monocytes and polymorphonuclear leukocytes (PMNs) were reduced in KOs, though monocyte frequency as a percentage of total CD45+ cells was not significantly different between WT and KO mice (FIG. 12C). In the blood and spleen, neither the total number of CD45+ cells nor the percentage or number of any specific mature immune cell population was altered between DIO WT and DIO KO mice (FIGS. 12D-12G). Together, these data indicate that miR-6236 has minimal effects on the development and distribution of immune cells in peripheral organs.

### Myeloid Cell-Derived miR-6236 Influences Adipose Tissue Insulin Signaling and Global Metabolic Homeostasis During Obesity

As miR-6236 is secreted by LAMs, we hypothesized that LAM derived miR-6236 plays a cell-extrinsic role in regulating adipocyte insulin signaling during obesity. However, it is possible that miR-6236 has cell-intrinsic functions in ATMs or adipocytes that influence adipocyte insulin signaling (Hill et al., 2018; Jaitin et al., 2019). To test for macrophage-intrinsic functions, we profiled eWAT immune cell populations in obese WT or KO mice. We observed that adaptive and innate immune cell populations, including LAMs, are present in similar numbers in obese WT and KO mice (FIGS. 10A, 10B, and 5A). However, LAMs of KO mice display higher levels of intracellular lipid (FIG. 5B), consistent with their known functions related to lipid phagocytosis and the increased lipolysis observed in KO mice (Hill et al., 2018; Russo and Lumeng, 2018).

To test if miR-6236 influences macrophage activation or functional states, we next carried out ex vivo M1, M2, or metabolic activation of WT or KO BMDMs. Expression levels of key M1 (Stat1 and Tnfa), M2 (Arg1 and cd206), and metabolic activation (cd9 and Lipa) transcriptional markers were unaltered between appropriately polarized WT or KO BMDMs (FIGS. 5C-5E). Given that PTEN is a known tumor suppressor, we also investigated if miR-6236 affects cell proliferation. Upon culture of BMDMs ex vivo, we did not observe a difference in the capacity for cell proliferation between WT and KO BMDMs (FIG. 5K). To test if miR-6236 influences ATM polarization in vivo, we next investigated expression of select macrophage-related genes in sort-purified ATMs or LAMs from DIO WT or DIO KO mice. We observed some significant changes in expression of key ATM-related genes (Trem2, Lipa, Tnf, Tlr2, and Arg1) between DIO WT and DIO KO adipose macrophages (FIG. 5L, 5M). Together, the above studies indicate that miR-6236 influences ATM lipid accumulation and gene expression in vivo during obesity.

To complement the above studies of macrophages, we also sought to examine whether cell-intrinsic miR-6236 influences insulin-dependent adipocyte functions. First, we examined miR-6236 expression in primary and 3T3-L1 adipocytes. Adipocytes expressed low levels of miR-6236 (FIG. 5N). Next, we examined the effects of miR-6236 on adipocyte differentiation and function by differentiating preadipocytes from WT and KO mice into adipocytes ex vivo and measuring insulin-specific functions. We observed that adipocyte differentiation, PTEN expression, AKT phosphorylation, insulin-stimulated glucose uptake, and insulin-stimulated suppression of lipolysis were similar between ex vivo differentiated adipocytes from WT or KO mice (FIG. 5F-5J). The above observations indicate that miR-6236 is expressed at a low level in adipocytes, and that cell-intrinsic miR-6236 does not influence the development or tested insulin-dependent functions of adipocytes.

To formally establish whether myeloid-derived miR-6236 is responsible for the phenotype observed in KO mice, we generated miR-6236/1/fl mice using the CRISPR-Cas9 system. To do so, LoxP sequences were inserted flanking the endogenous miR-6236 locus (FIG. 6A). DNA gel genotyping (FIG. 6B), and in vitro treatment of BMDMs with TAT-CRE recombinase (FIG. 6C), were consistent with the functional insertion of flanking LoxP sequences. miR-6236^{fl/fl} mice were bred to mice expressing Cre recombinase under the control of the endogenous Lyz2 promoter (LysMCre) to generate mice in which immune cells of the myeloid lineage were deficient in miR-6236 (MKO). miR-6236 was absent from BMDM EVs derived from MKO mice (FIG. 6H), establishing the functionality of this mouse model.

Like whole-body KO mice, myeloid-specific deficiency in miR-6236 did not influence weight gain in either male or female mice (FIGS. 6D, 6E). However, both obese male and obese female MKO mice displayed elevated serum glucose and insulin levels when fed ad libitum compared with obese, sex-matched and Cre-littermate controls (FIGS. 7A, 7B and FIGS. 6F, 6G). Obese MKO mice also had worse fasting glucose tolerance and elevated serum free fatty acids when fed ad libitum compared with obese, sex-matched and Cre-littermate controls (FIGS. 7C, 7D). Finally, eWAT of obese MKO mice had a higher level of PTEN (FIG. 7E), and reduced level of phosphorylated AKT2 (FIG. 7F), compared with obese, sex-matched and Cre-littermate controls. Together, these data indicate that myeloid cell-derived miR-6236 influences adipose tissue insulin signaling and global metabolic homeostasis during obesity.

### a Human miR-6236 Homolog is Inversely Correlated with Obesity-Associated Outcomes

We next sought to determine if a miR-6236 homolog is present in the human genome. Discovering the novel miRNA gene solely based on mature sequence alignment or small RNA sequencing datasets is unreliable as short RNA reads can match nonspecifically to multiple genomic locations and reads generated from degraded RNA fragments may be mistaken as true mature miRNAs. Since miRNAs are first transcribed in the form of much longer primary miRNA transcript (pri-miRNA) before being cleaved into pre-miRNA (by Drosha) and mature miRNA (by Dicer), we first attempted to identify a homologous pri-miRNA transcript. As pri-miRNAs are transient and rapidly processed by Drosha, they are difficult to detect by conventional RNA sequencing in normal cells. To overcome this limitation, we assembled human RNA transcripts in a published deep RNA-Seq dataset from conditional Drosha knockout cells that is enriched for pri-miRNA transcripts (Chang et al., 2015). We discovered a novel, 135 nucleotide long transcript that mapped to an unannotated chromosome location (GRCH38 Chr4: 69431035-69430901, ‘-’ strand), hereafter referred to as pri-hsa-miR-6236. The pri-hsa-miR-6236 and pre-mmu-miR-6236 have a sequence alignment of 90 nucleotides with ~88% (79/90) sequence identity (FIG. 8A). Pri-hsa-miR-6236 has a predicted miRNA-like secondary structure in the aligned region that we designated as pre-hsa-miR-6236 (Genome coordinate: GRCH38 Chr4: 69430994-69430905; ‘-’strand) (FIG. 8B).

We next mapped small RNA-Seq and argonaute HITS-CLIP-Seq reads from published studies to pre-hsa-miR-6236 (Balakrishnan et al., 2014; Civelek et al., 2013; Haecker et al., 2012). We detected abundant mature miRNA fragments that mapped to the pre-hsa-miR-6236 transcript (FIG. 9A). Reanalysis of previously published adipocyte ATAC-Seq dataset showed presence of open chromatin along the pre-hsa-miR-6236 locus (FIG. 9A) (Perrin et al., 2021), further demonstrating a feature of a transcriptionally active gene. Finally, we reanalyzed previously published human serum and subcutaneous adipose tissue small RNA-seq datasets from lean and obese male subjects to determine whether obesity influenced hsa-miR-6236 expression (Civelek et al., 2013). We found that hsa-miR-6236 expression is elevated in obese subjects in both tissue types (FIGS. 9B, 9C). In obese subjects, hsa-miR-6236 level in serum is elevated to the extent that it was sixth most abundant miRNA detected (FIG. 9D). Consistent with our findings in mice, hsa-miR-6236 inhibited translation of human PTEN by directly binding to the 3′UTR of its mRNA (FIG. 10D). Furthermore, transfection of human primary adipocytes with hsa-MIR-6236 led to reduced PTEN protein levels and increased insulin-stimulated glucose uptake in vitro (FIGS. 8E, 8F). Together, these data indicate that the human genome harbors a hsa-miR-6236 homolog, the expression of which is modulated by obesity. Finally, we sought to determine whether hsa-miR-6236 is associated with any obesity-associated outcomes by reanalyzing adipose tissue miRNA sequencing data from ‘The Metabolic Syndrome in Men’ (METSIM) cohort (Laakso et al., 2017). METSIM is a large population-based study consisting of 10,197 Finnish male participants examined between 2005-2010 for numerous cardiometabolic traits such as BMI, oral glucose tolerance, insulin sensitivity, and various serum metabolites. We measured expression level hsa-miR-6236 in subcutaneous adipose tissue of 200 METSIM study participants by reanalyzing published small RNA-Seq data (Civelek et al., 2013). Then, we performed Pearson correlations between hsa-miR-6236 expression level and cardiometabolic traits. Notably, hsa-miR-6236 displays a significant negative correlation with fasting blood glucose level (p≤0.01) and OGTT 30′ blood glucose level (p≤0.05), and a positive correlation with insulin sensitivity (p<0.03) (FIGS. 9E, 9F). It is notable that hsa-miR-6236 expression did not significantly correlate with other cardiometabolic traits analyzed (FIG. 8C). Together, these data indicate that hsa-miR-6236 is associated with key obesity-associated outcomes and support conserved functions for murine and human miR-6236 in mammalian metabolism.

### DISCUSSION

The discovery of a molecule with the potential to counteract insulin resistance and obesity-associated hyperglycemia is of high relevance to the development of novel therapeutic agents for type 2 diabetes mellitus (T2DM). Here, we identified and defined the molecular action of a novel, myeloid cell-derived miRNA that influences adipocyte insulin signaling and global glucose homeostasis during obesity. The presence of a human homolog of miR-6236, with metabolic associations consistent with its mechanism in mice, suggest that this molecule has high translational potential.

In recent years, EV mediated delivery of miRNAs has emerged as an important mechanism by which ATMs can modulate adipose tissue insulin signaling (Liu et al., 2019; Ying et al., 2017, 2021). As ATMs are a heterogeneous cell group with both beneficial and harmful effects on mammalian metabolism depending on context (Hill et al., 2014; Jaitin et al., 2019; Russo and Lumeng, 2018), it is critical to investigate EV miRNA cargo from isolated ATM subsets with presumably homogenous functions. LAMs, which have predominantly beneficial functions in the context of obesity (Jaitin et al., 2019), serve as an ideal cell type to discover metabolically beneficial miRNAs. In this study, we identified miR-6236 as the most abundant miRNA secreted by LAMs and showed that miR-6236 counteracts development of obesity-associated insulin resistance by inhibiting the translation of adipocyte PTEN, a master negative regulator of insulin signaling pathway (FIG. 13) (Butler et al., 2002). Thus, miR-6236 mediated regulation of PTEN translation in adipocytes represents one mechanism by which LAMs exert their beneficial functions during obesity.

Our data indicate that miR-6236 can act in a macrophage-extrinsic manner to regulate adipocyte insulin-dependent functions. The fact that LAMs secrete more miR-6236-containing EVs than other adipose tissue myeloid cells, and are the predominant eWAT macrophage sub-type during obesity, may explain why we detected a trend towards reduced glucose accumulation in eWAT of DIO KO mice but no other adipose tissue depots. However, we also observed a trend towards reduced glucose uptake by the vastus lateralis muscle of DIO KO mice in our study. As such, there could be both paracrine and endocrine-like functions for miR-6236-containing EVs. In addition, we observed minimal macrophage-intrinsic effects of miR-6236 under the conditions we tested. However, we did observe significant changes in ATM and LAM gene expression in vivo when comparing DIO KO to DIO WT mice. As the current study is not able to determine whether these observed gene changes are the cause or an effect of the broader adipose tissue phenotype of miR-6236-deficient mice, it would be inappropriate to rule out macrophage-intrinsic functions for miR-6236. It is also notable that miR-6236 is expressed by non-ATM myeloid cells (e.g. neutrophils, monocytes, and Kupffer cells), and that it could be expressed by other non-myeloid cells. These other sources of miR-6236 may be physiologically-relevant both in obesity and in other disease settings. Conversely, miR-6236 seems to be expressed at a very low level by adipocytes, which is perhaps the reason why adipocytes deficient in miR-6236 show similar differentiation and insulin-dependent functional capacity as miR-6236-sufficient adipocytes.

Our data indicate that miR-6236 is an important regulator of adipose tissue insulin signaling and systemic glucose homeostasis during obesity. Herein, we characterize the effects of miR-6236 on PTEN in detail. However, we also show that miR-6236 can bind to and regulate the expression of Prkca mRNA. The regulation of multiple molecules that are central to insulin signaling may explain why miR-6236 KO mice have such a profound dysregulation of systemic glucose homeostasis. As an example, the DIO model in C57BL/6J mice often leads to weight gain and hyperinsulinemia, with limited hyperglycemia (Burke et al., 2017). In contrast, global or myeloid-specific deletion of miR-6236 results in marked obesity-associated hyperglycemia in the diabetic range (>250 mg/dl) without detectable difference in weight gain.

It is notable that female miR-6236 knockout mice develop obesity-associated hyperglycemia and hyperinsulinemia, despite resistance of female mice to DIO related metabolic complications (Pettersson et al., 2012). In addition, KO mice display greater adipose tissue lipolysis, and reduced AKT2 phosphorylation, despite having an elevated serum insulin level, indicating a significant disruption in insulin-dependent adipocyte functions. Finally, of all of 44 anthropogenic traits analyzed miR-6236 has the strongest correlation with fasting glycemia. While myeloid-specific deletion of miR-6236 recapitulated all the phenotypes of whole-body KO mice that we tested, the magnitude of the effect was slightly less for some measures. Together, these observations speak to high degree of relevance miR-6236 has to adipocyte insulin signaling and global glucose homeostasis during obesity.

MicroRNA sequences and their binding sites in 3′UTR of target genes are highly conserved across species (Chen and Rajewsky, 2006; Lagos-Quintana et al., 2001). However, ~50% of the mice miRNAs do not have known human homolog which limits their translational potential. As an example, current miRbase release 22.1 lists 1,917 miRNAs in human genome and 1,234 and miRNAs in the mouse genome, respectively. Of these, only 622 miRNAs are homologues between the two species (Kozomara et al., 2019). In addition, discovery of miRNA genes is challenging due to their smaller size and complex biogenesis (Gomes et al., 2013). To overcome these challenges, we performed extensive re-analyses of legacy transcriptomic and epigenomic sequencing data coupled with homolog sequence alignment and RNA secondary structure prediction for human miR-6236 discovery and annotation. The newly annotated miR-6236 meets all the characteristics of a miRNA gene i. e. existence of all 3 miRNA transcript forms in cell (e. g. pri-mRNA, pre-miRNA and mature sequence), miRNA-like stem-loop structure in pri/pre-miRNA transcripts, sequence conservation, and euchromatic nature of predicted gene locus. In addition to sequence and functional conservation between mice and humans, presence of abundant miR-6236 in serum EVs of obese patients, along with the high degree of association that MIR-6236 has with fasting glycemia, indicate that miR-6236 secretion by LAMs may be a conserved paracrine mechanism to provide metabolic adaptation during excessive caloric intake and obesity.

Though miRNA therapeutics are still in infancy with no FDA approved drugs, the field is growing rapidly as indicated by hundreds of recent patents awarded to the method or molecule that target miRNA for the treatment of various diseases including metabolic diseases (Chakraborty et al., 2021). More recently, circulating miRNAs have been proposed as disease biomarkers which relies on the fact that tissues enhance or suppress release of particular miRNA in extracellular body fluids via EVs depending on the context. Several studies are in different stages of clinical trials to develop circulating miRNAs as biomarkers to monitor the disease risk, diagnosis, disease progression and treatment response of obesity and obesity-associated diseases (Abbas, 2020; Guo, 2016; Holbaek Sygehus, 2019; LABAYEN, 2020; National Taiwan University Hospital, 2021; Solini, 2020). The ability of miR-6236 to robustly control obesity-associated insulin resistance and hyperglycemia make it a suitable molecule to develop as a therapeutic agent for the treatment of T2D. Similarly, obesity-regulated release of miR-6236 in serum, and its expression correlation with T2D outcomes, provide a new biomarker for diagnosis and monitoring of T2D progression and treatment.

### REFERENCES

Abbas, N.M. (2020). Hepatic Micro RNA Expression In Non Alcoholic Fatty Liver Disease (clinicaltrials.gov).
Abel, E.D., Peroni, O., Kim, J. K., Kim, Y.-B., Boss, O., Hadro, E., Minnemann, T., Shulman, G. I., and Kahn, B. B. (2001). Adipose-selective targeting of the GLUT4 gene impairs insulin action in muscle and liver. Nature 409, 729-733. https://doi.org/10.1038/35055575.
Akers, J.C., Gonda, D., Kim, R., Carter, B. S., and Chen, C.C. (2013). Biogenesis of extracellular vesicles (EV): exosomes, microvesicles, retrovirus-like vesicles, and apoptotic bodies. J Neurooncol 113, 1-11. https://doi.org/10.1007/s11060-013-1084-8.
Aparicio-Puerta, E., Lebrón, R., Rueda, A., Gómez-Martín, C., Giannoukakos, S., Jaspez, D., Medina, J.M., Zubkovic, A., Jurak, I., Fromm, B., et al. (2019). sRNAbench and sRNAtoolbox 2019: intuitive fast small RNA profiling and differential expression. Nucleic Acids Research 47, W530-W535. https://doi.org/10.1093/nar/gkz415.
Arner, P. (2002). Insulin resistance in type 2 diabetes: role of fatty acids. Diabetes/Metabolism Research and Reviews 18, S5-S9. https://doi.org/10.1002/dmrr.254.
Balakrishnan, I., Yang, X., Brown, J., Ramakrishnan, A., Torok-Storb, B., Kabos, P.,
Hesselberth, J. R., and Pillai, M.M. (2014). Genome-wide analysis of miRNA-mRNA interactions in marrow stromal cells. Stem Cells 32, 662-673. https://doi.org/10.1002/stem.1531. Blüher, M. (2013). Adipose tissue dysfunction contributes to obesity related metabolic diseases. Best Practice & Research Clinical Endocrinology & Metabolism 27, 163-177. https://doi.org/10.1016/j.beem.2013.02.005.
Burke, S. J., Batdorf, H.M., Burk, D.H., Noland, R.C., Eder, A.E., Boulos, M.S., Karlstad, M.D., and Collier, J.J. (2017). db/db Mice Exhibit Features of Human Type 2 Diabetes That Are Not Present in Weight-Matched C57BL/6J Mice Fed a Western Diet. J Diabetes Res 2017, 8503754. https://doi.org/10.1155/2017/8503754.
Butler, M., Mckay, R.A., Popoff, I.J., Gaarde, W.A., Witchell, D., Murray, S.F., Dean, N.M.,
Bhanot, S., and Monia, B.P. (2002). Specific Inhibition of PTEN Expression Reverses Hyperglycemia in Diabetic Mice. Diabetes 57, 1028-1034. https://doi.org/10.2337/diabetes.51.4.1028.
Chakraborty, C., Sharma, A.R., Sharma, G., and Lee, S.-S. (2021). Therapeutic advances of miRNAs: A preclinical and clinical update. Journal of Advanced Research 28, 127-138. https://doi.org/10.1016/j.jare.2020.08.012.
Chang, T.-C., Pertea, M., Lee, S., Salzberg, S. L., and Mendell, J.T. (2015). Genome-wide annotation of microRNA primary transcript structures reveals novel regulatory mechanisms. Genome Res 25, 1401-1409. https://doi.org/10.1101/gr.193607.115.
Chen, K., and Rajewsky, N. (2006). Deep conservation of microRNA-target relationships and 3′UTR motifs in vertebrates, flies, and nematodes. Cold Spring Harb Symp Quant Biol 149-156.
Civelek, M., Hagopian, R., Pan, C., Che, N., Yang, W., Kayne, P.S., Saleem, N.K., Cederberg, H., Kuusisto, J., Gargalovic, P. S., et al. (2013). Genetic regulation of human adipose microRNA expression and its consequences for metabolic traits. Hum Mol Genet 22, 3023-3037. https://doi.org/10.1093/hmg/ddt159.
Eulalio, A., Huntzinger, E., and Izaurralde, E. (2008). Getting to the Root of miRNA-Mediated Gene Silencing. Cell 132, 9-14. https://doi.org/10.1016/j.cell.2007.12.024.
Gomes, C., Cho, J.-H., Hood, L., Franco, O., Pereira, R., and Wang, K. (2013). A Review of Computational Tools in microRNA Discovery. Frontiers in Genetics 4 . . .
Guo, X. (2016). Circulating microRNAs as Novel Prognostic Biomarkers in Obese Preschoolers at Risk for Type 2 Diabetes in Adulthood (clinicaltrials.gov).
Haecker, I., Gay, L.A., Yang, Y., Hu, J., Morse, A.M., McIntyre, L. M., and Renne, R. (2012). Ago HITS-CLIP expands understanding of Kaposi's sarcoma-associated herpesvirus miRNA function in primary effusion lymphomas. PLOS Pathog 8, e1002884. https://doi.org/10.1371/journal.ppat.1002884.
Hill, A.A., Reid Bolus, W., and Hasty, A.H. (2014). A decade of progress in adipose tissue macrophage biology. Immunological Reviews 262, 134-152. https://doi.org/10.1111/imr.12216.
Hill, D.A., Lim, H.-W., Kim, Y.H., Ho, W.Y., Foong, Y.H., Nelson, V.L., Nguyen, H.C.B., Chegireddy, K., Kim, J., Habertheuer, A., et al. (2018). Distinct macrophage populations direct inflammatory versus physiological changes in adipose tissue. Proc Natl Acad Sci USA 115, E5096-E5105. https://doi.org/10.1073/pnas.1802611115.
Holbaek Sygehus (2019). MicroRNA as Biomarkers for Development of Metabolic Syndrome in Women With Polycystic Ovary Syndrome (clinicaltrials.gov).
Jaitin, D.A., Adlung, L., Thaiss, C.A., Weiner, A., Li, B., Descamps, H., Lundgren, P., Bleriot, C., Liu, Z., Deczkowska, A., et al. (2019). Lipid-Associated Macrophages Control Metabolic Homeostasis in a Trem2-Dependent Manner. Cell 178, 686-698.e14. https://doi.org/10.1016/j.cell.2019.05.054.
Kahn, S.E., Hull, R. L., and Utzschneider, K.M. (2006). Mechanisms linking obesity to insulin resistance and type 2 diabetes. Nature 444, 840-846. https://doi.org/10.1038/nature05482.
Kern, F., Amand, J., Senatorov, I., Isakova, A., Backes, C., Meese, E., Keller, A., and Fehlmann,
T. (2020). miRSwitch: detecting microRNA arm shift and switch events. Nucleic Acids Research 48, W268-W274. https://doi.org/10.1093/nar/gkaa323.
Kim, D., Paggi, J.M., Park, C., Bennett, C., and Salzberg, S.L. (2019). Graph-based genome alignment and genotyping with HISAT2 and HISAT-genotype. Nat Biotechnol 37, 907-915. https://doi.org/10.1038/s41587-019-0201-4.
Kozomara, A., Birgaoanu, M., and Griffiths-Jones, S. (2019). miRBase: from microRNA sequences to function. Nucleic Acids Research 47, D155-D162. https://doi.org/10.1093/nar/gkyl141.
Kusminski, C.M., Bickel, P. E., and Scherer, P.E. (2016). Targeting adipose tissue in the treatment of obesity-associated diabetes. Nat Rev Drug Discov 15, 639-660. https://doi.org/10.1038/nrd.2016.75.
Laakso, M., Kuusisto, J., Stančáková, A., Kuulasmaa, T., Pajukanta, P., Lusis, A.J., Collins, F.S., Mohlke, K. L., and Boehnke, M. (2017). The Metabolic Syndrome in Men study: a resource for studies of metabolic and cardiovascular diseases. J Lipid Res 58, 481-493. https://doi.org/10.1194/jlr.0072629.
LABAYEN, I. (2020). Prevention of Diabetes in Overweight/Obese Preadolescent Children Through a Family-based Intervention Program Including Supervised Exercise; the PREDIKID Study (clinicaltrials.gov).
Lagos-Quintana, M., Rauhut, R., Lendeckel, W., and Tuschl, T. (2001). Identification of Novel Genes Coding for Small Expressed RNAs. Science 294, 853-858. https://doi.org/10.1126/science.1064921.
Li, Y. Z., Di Cristofano, A., and Woo, M. (2020). Metabolic Role of PTEN in Insulin Signaling and Resistance. Cold Spring Harb Perspect Med 10, a036137. https://doi.org/10.1101/cshperspect.a036137.
Liu, T., Sun, Y.-C., Cheng, P., and Shao, H.-G. (2019). Adipose tissue macrophage-derived exosomal miR-29a regulates obesity-associated insulin resistance. Biochemical and Biophysical Research Communications 515, 352-358. https://doi.org/10.1016/j.bbrc.2019.05.113.
Maehama, T., and Dixon, J. E. (1998). The Tumor Suppressor, PTEN/MMAC1, Dephosphorylates the Lipid Second Messenger, Phosphatidylinositol 3,4,5-Trisphosphate *. Journal of Biological Chemistry 273, 13375-13378. https://doi.org/10.1074/jbc.273.22.13375.
Mathis, D. (2013). Immunological goings-on in visceral adipose tissue. Cell Metab 17, 851-859. https://doi.org/10.1016/j.cmet.2013.05.008.
Mukaida, S., Evans, B. A., Bengtsson, T., Hutchinson, D. S., and Sato, M. (2017). Adrenoceptors promote glucose uptake into adipocytes and muscle by an insulin-independent signaling pathway involving mechanistic target of rapamycin complex 2. Pharmacological Research 116, 87-92. https://doi.org/10.1016/j.phrs.2016.12.022.
Nakashima, N., Sharma, P. M., Imamura, T., Bookstein, R., and Olefsky, J. M. (2000). The Tumor Suppressor PTEN Negatively Regulates Insulin Signaling in 3T3-L1 Adipocytes *. Journal of Biological Chemistry 275, 12889-12895. https://doi.org/10.1074/jbc.275.17.12889. National Taiwan University Hospital (2021). Circulating microRNAs and Adverse Cardiovascular Outcomes in Patients With Coronary Artery Disease (clinicaltrials.gov).
Nelson, M. E., Madsen, S., Cooke, K. C., Fritzen, A. M., Thorius, I. H., Masson, S. W. C., Carroll, L., Weiss, F. C., Seldin, M. M., Potter, M., et al. (2022). Systems-level analysis of insulin action in mouse strains provides insight into tissue- and pathway-specific interactions that drive insulin resistance. Cell Metabolism 34, 227-239.e6. https://doi.org/10.1016/j.cmet.2021.12.013.
O'Brien, J., Hayder, H., Zayed, Y., and Peng, C. (2018). Overview of MicroRNA Biogenesis, Mechanisms of Actions, and Circulation. Frontiers in Endocrinology 9 . . . .
O'Connor, S., Murphy, E. A., Szwed, S. K., Kanke, M., Marchildon, F., Sethupathy, P., Darnell, R. B., and Cohen, P. (2021). AGO HITS-CLIP reveals distinct miRNA regulation of white and brown adipose tissue identity. Genes Dev. 35, 771-781. https://doi.org/10.1101/gad.345447.120.
Perrin, H. J., Currin, K. W., Vadlamudi, S., Pandey, G. K., Ng, K. K., Wabitsch, M., Laakso, M., Love, M. I., and Mohlke, K. L. (2021). Chromatin accessibility and gene expression during adipocyte differentiation identify context-dependent effects at cardiometabolic GWAS loci. PLOS Genet 17, e1009865. https://doi.org/10.1371/journal.pgen.1009865.
Pertea, M., Pertea, G.M., Antonescu, C.M., Chang, T.-C., Mendell, J. T., and Salzberg, S.L. (2015). StringTie enables improved reconstruction of a transcriptome from RNA-seq reads. Nat Biotechnol 33, 290-295. https://doi.org/10.1038/nbt.3122.
Pettersson, U.S., Walden, T.B., Carlsson, P.-O., Jansson, L., and Phillipson, M. (2012). Female Mice are Protected against High-Fat Diet Induced Metabolic Syndrome and Increase the Regulatory T Cell Population in Adipose Tissue. PLOS ONE 7, e46057. https://doi.org/10.1371/journal.pone.0046057.
Reaven, G.M. (1988). Role of Insulin Resistance in Human Disease. Diabetes 37, 1595-1607. https://doi.org/10.2337/diab.37.12.1595.
Russo, L., and Lumeng, C. N. (2018). Properties and functions of adipose tissue macrophages in obesity. Immunology 155, 407-417. https://doi.org/10.1111/imm.13002.
Santoro, A., McGraw, T. E., and Kahn, B.B. (2021). Insulin action in adipocytes, adipose remodeling, and systemic effects. Cell Metabolism 33, 748-757. https://doi.org/10.1016/j.cmet.2021.03.019.
Solini, A. (2020). Association Between Urinary and Serum Levels of miRNA 192 and miRNA 25 and Glomerular Filtration and Albuminuria in Patients With and Without Type 2 Diabetes. (clinicaltrials.gov).
Sostre-Colón, J., Uehara, K., Garcia Whitlock, A.E., Gavin, M.J., Ishibashi, J., Potthoff, M. J., Seale, P., and Titchenell, P. M. (2021). Hepatic AKT orchestrates adipose tissue thermogenesis via FGF21-dependent and-independent mechanisms. Cell Rep 35, 109128. https://doi.org/10.1016/j.celrep.2021.109128.
Sung, H.-K., Doh, K.-O., Son, J. E., Park, J. G., Bae, Y., Choi, S., Nelson, S. M. L., Cowling, R., Nagy, K., Michael, I. P., et al. (2013). Adipose vascular endothelial growth factor regulates metabolic homeostasis through angiogenesis. Cell Metab 17, 61-72. https://doi.org/10.1016/j.cmet.2012.12.010.
Weyer, C., Foley, J. E., Bogardus, C., Tataranni, P. A., and Pratley, R. E. (2000). Enlarged subcutaneous abdominal adipocyte size, but not obesity itself, predicts Type II diabetes independent of insulin resistance. Diabetologia 43, 1498-1506. https://doi.org/10.1007/s001250051560.
Ying, W., Riopel, M., Bandyopadhyay, G., Dong, Y., Birmingham, A., Seo, J.B., Ofrecio, J.M., Wollam, J., Hernandez-Carretero, A., Fu, W., et al. (2017). Adipose Tissue Macrophage-Derived Exosomal miRNAs Can Modulate In Vivo and In Vitro Insulin Sensitivity. Cell 171, 372-384.e12. https://doi.org/10.1016/j.cell.2017.08.035.
Ying, W., Gao, H., Reis, F. C. G. D., Bandyopadhyay, G., Ofrecio, J. M., Luo, Z., Ji, Y., Jin, Z., Ly, C., and Olefsky, J. M. (2021). MiR-690, an exosomal-derived miRNA from M2-polarized macrophages, improves insulin sensitivity in obese mice. Cell Metabolism 33, 781-790.e5. https://doi.org/10.1016/j.cmet.2020.12.019.

While certain of the preferred embodiments of the present invention have been described and specifically exemplified above, it is not intended that the invention be limited to such embodiments. It will be apparent to one skilled in the art that various changes and modifications can be made therein without departing from the scope of the present invention, as set forth in the following claims.

1. A composition comprising a hsa-miR6236 or a synthetic, functional variant thereof in a pharmaceutically acceptable carrier for treatment one or more obesity related metabolic disorders in a human subject in need of treatment thereof.

2. The composition of claim 1, wherein the has-miR6236 or synthetic, functional variant thereof encodes at least one of SEQ ID NOs: 58-85, one of the sequences listed in Table 2, or a sequence having at least 95% identity to said sequences.

3. (canceled)

4. The composition of claim 1, wherein the has-miR6236 or synthetic, functional variant thereof encodes at least one of sequence selected from:
i) SEQ ID NOs: 58-62, or
ii) SEQ ID NOs: 63-68, or
iii) SEQ ID NOs: 69-73, or
iv) SEQ ID NOs: 74-78, or
v) SEQ ID NOs: 79-82, or
vi) SEQ ID NOs: 82-85.

5. The composition of claim 1, wherein said disorder is type 2 diabetes or cardiovascular disease.

6. (canceled)

7. The composition of claim 1, wherein said subject has a genetic or epigenetic predisposition to obesity.

8. The composition of claim 1, for modulation of PTEN activity in a tissue selected from brown fat, white fat, subcutaneous adipose tissue, liver and muscle.

9. The composition of claim 1, wherein said synthetic hsa-miR6236 comprises chemical modifications for enhancing stability and, or, bioavailability in the body, and alleviates symptoms of said one or more obesity related metabolic disorders.

10. The composition of claim 1, for reducing body weight in said subject, wherein said miR6236 or a synthetic, functional variant thereof is operably linked to a nanoparticle or present in an exosome or extracellular vesicle, or a targeting molecule for specific delivery to a specific cell type or tissue.

11. (canceled)

12. The composition of claim 1, said miRNA6236 or functional variant thereof binding PTEN mRNA and inhibiting PTEN protein expression and function.

13. An exosome pellet, extracellular vessicle, or physiological solution isolated from a biological sample comprising exosomes harboring miRNA6236 or a functional synthetic variant thereof.

14. The pellet or solution of claim 13, wherein the has-miR6236 or synthetic, functional variant thereof encodes at least one of SEQ ID NOs: 58-85, one of the sequences listed in Table 2, or a sequence having at least 95% identity to said sequences.

15.-16. (canceled)

17. The pellet or solution of claim 13, which is essentially free from undesirable entities or cellular debris having a diameter less than 20 nm and greater than 140 nm.

18. The pellet or solution of claim 13, wherein the exosomes are isolated from CD9+LAM cells.

19.-20. (canceled)

21. A method of treating obesity related metabolic dysfunction in a subject in need thereof, the method comprising administering an effective amount of the composition of claim 1.

22. (canceled)

23. A method of treating obesity related metabolic dysfunction in a subject in need thereof, the method comprising administering an effective amount of the exosome pellet or physiological solution comprising the exosomes of claim 13.

24. The method of claim 23, wherein the exosomes are essentially free from undesirable entities having a diameter less than 20 nm and greater than 140 nm.

25.-27. (canceled)

28. The method of claim 21, to treat metabolic syndrome, cardiovascular disease, and hypertension.

29. The method of claim 28, wherein the metabolic syndrome is selected from the group consisting of obesity, metabolic syndrome, type 2 diabetes, non-alcoholic fatty liver disease, hyperinsulinemia, hypoinsulinemia, hypertension, hyperhepatosteatosis, hyperuricemia, fatty liver, polycystic ovarian syndrome, hyperphagia, acanthosis nigricans, endocrine abnormalities, triglyceride storage disease, Bardet-Biedl syndrome, Lawrence-Moon syndrome, Parder-Labhart-Willi syndrome, a primary mitochondrial genetic disorder, a neurological disease, and an age-associated pathology.

30. A diagnostic or prognostic method for identifying a subject at altered risk for one or more obesity related metabolic disorders, comprising;
a) detecting levels of free hsa-miR6236 in circulation or hsa-miR6236 within an extracellular vesicle or endosome in a biological sample obtained from a subject; and
b) comparing the levels detected in step a) with those from control subjects without disease and from subjects with at least one obesity related metabolic disorder, thereby identifying a subject as being at greater or lesser risk for an obesity disorder.

31.-34. (canceled)

35. The method of claim 30, wherein
a) reduced levels of miR6236 levels are detected in the subject relative to a control subject without disease thereby identifying a subject with an increased risk of said metabolic disorder, or
b) elevated levels of miR6236 levels are detected in the subject relative to a control subject without disease, thereby identifying a subject with a decreased risk of said metabolic disorder.

36. (canceled)


SEQUENCE LISTING (85 sequences)

SEQ ID NO: 1 - RNA, 123 nt, synthetic construct
  source 1..123: mol_type = other RNA
acaacccacctgccgaatcaactagccctgaaaatggatggcgctggagcgtggggaccatacccagccgtcgccggcagtcaggggtgggggtgggggtggagaaagaaaaaaaatcaataa

SEQ ID NO: 2 - DNA, 447 bp, synthetic construct
  source 1..447: mol_type = other DNA
  misc_feature 5..27: note = Targeting Primer
  misc_feature 421..443: note = Targeting Primer
  misc_feature 192..314: note = pre-miR-6236
aactgctgatttccagcaacacagaggagagtgactccaaaaaataactttctaaacatgcccacttcccccataccctttcttttccactacctctggtagttggtagactacaagagaggttaaaatgtttaaaaatcattaaaaatggacagtggccatggaagtagaaatctgctaaggagtgtgtaacaactcacctgccgaatcaactagccctgaaaatggatggcgctggagcgtggggaccatacccagccgtcgccggcagtcaggggtgggggtgggggtggagaaagaaaaaaaatcaataaaaataaagtctaaaaagttaaagttactgttgggtgtcttcccagctgggctggtaaaccatgaaaggtctccctccctcagggataggtggggcaatggtgtgcagagggaaagctgcggttgaagggctga

SEQ ID NO: 3 - DNA, 349 bp, synthetic construct
  source 1..349: mol_type = other DNA
  misc_feature 6..12: note = binding site
  misc_feature 337..344: note = binding site
gaagtttttcagtagaaattgtcctacagatgctttatcgacttgctattggaagaatagatcttcttaaatgtgcagtgttgagtcacttcgttatagtggtagagttgggattagggcttcaattttacttcttaaatatcattctatgtttgatatgcccagactgcatacaatttaaagcaagagtacaactactatcgtaatggtaatgtgaagatgctattacaaaggatctcctcccaacccctcgggaatttggtgtctttcaaattatatcttgaccttgacatttgaatatccagccattattagatttcttaatggtgtgaagtcccattttcaataa

SEQ ID NO: 4 - RNA, 24 nt, synthetic construct
  source 1..24: mol_type = other RNA
aataaggcattgaagtttttcagt

SEQ ID NO: 5 - RNA, 22 nt, synthetic construct
  source 1..22: mol_type = other RNA
atggtgtgaagtcccattttca

SEQ ID NO: 6 - RNA, 447 nt, synthetic construct
  source 1..447: mol_type = other RNA
aactgctgatttccagcaacacagaggagagtgactccaaaaaataactttctaaacatgcccacttcccccataccctttcttttccactacctctggtagttggtagactacaagagaggttaaaatgtttaaaaatcattaaaaatggacagtggccatggaagtagaaatctgctaaggagtgtgtaacaactcacctgccgaatcaactagccctgaaaatggatggcgctggagcgtggggaccatacccagccgtcgccggcagtcaggggtgggggtgggggtggagaaagaaaaaaaatcaataaaaataaagtctaaaaagttaaagttactgttgggtgtcttcccagctgggctggtaaaccatgaaaggtctccctccctcagggataggtggggcaatggtgtgcagagggaaagctgcggttgaagggctga

SEQ ID NO: 7 - RNA, 90 nt, synthetic construct
  source 1..90: mol_type = other RNA
acaactcacctgccgaatcaactagccctgaaaatggatggcgctggagcgttgggaggctaccaggctgttgccggcagtcgagagtgg

SEQ ID NO: 8 - RNA, 123 nt, synthetic construct
  source 1..123: mol_type = other RNA
acaactcacctgccgaatcaactagccctgaaaatggatggcgctggagcgtngggannntaccnngcngtngccggcagtcnngngtgggggtgggggtggagaaagaaaaaaaatcaataa

SEQ ID NO: 9 - RNA, 123 nt, synthetic construct
  source 1..123: mol_type = other RNA
acaactcacctgccgaatcaactagccctgaaaatggatggcgctggagcgtkgggassmtaccmrgcygtygccggcagtcrrgrgtgggggtgggggtggagaaagaaaaaaaatcaataa

SEQ ID NO: 10 - RNA, 36 nt, synthetic construct
  source 1..36: mol_type = other RNA
agtcatgctagctctcagttgtaatgactgctccat

SEQ ID NO: 11 - RNA, 40 nt, synthetic construct
  source 1..40: mol_type = other RNA
tatgttcctgcagggtgtaaatcctaagagctttgttaca

SEQ ID NO: 12 - RNA, 18 nt, synthetic construct
  source 1..18: mol_type = other RNA
cgcagccactgtcgagtc

SEQ ID NO: 13 - RNA, 20 nt, synthetic construct
  source 1..20: mol_type = other RNA
gtcatccatggcgaactggt

SEQ ID NO: 14 - RNA, 19 nt, synthetic construct
  source 1..19: mol_type = other RNA
ttcagtattggacgcgagg

SEQ ID NO: 15 - RNA, 20 nt, synthetic construct
  source 1..20: mol_type = other RNA
gaatctgacacccagcggaa

SEQ ID NO: 16 - RNA, 20 nt, synthetic construct
  source 1..20: mol_type = other RNA
gtacattggcttgcgagacg

SEQ ID NO: 17 - RNA, 21 nt, synthetic construct
  source 1..21: mol_type = other RNA
cggccttttcttccttcccag

SEQ ID NO: 18 - RNA, 20 nt, synthetic construct
  source 1..20: mol_type = other RNA
gatcgcttgcccaactcttg

SEQ ID NO: 19 - RNA, 20 nt, synthetic construct
  source 1..20: mol_type = other RNA
actgtgacatccttgggctg

SEQ ID NO: 20 - RNA, 20 nt, synthetic construct
  source 1..20: mol_type = other RNA
ggtgcctatgtctcagcctc

SEQ ID NO: 21 - RNA, 20 nt, synthetic construct
  source 1..20: mol_type = other RNA
gctcctccacttggtggttt

SEQ ID NO: 22 - RNA, 20 nt, synthetic construct
  source 1..20: mol_type = other RNA
tcttcgggttcctcttggtg

SEQ ID NO: 23 - RNA, 20 nt, synthetic construct
  source 1..20: mol_type = other RNA
gggttcatccttgctccgta

SEQ ID NO: 24 - RNA, 20 nt, synthetic construct
  source 1..20: mol_type = other RNA
gcaagtggtccgattccctt

SEQ ID NO: 25 - RNA, 20 nt, synthetic construct
  source 1..20: mol_type = other RNA
ggagcaaagcaggctcagta

SEQ ID NO: 26 - RNA, 20 nt, synthetic construct
  source 1..20: mol_type = other RNA
tcgtcatcgagaggatccga

SEQ ID NO: 27 - RNA, 20 nt, synthetic construct
  source 1..20: mol_type = other RNA
tgtttgtccagtgtcagcca

SEQ ID NO: 28 - RNA, 20 nt, synthetic construct
  source 1..20: mol_type = other RNA
tccctcagctctcctgttag

SEQ ID NO: 29 - RNA, 20 nt, synthetic construct
  source 1..20: mol_type = other RNA
tgacataagcggaggacaca

SEQ ID NO: 30 - RNA, 20 nt, synthetic construct
  source 1..20: mol_type = other RNA
agaaggatcgaagaaccgca

SEQ ID NO: 31 - RNA, 21 nt, synthetic construct
  source 1..21: mol_type = other RNA
gtgtgagaacgctgaggcttt

SEQ ID NO: 32 - RNA, 20 nt, synthetic construct
  source 1..20: mol_type = other RNA
cacctgccgaatcaactagc

SEQ ID NO: 33 - RNA, 19 nt, synthetic construct
  source 1..19: mol_type = other RNA
acctgccgaatcaactagc

SEQ ID NO: 34 - RNA, 21 nt, synthetic construct
  source 1..21: mol_type = other RNA
tcacctgccgaatcaactagc

SEQ ID NO: 35 - RNA, 23 nt, synthetic construct
  source 1..23: mol_type = other RNA
actcacctgccgaatcaactagc

SEQ ID NO: 36 - RNA, 24 nt, synthetic construct
  source 1..24: mol_type = other RNA
cctgaaaatggatggcgctggagc

SEQ ID NO: 37 - RNA, 19 nt, synthetic construct
  source 1..19: mol_type = other RNA
ccgaatcaactagccctga

SEQ ID NO: 38 - RNA, 20 nt, synthetic construct
  source 1..20: mol_type = other RNA
cgaatcaactagccctgaaa

SEQ ID NO: 39 - RNA, 22 nt, synthetic construct
  source 1..22: mol_type = other RNA
ctcacctgccgaatcaactagc

SEQ ID NO: 40 - RNA, 24 nt, synthetic construct
  source 1..24: mol_type = other RNA
aactcacctgccgaatcaactagc

SEQ ID NO: 41 - RNA, 25 nt, synthetic construct
  source 1..25: mol_type = other RNA
caactcacctgccgaatcaactagc

SEQ ID NO: 42 - RNA, 21 nt, synthetic construct
  source 1..21: mol_type = other RNA
ccctgaaaatggatggcgctg

SEQ ID NO: 43 - RNA, 23 nt, synthetic construct
  source 1..23: mol_type = other RNA
ctgaaaatggatggcgctggagc

SEQ ID NO: 44 - RNA, 23 nt, synthetic construct
  source 1..23: mol_type = other RNA
ctgaaaatggatggcgctggagc

SEQ ID NO: 45 - RNA, 23 nt, synthetic construct
  source 1..23: mol_type = other RNA
tgccgaatcaactagccctgaaa

SEQ ID NO: 46 - RNA, 19 nt, synthetic construct
  source 1..19: mol_type = other RNA
gaatcaactagccctgaaa

SEQ ID NO: 47 - RNA, 22 nt, synthetic construct
  source 1..22: mol_type = other RNA
tgaaaatggatggcgctggagc

SEQ ID NO: 48 - RNA, 20 nt, synthetic construct
  source 1..20: mol_type = other RNA
aaaatggatggcgctggagc

SEQ ID NO: 49 - RNA, 20 nt, synthetic construct
  source 1..20: mol_type = other RNA
ctgccgaatcaactagccct

SEQ ID NO: 50 - RNA, 19 nt, synthetic construct
  source 1..19: mol_type = other RNA
aaatggatggcgctggagc

SEQ ID NO: 51 - RNA, 20 nt, synthetic construct
  source 1..20: mol_type = other RNA
gccgaatcaactagccctga

SEQ ID NO: 52 - RNA, 22 nt, synthetic construct
  source 1..22: mol_type = other RNA
acaactcacctgccgaatcaac

SEQ ID NO: 53 - RNA, 21 nt, synthetic construct
  source 1..21: mol_type = other RNA
cctgccgaatcaactagccct

SEQ ID NO: 54 - RNA, 27 nt, synthetic construct
  source 1..27: mol_type = other RNA
agccctgaaaatggatggcgctggagc

SEQ ID NO: 55 - RNA, 28 nt, synthetic construct
  source 1..28: mol_type = other RNA
tagccctgaaaatggatggcgctggagc

SEQ ID NO: 56 - RNA, 19 nt, synthetic construct
  source 1..19: mol_type = other RNA
aatcaactagccctgaaaa

SEQ ID NO: 57 - RNA, 26 nt, synthetic construct
  source 1..26: mol_type = other RNA
gccctgaaaatggatggcgctggagc

SEQ ID NO: 58 - RNA, 20 nt, synthetic construct
  source 1..20: mol_type = other RNA
aaaatggatggcgctggagc

SEQ ID NO: 59 - RNA, 20 nt, synthetic construct
  source 1..20: mol_type = other RNA
aaatggatggcgctggagcg

SEQ ID NO: 60 - RNA, 21 nt, synthetic construct
  source 1..21: mol_type = other RNA
aactagccctgaaaatggatg

SEQ ID NO: 61 - RNA, 22 nt, synthetic construct
  source 1..22: mol_type = other RNA
aaggagtgtgtaacaactcacc

SEQ ID NO: 62 - RNA, 22 nt, synthetic construct
  source 1..22: mol_type = other RNA
aatcaactagccctgaaaatgg

SEQ ID NO: 63 - RNA, 19 nt, synthetic construct
  source 1..19: mol_type = other RNA
aatggatggcgctggagcg

SEQ ID NO: 64 - RNA, 20 nt, synthetic construct
  source 1..20: mol_type = other RNA
actagccctgaaaatggatg

SEQ ID NO: 65 - RNA, 21 nt, synthetic construct
  source 1..21: mol_type = other RNA
aggagtgtgtaacaactcacc

SEQ ID NO: 66 - RNA, 21 nt, synthetic construct
  source 1..21: mol_type = other RNA
atcaactagccctgaaaatgg

SEQ ID NO: 67 - RNA, 22 nt, synthetic construct
  source 1..22: mol_type = other RNA
cacctgccgaatcaactagccc

SEQ ID NO: 68 - RNA, 21 nt, synthetic construct
  source 1..21: mol_type = other RNA
ccctgaaaatggatggcgctg

SEQ ID NO: 69 - RNA, 21 nt, synthetic construct
  source 1..21: mol_type = other RNA
ccggcagtcgagagtggacgg

SEQ ID NO: 70 - RNA, 20 nt, synthetic construct
  source 1..20: mol_type = other RNA
cctgaaaatggatggcgctg

SEQ ID NO: 71 - RNA, 22 nt, synthetic construct
  source 1..22: mol_type = other RNA
cctgccgaatcaactagccctg

SEQ ID NO: 72 - RNA, 24 nt, synthetic construct
  source 1..24: mol_type = other RNA
cgaatcaactagccctgaaaatgg

SEQ ID NO: 73 - RNA, 23 nt, synthetic construct
  source 1..23: mol_type = other RNA
cgctaaggagtgtgtaacaactc

SEQ ID NO: 74 - RNA, 20 nt, synthetic construct
  source 1..20: mol_type = other RNA
cggcagtcgagagtggacgg

SEQ ID NO: 75 - RNA, 24 nt, synthetic construct
  source 1..24: mol_type = other RNA
ctaaggagtgtgtaacaactcacc

SEQ ID NO: 76 - RNA, 19 nt, synthetic construct
  source 1..19: mol_type = other RNA
ctagccctgaaaatggatg

SEQ ID NO: 77 - RNA, 25 nt, synthetic construct
  source 1..25: mol_type = other RNA
ctcacctgccgaatcaactagccct

SEQ ID NO: 78 - RNA, 20 nt, synthetic construct
  source 1..20: mol_type = other RNA
ctgaaaatggatggcgctgg

SEQ ID NO: 79 - RNA, 21 nt, synthetic construct
  source 1..21: mol_type = other RNA
gaaaatggatggcgctggagc

SEQ ID NO: 80 - RNA, 23 nt, synthetic construct
  source 1..23: mol_type = other RNA
gaatcaactagccctgaaaatgg

SEQ ID NO: 81 - RNA, 22 nt, synthetic construct
  source 1..22: mol_type = other RNA
gccggcagtcgagagtggacgg

SEQ ID NO: 82 - RNA, 22 nt, synthetic construct
  source 1..22: mol_type = other RNA
gctaaggagtgtgtaacaactc

SEQ ID NO: 83 - RNA, 19 nt, synthetic construct
  source 1..19: mol_type = other RNA
ggcagtcgagagtggacgg

SEQ ID NO: 84 - RNA, 24 nt, synthetic construct
  source 1..24: mol_type = other RNA
tcacctgccgaatcaactagccct

SEQ ID NO: 85 - RNA, 22 nt, synthetic construct
  source 1..22: mol_type = other RNA
tgaaaatggatggcgctggagc